(19) United States
(12) Patent Application Publication
(10) Pub. No.: US 2020/0378956 A1
(43) Pub. Date: Dec. 3, 2020

(54) HUMAN MICROPHYSIOLOGICAL CELL SYSTEM FOR LIVER DISEASE CONVERSTION PROV 1-18585 AND PROV 2-19154

(71) Applicant: EMULATE, INC., Boston, MA (US)

(72) Inventors: Catherine Karalis, Brookline, MA (US); Deborah Barrillos Petropolis, Cambridge, MA (US)

(21) Appl. No.: 16/995,405

(22) Filed: Aug. 17, 2020

Related U.S. Application Data
(63) Continuation of application No. PCT/US2019/018787, filed on Feb. 20, 2019.
(60) Provisional application No. 62/632,893, filed on Feb. 20, 2018.
(60) Provisional application No. 62/758,158, filed on Nov. 9, 2018.

(51) Int. Cl. G01N 33/50; C12M 3/06; C12M 1/12; G01N 33/92

(52) U.S. Cl. CPC G01N 33/5091; C12M 23/16; C12M 25/14; G01N 2800/7009; G01N 2800/085; G01N 2800/709; G01N 2500/10; G01N 33/92

(57) ABSTRACT

The present invention is related to the field of liver disease. Solid substrates comprising microfluidic channels (e.g., microchips) are configured to support growing and differentiating hepatocytes and are contemplated to provide a suitable environment for the development of fully functional liver tissue. These solid substrates can be used to induce various toxicity conditions in the liver tissue subsequent to the exposure to various chemicals. For example, chronic exposure to ethanol induces a clinical state of alcoholic liver disease in the liver tissue. Alternatively, certain disease states can result in the development of non-alcoholic liver diseases (e.g., non- alcoholic steatohepatitis; NASH).

### FIELD OF THE INVENTION

The present invention is related to the field of liver disease. Solid substrates comprising microfluidic channels (e.g., microchips) are configured to support growing and differentiating hepatocytes and are contemplated to provide a suitable environment for the development of a fully functional liver tissue. These solid substrates can be used to induce various toxicity conditions in the liver tissue subsequent to the exposure to various chemicals. For example, chronic exposure to ethanol induces a clinical state of alcoholic liver disease in the liver tissue. Alternatively, certain disease states can result in the development of non-alcoholic liver diseases (e.g., non-alcoholic steatohepatitis; NASH).

### BACKGROUND

Liver diseases are believed to be one of the major causes of morbidity and mortality in the world. Some of the most common liver diseases, alcoholic liver disease (ALD) or non-alcoholic steatohepatitis (NASH), generally refer to a broad range of stages encountered during this progressive disease including, but not limited to, fatty liver, alcoholic steatohepatitis (ASH), liver fibrosis and/or liver cirrhosis that may progress into the development of hepatocellular carcinoma. Magdaleno et al., “Key Events Participating in the Pathogenesis of Alcoholic Liver Disease” Biomolecules 2017; 7: 9. Approximately 80-100% of individuals with excessive alcohol consumption develop ASH, of which approximately 10-40% progress into liver fibrosis and approximately 10% to 15% into liver cirrhosis. ASH can be reversed after 4-6 weeks of abstinence, however, liver cirrhosis is responsible for 70% to 80% of the directly recorded mortality from alcohol and is responsible for 47.9% of all liver cirrhosis deaths, representing 10.9% of all deaths regardless of the cause. Rehm et al., “Global burden of disease and injury and economic cost attributable to alcohol use and alcohol-use disorders” Lancet 2009; 373:2223-2233; and Stickel et al., “Pathophysiology and Management of Alcoholic Liver Disease: Update 2016” Gut and Liver 2017; 11:173-188.

What is needed in the art is a functional microphysiological liver tissue system for the identification of druggable biochemical mechanisms and to provide a screening platform for testing potential clinically efficacious lead compounds.

### SUMMARY OF THE INVENTION

The present invention is related to the field of liver disease. Solid substrates comprising microfluidic channels (e.g., microchips) are configured to support growing and differentiating hepatocytes and are contemplated to provide a suitable environment for the development of a fully functional liver tissue. These solid substrates can be used to induce various toxicity conditions in the liver tissue subsequent to the exposure to various chemicals. For example, chronic exposure to ethanol induces a clinical state of alcoholic liver disease in the liver tissue. Alternatively, certain disease states can result in the development of non-alcoholic liver diseases (e.g., non-alcoholic steatohepatitis; NASH).

In one embodiment, the present invention contemplates a microfluidic device comprising: a) a solid substrate comprising a membrane and one or more microfluidic channels; and b) hepatic cells, wherein said hepatic cells exhibit at least one liver disease biomarker (or indicator of a disease phenotype, such as lipid accumulation). In one embodiment, the present invention contemplates a microfluidic tissue testing device, comprising: a) a solid substrate comprising a single microfluidic channel; b) a porous membrane separating said single microfluidic channel into a first chamber and a second chamber; and c) a hepatic tissue comprising human cellular architecture attached to said porous membrane, wherein said hepatic tissue comprises at least one liver disease biomarker. In one embodiment, said hepatic tissue comprises a hepatocyte layer within said first chamber. In one embodiment, said hepatic tissue comprises an endothelial cell layer within said second chamber. In one embodiment, said endothelial cell layer further comprises Kupffer cells. In one embodiment, said endothelial cell layer further comprises stellate cells. In one embodiment, said microfluidic channel further comprises a blood vessel cell layer attached to said endothelial cell layer. In one embodiment, said solid substrate further comprises at least one inlet channel in fluid communication with said single microfluidic channel. In one embodiment, said solid substrate further comprises at least one outlet channel in fluidic communication with said single microfluidic channel. In one embodiment, the hepatic tissue further exhibits one or more symptoms or indicators of steatohepatitis. In one embodiment, said at least one liver disease biomarker is an alcoholic liver disease biomarker. In one embodiment, said at least one alcoholic liver disease biomarker is selected from the group consisting of lipid droplets, cytochrome P450 induction, hepatocyte apoptosis, liver sinusoidal endothelial cell apoptosis, hepatocyte viability, liver sinusoidal endothelial viability, immune cell recruitment, macrophage activation, free radical generation, mitochondrial damage, pro-inflammatory compounds, albumin release, urea release and bile duct canaliculi. In one embodiment, the at least one liver disease biomarker is a non-alcoholic liver disease biomarker. In one embodiment, said at least one liver disease biomarker comprises inflammation, liver cell damage and steatohepatitis. In one embodiment, said hepatocyte layer is encased within an extracellular membrane layer.

In one embodiment, the present invention contemplates a method, comprising: a) providing; i) a microfluidic device comprising a solid substrate, said solid substrate comprising a membrane, one or more microfluidic channels and hepatic cells; ii) a physiological buffer solution comprising ethanol; b) contacting said hepatic cells with said physiological buffer solution under conditions that induces at least one stage of alcoholic liver disease in said hepatic cells; and c) detecting at least one alcoholic liver disease biomarker in said hepatic tissue. In another embodiment, the present invention contemplates a method, comprising: a) providing; i) a solid substrate comprising a single microfluidic channel; ii) a porous membrane separating said single microfluidic channel; iii) a hepatic tissue comprising human cellular architecture attached to said porous membrane; and iv) a physiological buffer solution comprising ethanol; b) contacting said hepatic tissue with said physiological buffer solution under conditions that induces at least one stage of alcoholic liver disease in said hepatic tissue; and c) detecting at least one alcoholic liver disease biomarker in said hepatic tissue. In one embodiment, said contacting comprises delivery of said ethanol at different concentrations. In one embodiment, said contacting comprises delivery of said ethanol at different frequencies. In one embodiment, said contacting comprises delivery of said ethanol at different durations. In one embodiment, said at least one alcoholic liver disease stage is selected from fatty liver tissue, alcoholic steatohepatitis, liver fibrosis, liver cirrhosis and hepatic carcinoma. In one embodiment, said at least one alcoholic liver disease biomarker is selected from the group consisting of lipid droplets, cytochrome P450 induction, hepatocyte apoptosis, liver sinusoidal endothelial cell apoptosis, hepatocyte viability, liver sinusoidal endothelial viability, free radical generation, mitochondrial damage, endoplasmic reticulum stress, pro-inflammatory compounds, albumin release, urea release and bile duct canaliculi. In one embodiment, the method comprises further providing an inlet channel and an outlet channel in fluidic communication with said single microfluidic channel. In one embodiment, inlet channel delivers said physiological buffer solution to said first and second chambers. In one embodiment, said outlet channel removes said physiological buffer solution from said first and second chambers. In one embodiment, the method further comprises flowing said physiological buffer solution into said first and second chambers with said inlet channel. In one embodiment, the method further comprises flowing said physiological buffer solution out of said first and second chambers with said outlet channel.

In one embodiment, the present invention contemplates a method, comprising: a) providing; i) a solid substrate comprising a single microfluidic channel; ii) a porous membrane separating said single microfluidic channel; iii) a hepatic tissue comprising human cellular architecture attached to said porous membrane and exhibits at least one non-alcoholic liver disease biomarker; and iv) a physiological buffer solution comprising a test compound; and b) contacting said hepatic tissue with said physiological buffer solution under conditions that the level of said at least one non-alcoholic liver disease biomarker is reduced. In one embodiment, said hepatic tissue is derived from a patient exhibiting at least one symptom of a disease selected from obesity, metabolic syndrome and/or type 2 diabetes. In one embodiment, the method comprises further providing an inlet channel and an outlet channel in fluidic communication with said single microfluidic channel. In one embodiment, inlet channel delivers said physiological buffer solution to said first and second chambers. In one embodiment, said outlet channel removes said physiological buffer solution from said first and second chambers. In one embodiment, the method further comprises flowing said physiological buffer solution into said first and second chambers with said inlet channel. In one embodiment, the method further comprises flowing said physiological buffer solution out of said first and second chambers with said outlet channel.

In one embodiment, the present invention contemplates a method, comprising: a) providing; i) a microfluidic device comprising a solid substrate, said solid substrate comprising a membrane, one or more microfluidic channels and hepatic cells; ii) a fluid comprising a concentration of fatty acid; b) contacting said hepatic cells with said fluid under conditions that induces at least one stage of non-alcoholic liver disease in said hepatic cells; and c) detecting at least one non-alcoholic liver disease biomarker in said hepatic tissue. In one embodiment, said detecting of step c) comprises detecting lipid accumulation. In one embodiment, the method further comprises adding immune cells to said hepatic cells.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

The term “alcohol” and “ethanol” as used herein, refer to an organic molecule having two carbon atoms, one of which is attached to a hydroxyl group. The terms may be used interchangeably and relate to the intoxicating substance in common beverages including, but not limited to, beer, wine and distilled spirits.

The term “alcoholic liver disease” as used herein, refers to the progressive damage and degeneration of hepatic tissue in stages including, but not limited to, fatty liver, alcoholic steatohepatitis (ASH), liver fibrosis and/or liver cirrhosis that may progress into the development of hepatocellular carcinoma.

The term “microfluidic” as used herein, relates to components where moving fluid is constrained in or directed through one or more channels wherein one or more dimensions are 1 mm or smaller (microscale). Microfluidic channels may be larger than microscale in one or more directions, though the channel(s) will be on the microscale in at least one direction. In some instances the geometry of a microfluidic channel may be configured to control the fluid flow rate through the channel (e.g. increase channel height to reduce shear). Microfluidic channels can be formed of various geometries to facilitate a wide range of flow rates through the channels.

The term “channels” as used herein, are pathways (whether straight, curved, single, multiple, in a network, etc.) through a medium (e.g., silicon) that allow for movement of liquids and gasses. Channels thus can connect other components, i.e., keep components “in communication” and more particularly, “in fluidic communication” and still more particularly, “in liquid communication.” Such components include, but are not limited to, liquid-intake ports and gas vents. Microchannels are channels with dimensions less than 1 millimeter and greater than 1 micron.

It is not intended that the present invention be limited to particular microfluidic device designs. A variety of designs is contemplated, including those found in U.S. Pat. No. 8,647,861, hereby incorporated by reference.

The phrases “connected to,” “coupled to,” “in contact with” and “in communication with” as used herein, refer to any form of interaction between two or more entities, including mechanical, electrical, magnetic, electromagnetic, fluidic, and thermal interaction. For example, in one embodiment, channels in a microfluidic device are in fluidic communication with cells and (optionally) a fluid source such as a fluid reservoir. Two components may be coupled to each other even though they are not in direct contact with each other. For example, two components may be coupled to each other through an intermediate component (e.g. tubing or other conduit).

The term “solid substrate” as used herein, refers to a substrate that may be biological, nonbiological, organic, inorganic, or a combination of any of these, existing as particles, strands, precipitates, gels, sheets, tubing, spheres, containers, capillaries, pads, slices, films, plates, slides, etc. The solid substrate is preferably flat but may take on alternative surface configurations. For example, the solid substrate may contain raised or depressed regions, such as microfluidic channels and/or inlet and outlet ports. For example, the substrate may be functionalized glass, Si, Ge, GaAs, GaP, SiO₂, SiN₄, modified silicon, nitrocellulose and nylon membranes, or any one of a variety of gels or polymers such as (poly)tetrafluoroethylene, (poly)vinylidendifluoride, polystyrene, polycarbonate, or combinations thereof. Other suitable solid substrate materials are be readily apparent to those of skill in the art. The surface of the solid substrate may also contain reactive groups, which could be carboxyl, amino, hydroxyl, thiol, or the like. More preferably, the surface will be optically transparent and will have surface Si—OH functionalities, such as are those found on silica surfaces.

The term “porous membrane” as used herein, refers to a material that is flexible. elastic, or a combination thereof with pores large enough to only permit exchange of gases and small chemicals, or large enough to permit migration and transchannel passage of large proteins, and/or portions thereof. The membrane may also be designed or surface patterned to include micro and/or nanoscopic patterns therein such as grooves and ridges, whereby any parameter or characteristic of the patterns may be designed to desired sizes, shapes, thicknesses, filling materials, and the like.

The term “chamber” as used herein, refers to an isolated region of a microchannel that is separated by a porous membrane. For example, the porous membrane may extend longitudinally down the midpoint of a microchannel thereby providing an upper chamber and a lower chamber.

The term “human cellular architecture” as used herein, refers to a spatial and temporal organization of cells that have biochemical interactions that support a human tissue-like function. Such architecture may be reflected in a naturally occurring tissue, but also as a result of maturation and differentiation process that occur during the use of the microfluidic tissue testing system as disclosed herein. For example, a hepatic human cellular architecture residing within a microchannel of the tissue testing device includes, but is not limited to, interacting hepatocytes, Kupffer cells, stellate cells and/or blood cells that result in naturally occurring functions of in vivo hepatic tissue.

The term “biomarker” as used herein, refers to a distinctive biological or biologically derived indicator (as a biochemical metabolite in the body) of a process, event, or condition (for example, a particular stage of ALD).

The term “hepatocyte layer” as used herein, refers to any of the polygonal epithelial parenchymatous cells of the liver that secrete bile that are configured to maintain cell-cell contact and fluid/biochemical communication.

The term “endothelial cell layer” as used herein, an epithelium of mesoblastic origin composed of thin flattened cells that lines internal body cavities, such as liver bile ducts. Specific endothelial cells may include, but are not limited to, Kupffer cells and/or stellate cells bile that are configured to maintain cell-cell contact and fluid/biochemical communication.

The term “ blood vessel cell layer” as used herein, refers to at least one cell layer including, but not limited to: i) a layer of simple squamous endothelial cells embedded within a polysaccharide intercellular matrix, surrounded by a thin layer of subendothelial connective tissue interlaced with a number of circularly arranged elastic bands; ii) a layer of circularly arranged elastic fiber, connective tissue, polysaccharide substances, that may be rich in vascular smooth muscle cells; and ii) a layer of connective tissue that may also contain nerve cells.

The term “physiological buffer solution” as used herein, refers to a solution that usually contains on the one hand either a weak acid (as carbonic acid) together with one of the salts of this acid or with at least one acid salt of a weak acid or on the other hand a weak base together with one of the salts of the base and that by its resistance to changes in hydrogen-ion concentration on the addition of acid or base is useful in many chemical, biological, and technical processes. Buffer solutions maintained at a pH of approximately 7.4 are preferred, of which, many types and chemical compositions are known in the art. Such buffer solutions may also be designed to provide solubility for drugs, toxic agents (e.g., ethanol) and/or peptides and proteins. Solubility parameters may be modified by the addition of non-toxic solvents including, but not limited to, dimethylsulfoxide and/or polyethylene glycol.

The term “induces”, “inducing” or “induced” as used herein, refers to a mechanism that causes, or brings about, a physiological response to the presence of a compound. For example, the exposure of ethanol to hepatic tissue may trigger mechanisms as described herein that induce stages and symptoms of ALD.

The term “delivers”, “delivering” or “delivered” as used herein, refers to the movement of a compound from one location to another. For example, the microfluidic tissue testing system may include an inlet port the provides for the movement of a physiological buffer comprising a compound from an inlet port to a hepatic tissue having a human cellular architecture. Such movement may be accomplished by a fluid flow through one or more channels.

The term “different concentrations” as used herein, refers to a tissue testing regimen that compares data collected from tissues that are exposed to a physiological buffer solution comprising a compound having a concentration range of low to high for equal period of time. For example, a range of physiologically relevant concentrations of ethanol may be compared, between approximately 5-20 mM.

The term “different frequencies” as used herein, refers to a tissue testing regimen that compares data collected from tissues that are repeatedly exposed to a physiological buffer solution comprising a compound having unequal period of times between each exposure.

The term “different durations” as used herein, refers to a tissue testing regimen that compares data collected from tissues that are repeatedly exposed to a physiological buffer solution comprising a compound having unequal periods of time for each exposure.

The term “disease” or “medical condition”, as used herein, refers to any impairment of the normal state of living tissue from an animal or plant body or one of its parts that interrupts or modifies the performance of the vital functions. Typically manifested by distinguishing signs and symptoms, it is usually a response to: i) environmental factors (as malnutrition, a hypercaloric diet, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any response in an untreated cell or tissue relative to a treated cell or tissue, mean that the quantity and/or magnitude of the response in the treated cell or tissue is lower than in the untreated cell or tissue by any amount that is recognized as biologically relevant by any scientifically trained personnel. In one embodiment, the quantity and/or magnitude of the response in the treated cell or tissue is at least 10% lower than, at least 25% lower than, at least 50% lower than, at least 75% lower than, and/or at least 90% lower than the quantity and/or magnitude of the response in the untreated cell or tissue.

The term “fibrotic” as used herein, refers to any tissue response marked by an increase of interstitial fibrous tissue.

The term “cirrhosis” as used herein, refers to a widespread disruption of normal liver structure by fibrosis and the formation of regenerative nodules that is caused by any of various chronic progressive conditions affecting the liver such as long-term alcohol abuse, long-term non-alcohol steatohepatitis, or hepatitis.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like. A drug is attached to a medium (or carrier) if it is impregnated, incorporated, coated, in suspension with, in solution with, mixed with, etc.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “test compound” as used herein, refers to any compound or molecule considered a candidate as an inhibitory compound.

The term “protein” as used herein, refers to any of numerous naturally occurring extremely complex substances (as an enzyme or antibody) that comprise amino acid residues joined by peptide bonds, contain the elements carbon, hydrogen, nitrogen, oxygen, usually sulfur. In general, a protein comprises amino acids having an order of magnitude within the hundreds.

As used herein, the terms “siRNA” refers to either small interfering RNA, short interfering RNA, or silencing RNA. Generally, siRNA comprises a class of double-stranded RNA molecules, approximately 20-25 nucleotides in length. Most notably, siRNA is involved in RNA interference (RNAi) pathways and/or RNAi-related pathways. wherein the compounds interfere with gene expression.

As used herein, the term “shRNA” refers to any small hairpin RNA or short hairpin RNA. Although it is not necessary to understand the mechanism of an invention, it is believed that any sequence of RNA that makes a tight hairpin turn can be used to silence gene expression via RNA interference. Typically, shRNA uses a vector stably introduced into a cell genome and is constitutively expressed by a compatible promoter. The shRNA hairpin structure may also cleaved into siRNA, which may then become bound to the RNA-induced silencing complex (RISC). This complex binds to and cleaves mRNAs which match the siRNA that is bound to it.

As used herein, the term “microRNA”, “miRNA”, or “μRNA” refers to any single-stranded RNA molecules of approximately 21-23 nucleotides in length, which regulate gene expression. miRNAs may be encoded by genes from whose DNA they are transcribed but miRNAs are not translated into protein (i.e. they are non-coding RNAs). Each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIG. 1A-E demonstrates the cellular architectural similarity between in vivo hepatic tissue and one embodiment of a microfluidic liver testing system. Data shown was collected after 10 days in culture but similar trends were seen for ˜30 days (not shown). These data validate cell competency in creating an in vitro physiologically relevant system.
FIG. 1A: A representative schematic of liver sinusoid architecture in vivo.
FIG. 1B: A representative schematic of a microfluidic tissue testing system constructed without Kupffer cells.
FIG. 1C: A representative schematic of a microfluidic tissue testing system constructed with Kupffer cells.
FIG. 1D: Exemplary data showing the functionality and competency of the microfluidic liver testing system. Panels read left to right: fluorescent dye pumped into active bile canaliculi between adjacent primary human hepatocytes (PHHs); CD31 staining in the cultured liver sinusoidal endothelial cells (LSECs); CD68 staining of the KCs.
FIG. 1E: Exemplary data showing that CYP3A4 activity in parenchymal/non-parenchymal co-cultures within a microfluidic tissue testing system is higher than in hepatocyte monolayers plated in a static well.

FIG. 2A-B presents exemplary data from a microfluidic tissue testing system showing maintenance of fatty liver phenotypes on a high-fat diet (FIG. 2B) versus a normal fat diet (FIG. 2A). Hepatocytes stained with Nile Red demonstrated an increased accumulation of lipid droplets (FIG. 2B) than control (FIG. 2A). Under a high-fat diet fatty liver phenotypes also exhibited a significant increase of free fatty acid, cholesterol and glucose, as well as mitochondrial damage and oxidative stress increase.

FIG. 3A-C presents one embodiment of a microfluidic tissue testing system.
FIG. 3A: Schematic of a microfluidic channel comprising a solid substrate flanked by inlet and outlet channels, wherein the microfluidic channel comprises a top chamber parenchymal hepatocyte layer encased in extracellular membrane and a bottom chamber non-parenchymal support cell layer. The arrows indicate the direction of fluid flow through the top and bottom chambers of the microchannel.
FIG. 3B: Phase contrast image of primary human hepatocytes (PHHs) in a collagen sandwich after 10 days of culture.
FIG. 3C: Phase contrast image of LSECs after 10 days in culture.

FIG. 4A-B presents exemplary photomicrograph data demonstrating hepatic toxicity using a 30 μM CPD-N treatment on hepatocyte microchip hepatic tissue. All assessments were made in the presence of NucBlue to monitor chromosomal integrity.
FIG. 4A: Deformed mitochondrial membrane potential marker (TMRM (red) and increased hepatic oxidative stress as measured by CellRox dye (blue).
FIG. 4B: Decreased bile canaliculi structures as measured by (5, 6)-carboxy-2′,7′-dichlorofluorescein diacetate (carboxy-DCFDA) dye (green)

FIG. 5A-B presents exemplary photomicrograph data demonstrating hepatic toxicity using a 10 μM CPD-N treatment on hepatocyte microchip hepatic tissue. All assessments were made in the presence of NucBlue to monitor chromosomal integrity.
FIG. 5A: Increased steatosis as measured by an AdipoRed dye (red).
FIG. 5B: Activated stellate cells as measured by aSMA expression.

FIG. 6A-C presents exemplary data of a dose-dependent relationship of CPD-N induced hepatic toxicity on a hepatocyte microchip.
FIG. 6A: Mitochondrial deformation.
FIG. 6B: Reactive oxygen species production.
FIG. 6C: Bile canaliculi abundance.

FIG. 7A-B presents exemplary data of liver tissue disease phenotype development subsequent to exposure to a high oleic acid concentration media.
FIG. 7A: Increased concentration of lipid droplets in hepatocytes.
FIG. 7B: Left, Control. Right, High fat diet (oleic acid; 5 μM) 2 day exposure. Morphological features indicative of injury/stress exposure in LSEC cells. BF (bright field). DAPI, blue.

FIG. 8 presents one embodiment of a hepatic steatosis pathway.

FIG. 9 presents one embodiment of a hepatic inflammatory pathway.

FIG. 10 presents exemplary data showing hepatocyte co-cultures on a hepatocyte microchip with a plurality of cells, including, but not limited to, immune cells, LSEC cells, Kupffer cells and/or stellate cells.

FIG. 11 presents exemplary data of long-term culture (e.g., 14 days) of either hepatocytes or LSEC cells from human, dog or rat cells showing proper maintenance of in vivo-like morphologies.

FIG. 12 presents exemplary data comparing conventional plate cell culture with a hepatocyte microchip platform for long-term culture (e.g., 14 days) of either hepatocytes or LSEC cells from human, dog or rat cells showing proper maintenance of a drug transporter protein. Multidrug resistance-associated protein 2 (MRP2) staining correctly showed in vivo polarization of the transporter at the canalicular membrane, conventional plates cultured showed only background staining of dead cells, indicative of a lack of transporter expression or polarization.

FIG. 13A-B presents exemplary data showing a quadruple co-culture of hepatocytes, Kupffer cells, LSEC cells and Stellate cells.
FIG. 13A: Kupffer cell activation with lipopolysaccharide (LPS) (Phagocytosis of microbeads, red).
FIG. 13B: Stellate cell activation with TGF-β (α-SMA staining, green).

FIG. 14 presents an illustration of the progressive nature of liver disease through the stages of NALFD, NASH and liver cirrhosis.

FIG. 15A-B presents exemplary data of an in vitro NAFLD model showing the accumulation of fat droplets in co-cultured of hepatocytes and LSECs on a hepatocyte microchip after two days of exposure to a high fat diet of oleic acid. Hepatocyte fat droplet staining with Nile Red® and AdipoRed® (red). LSEC morphology was determined by staining with 4-(2-di-n-propylaminoethyl)indole (DAPI: blue).
FIG. 15A: Control hepatocyte lipid (fat) droplets.
FIG. 15B: hepatocyte fat droplet accumulation at 24 hours of high fat oleic acid administration.

FIGS. 16A-C present exemplary data showing a two day time course of accumulating hepatocyte fat droplets in a co-culture with LSECs on a hepatocyte microchip in accordance with FIG. 15. Nile Red® and AdipoRed® (red); Nuclear staining 4-(2-di-n-propylaminoethyl)indole (DAPI: blue).
FIG. 16A: Control hepatocyte fat droplets.
FIG. 16B: hepatocyte fat droplet accumulation at 24 hours of high fat oleic acid administration.
FIG. 16C: Hepatocyte fat droplet accumulation after 48 hours of high fat oleic acid administration.

FIGS. 17A-B present exemplary data showing the number of hepatocyte fat droplet accumulation and glucose levels in accordance with FIG. 15.
FIG. 17A: A scatterplot presentation of the number of lipid droplets within the hepatocytes after 48 hours of high fat diet exposure. (lipid droplets (n per image))
FIG. 17B: A scatterplot presentation of extracellular glucose levels after 48 hour of high fat diet exposure.

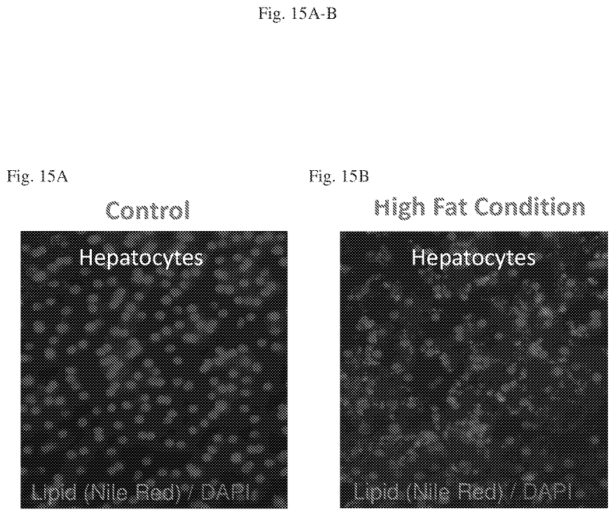

FIG. 18A-H present exemplary data showing effects on cell metabolism in accordance with FIG. 15. FIG. 18A: Cell Viability and mitochondrial function; FIG. 18B-F: Oxidative Stress; and FIG. 18D-H Fatty Acid content.
FIG. 18A: Determination of mitochondrial function as an indicator of cell viability. Tetramethylrhodamine, methyl ester staining followed by image analysis.
FIG. 18B: Determination of intracellular reactive oxidative species (ROS) as determined by CellRox® followed by image analysis quantification.
FIG. 18C: Determination of mitochondrial function in Hepatocytes (top) LSECs (bottom) after 24 hours of a high fat diet. Control, left panels, High Fat Diet, right panels. Red Stain: Tetramethylrhodamine, methyl ester staining (membrane potential active mitochondria stain); Blue Stain: DAPI
FIG. 18D: Determination of mitochondrial function in hepatocytes after 24 hours (control top panels) of a high fat diet (bottom panels). Red Stain: Tetramethylrhodamine, methyl ester staining.
FIG. 18E: Determination of mitochondrial function in LSECs after 48 hours (control left) of a high fat diet (right). Red Stain: Red Stain: Tetramethylrhodamine, methyl ester staining; Blue Stain: DAPI.
FIG. 18F: Determination of mitochondrial function in hepatocytes after 48 hours (control top panels) of a high fat diet (bottom panels). Red Stain: Red Stain: Tetramethylrhodamine, methyl ester staining.
FIG. 18G: Graphical Determination of extracellular free fatty acids.
FIG. 18H: Graphical Determination of extracellular cholesterol.

FIG. 19 presents exemplary data showing an increase in hepatocyte cell nuclei in accordance with FIG. 15.

FIGS. 20A-D present exemplary data showing an in vitro NASH model in a tri-culture comprising hepatocytes, LSECs and Kupffer cells during exposure to a high fat diet comprising oleic acid and, optionally, LPS.
FIG. 20A: Control: No oleic acid/No LPS.
FIG. 20B: Oleic acid only.
FIG. 20C: Oleic acid followed by LPS.
FIG. 20D: LPS only.

FIG. 21A-B presents exemplary data showing scatterplot data from the tri-culture in vitro NASH model in accordance with FIG. 20.
FIG. 21A: Quantitation of intracellular lipid (fat) droplets.
FIG. 21B: Quantitation of extracellular glucose.

FIG. 22A-B presents exemplary data showing a IF AdipoRed stained tri-culture in vitro NASH model of hepatocyte fat droplet density as a function of hepatocyte microchip channel location in accordance with FIG. 20.
FIG. 22A: hepatocyte lipid (fat) droplet density proximal to channel outlet.
FIG. 22B: hepatocyte lipid (fat) droplet density proximal to channel inlet.

FIG. 23 presents an illustration of one embodiment of an in vivo liver architecture showing the differentiation of hepatocytes into “metabolic zones” based upon a proximal location to the central vein (CV) (e.g., downstream pericentral) or the portal blood tract (PT) (e.g., upstream periportal).

FIGS. 24A-B compare the normal in vivo liver tissue architecture to an in vitro quad-culture grown in the presently disclosed hepatocyte microchip.
FIG. 24A shows a schematic illustration of exemplary cellular components in a healthy liver.
FIG. 24B shows a schematic illustration of one embodiment of a liver-chip showing a quadruplicate culture comprising hepatocytes (HEP) green square outlined in blue; HSC (red); membrane (green) separating the upper channel from LSEC (grey rectangles) and Kupffer Cells (KC) in the lower channel.

FIG. 25 presents exemplary photomicrographs of the quad-culture cells within the presently disclosed hepatocyte microchip.

FIG. 26A-D presents exemplary photomicrographs of HSC cell growth in a variety of extracellular membrane matrices.
FIG. 26A: Matritek® with no HSCs+Matrigel® overlay.
FIG. 26B: Matritek® with HSCs+Matrigel® overlay.
FIG. 26C: Matritek® with HSCs+Matrigel® overlay.
FIG. 26D: Collagen with HSCs+Matrigel® overlay.

FIG. 27A-B presents exemplary photomicrographs of mouse in vivo HSC bile canaliculi, CD31 positive (FIG. 27A) and sinusoids, Flk1 positive (FIG. 27B).

FIGS. 28A1-D1 and 28A2-D2 presents exemplary photomicrographs of in vitro quad-culture NASH model comprising hepatocytes with differentiated HSC bile canaliculi (FIG. 28A1-D1 and FIG. 28A2-D2) in a variety of extracellular membrane matrices.
FIG. 28A1 and FIG. 28A2 (higher power image): Matritek® with no HSCs+Matrigel® overlay.
FIG. 28B1 and FIG. 28B2 (higher power image): Matritek with HSCs+Matrigel® overlay.
FIG. 28C1 and FIG. 28C2 (higher power image): Matrigel® with HSCs+Matrigel® overlay.
FIG. 28D1 and FIG. 28D2 (higher power image): Collagen with HSCs+Matrigel® overlay.

FIG. 29 presents a representative timeline for the performance of a cell culture protocol using a hepatocyte microchip. Exemplary endpoints: morphology and sample collection for measuring albumin, cholesterol, and glucose quantification. Flow rate: 30 ul/hour.

FIG. 30 presents representative photomicrographs of LSECs incubated for seven days in a variety of cell culture media showing their viability. Advanced DMEM/F12: WEM+EGM2 (upper row); Endothelial media (e.g. Cell Systems) (middle row); Endothelial media: WEM (lower row). From upper to lower panel, left to right: 0% FBS+cholesterol; 0% FBS; 1% FBS; 2% FBS; 2% FBS; 1% FBS; 0% FBS; 0% FBS+cholesterol; 2% FBS; 1% FBS; 0% FBS; and 0% FBS +cholesterol.

FIGS. 31A-B present exemplary data showing the viability of rat LSEC and hepatocytes after 6 days of incubation in a 10%WEM (FIG. 32A) vs. 2% FBS (FIG. 32B) culture media.

FIG. 32 presents one embodiment of a hepatocyte microchip protocol to test the effects of fructose on lipid droplet accumulation. Day −1 coating, Day 0 hepatocyte seeding, Day 1 Matrigel overlay, Day 2 LSEC seeding, Day 3 connection to flow, Day 6 dose with glucose: fructose, dose with intestine effluent (1:1).

FIG. 33A-B presents exemplary photomicrographs of hepatocytes after 48 hours incubation in a liver culture media (control-CTL, left panel) comparing low fructose (middle panel) and high fructose (left panel) concentration.
FIG. 33A: Hepatocyte morphology.
FIG. 33B: Hepatocyte lipid staining (AdipoRed). Nuclei: DAPI, blue.

FIG. 34A-B presents exemplary photomicrographs of hepatocytes after 72 hours incubation in an intestinal effluent culture media (control-CTL, left panel) comparing low fructose (middle panel) and high fructose (left panel) concentration.
FIG. 34A: Hepatocyte morphology.
FIG. 34B: Hepatocyte lipid staining (AdipoRed). Nuclei: DAPI, blue.

FIG. 35 presents exemplary data showing the effect of fructose on triglycerides after 48 h hours of incubation compared to control (CTL).

FIG. 36 shows exemplary data of hepatic lipid (fat) accumulation in response to ethanol, high fat diet, or drug exposure (e.g., Cpd 6) as a biomarker of either alcoholic or non-alcoholic liver disease development, or drug-induced steatosis.

FIG. 37A-D shows exemplary data of Hepatocytes Polyploidy cell proliferation (FIG. 37A-B) or glucose levels (FIG. 37C-D) in response to ethanol (FIG. 37D), high fat diet (FIG. 37B-C) or drug exposure (e.g., Cpd 2) (FIG. 37A) as a biomarker of either alcoholic or non-alcoholic liver disease development.

FIGS. 38A-B show exemplary data of cholesterol (ug/ml) (FIG. 38A), or reactive oxygen species (ROS-number (n) of events) (FIG. 38B), in response to a high fat diet (FIG. 38B) or drug exposure (e.g., Cpd 5, 20 uM) (FIG. 38A) as a biomarker of either alcoholic or non-alcoholic liver disease development.

FIG. 39A-B demonstrates the development of bile canaliculi in the presently disclosed hepatocyte microchip after three (3) days of culture.
FIG. 39A: Control microphotograph, phase contrast.
FIG. 39B: Canaliculi transporter stain 2′,7′-dichlorodihydrofluorescein diacetate (CDFDA) (green) fluorescent microphotograph.

FIG. 40A-B presents exemplary data showing MRP2 expression in bile caniculi after fourteen (14) days of culture.
FIG. 40A: MRP2 expression in hepatocytes cultured in a conventional plated sandwich culture.
FIG. 40B: MRP2 expression in one embodiment of the presently disclosed hepatocyte (liver) microchip. Scale bar=50 micrometer.

FIG. 41A-B presents exemplary data showing the superior release of hepatocyte biomarkers of albumin secretion (m/day/million cells) (FIG. 41A) and urea production (m/day/million cells) (FIG. 42B) in the presently disclosed microchip (blue line) as opposed to conventional static cell culture technology (red line).

FIG. 42 presents CYP activity in hepatocyte Liver-Chip (grey bars) as compared to conventional static cell culture technology (black bars). Suspension hepatocytes as a positive control (day zero). Compared are data from CYP1A, CYP2B and CYP3A from human, rat and dog.

FIG. 43 presents exemplary data showing the positive correlation between LPS-stimulated TNF-α release (10 μg/ml LPS for 24 hours) and the Kupffer Cell: Hepatocyte ratio (K:H) after a ten (10) day culture. *=undetectable.

FIG. 44A-B presents exemplary data showing the accumulation of cytokines (IL-1a, IL-1b and IL-6) in both the hepatocyte channel (FIG. 44A) and the LSEC/Kupffer cell channel (FIG. 44B) of the presently disclosed hepatocyte microchip.

FIG. 45 presents exemplary data and a schematic showing changes in hepatocyte cellular architecture as a result of the development of fibrosis.

FIG. 46A-B demonstrates the development of hepatocyte fibrosis.
FIG. 46A: A schematic of the cellular architecture of hepatocyte fibrotic tissue in the Liver-Chip
FIG. 46B: A photomicrograph showing the activation of hepatic stellate cells by TGF-α during the development of fibrosis in the Liver-Chip.

FIG. 47A-B presents exemplary data showing superior CYP450 activity and expression in hepatocyte microchip cultures (e.g., co-culture, tri-culture and quad-culture) as compared to conventional cell culture conditions.
FIG. 47A: CYP3A4 enzyme activity.
FIG. 47B: CYP3A4 gene expression

FIG. 48A-B presents exemplary data showing an improved CPD-K fibrotic activation effect (hSC activation) on human stellate cells cultured in a gel-based quad-culture microchip following exposure to JNJK at concentrations of 3 μM, 10 μM and 50 μM.
FIG. 48A cells immunostained with alphaSMA (pink) and nuclei blue (DAPI).
FIG. 48B chart showing increase in activated stellate cells (normalized intensity).

FIG. 49A-B presents exemplary data showing the release of alanine transaminase (ALT) in response to various concentrations of bosentan.
FIG. 49A: Human hepatocyte microchip culture.
FIG. 49B: Human hepatocyte conventional plate cell culture.

FIG. 50A-B presents exemplary data showing the effect of bosentan on co-transporter gene expression.
FIG. 50A: Sodium/bile co-transporter.
FIG. 50B: Bile Salt Export Pump (BSEP, ABCB11) co-transporter.

FIG. 51A-B presents exemplary data showing LSEC viability determinations by Ac LDL uptake in the comparison of media.
FIG. 51A: 10% CSC media.
FIG. 51B: AdDMEMF12+EGM-2 media.

FIG. 52 presents exemplary data showing cell confluency in several media after co-culturing LSECs (upper channel) and hepatocytes (lower channel) for seven (7) days. LSEC: CSC2%; LSEC: AdMEM:WEM (1:1); LSEC: AdMEM; HEP:WEM-2% (lower row).

FIG. 53 presents exemplary data showing LSEC cell viability in nine (9) different cell culture media (infra). Panels 1-9, upper left (1) to lower left (9).

FIG. 54A-D presents exemplary data comparing hepatic stellate cell morphology in a 2D cell culture and a 3D cell culture.
FIG. 54A: One embodiment of a conventional two dimensional (2D) plate cell culture. Phase contrast micrograph.
FIG. 55B: One embodiment of a microfluidic microchip channel three dimensional (3D) cell culture. Phase contrast micrograph.
FIG. 55C: One embodiment of a conventional cell 2D culture plate. Alpha SMA (hepatic stellate cell activation marker) staining in green. DAPI, blue.
FIG. 55D: One embodiment of a 3D hepatocyte microchip. Alpha SMA (hepatic stellate cell activation marker) staining in green. DAPI, blue.

FIG. 55A-C presents several embodiments of a hepatocyte tri-culture.
FIG. 55A: ECM/Matrigel® where the ECM molecular coating is located under the hepatocytes and the Matrigel® molecular coating is located above the hepatocytes.
FIG. 55B: ECM/3D gel overlay where the ECM molecular coating is located under the hepatocytes and a 3D gel overly (less than 100 μm) is located above the hepatocytes.
FIG. 55C: 3D gel underlay/3D gel overlay where a 3D gel underlay (under the hepatocytes) and a 3D gel overly (above the hepatocytes) is used. Hep (hepatocytes) grey squares; T (top-upper channel); B (bottom-lower channel); rectangles (LSEC); Kupffer cells (KC-blue stars).

FIG. 56A-C presents several embodiments of a hepatocyte quad-culture.
FIG. 56A: ECM/Matrigel® where the ECM molecular coating is located under the hepatocytes and the Matrigel® molecular coating is located above the hepatocytes.
FIG. 56B: ECM/3D gel overlay where the ECM molecular coating is located under the hepatocytes and a 3D gel overly (less than 100 μm) is located above the hepatocytes.
FIG. 56C: 3D gel underlay/3D gel overlay where a 3D gel underlay (under the hepatocytes) and a 3D gel overly (above the hepatocytes) is used. Hep (hepatocytes) grey squares; T (top-upper channel); B (bottom-lower channel); rectangles (LSEC); Kupffer cells (KC-blue stars); and HSC (red).

FIG. 57A-C presents exemplary data showing the accumulation of lipid (fat) droplets in cells (green) over an extended time period. DAPI, blue.
FIG. 57A: Control: 0 hours of high fat diet exposure.
FIG. 57B: 40 hours of high fat diet exposure.
FIG. 57C: 64 hours of high fat diet exposure.

FIG. 58 presents exemplary data showing elevated free fatty acids (nmol/well) in hepatocytes subsequence to forty (40) hours of high fat diet exposure. CTL-control 40 hours. Media.

FIG. 59A-C presents exemplary data showing lipid (fat) droplet accumulation in a high fat diet.
FIG. 59A: Liver-Chip hepatocyte lipid droplet accumulation in control (top panel), high fat diet (48 h) (middle panel) and high fat diet+Exendin (48 hours of high fat diet followed by 24 h exposure to Exendin treatment) (bottom panel).
FIG. 59B: Quantification of lipid droplet accumulation (n) data in control, high fat diet and high fat diet+Exendin Liver-Chip.
FIG. 59C: Quantification of cell nuclei (DAPI stain (n)) in control, high fat diet and high fat diet+Exendin Liver-Chip conditions.

FIGS. 60A-B present exemplary data showing the effect of Liver-Chip high fat diet and/or Exendin on glucose levels at both the 24 hour (FIG. 60A) and 48 hour (FIG. 60B) exposure time points.

FIG. 61A-C presents exemplary data showing the effect of 48 hour high fat diet exposure on reactive oxygen species (ROS) and functional mitochondria (TMRM).
FIG. 61A: CellRox® (ROS, oxidative stress marker) quantification in images from control Liver-Chip and high fat diet exposed Liver-Chip.
FIG. 61B: mitochondrial quantification by live imaging of chips stained with tetramethylrhodamine (TMRM).
FIG. 61C: DAPI stained nuclei quantification.

FIG. 62A-C presents exemplary data showing the effect of high fat diet on the spatial distribution pattern of hepatocyte lipid droplets in the inlet, middle and outlet of the microchip after 48 hours of high fat diet exposure.
FIG. 62A: Control. No high fat diet, no Exendin.
FIG. 62B: High fat diet only.
FIG. 62C: High fat diet plus Exendin.

FIG. 63 presents an illustration of the study plan timeline for the administration of physiological concentrations of ethanol to a heptaocyte tri-culture with Kupffer cells. The End Point arrows indicate days where the various cellular parameters were measured.

FIG. 64A-B presents exemplary microchannel photomicrographs of control hepatocyte/LSEC cell layer morphological data before connection of the microchip to the perfusion fluid (BF Pre-connection) (e.g., before day 0 of FIG. 63).
FIG. 643A: Hepatocytes Top microchannel. Outlet region (left micrograph). Mid-region (middle micrograph). Inlet region (right micrograph).
FIG. 64B: LSEC Bottom microchannel. Outlet region (left micrograph). Inlet region (right micrograph).

FIG. 65A-B presents exemplary microchannel photomicrographs of control hepatocyte/LSEC cell layer morphological data after connection of the microchip to the perfusion fluid but before ethanol dosing (BF Pre-dosing)(e.g., before Day 0 of FIG. 63).
FIG. 65A: Top microchannel. Outlet region (left micrograph). Mid-region (middle micrograph). Inlet region (right micrograph).
FIG. 65B: Bottom microchannel. Outlet region (left micrograph). Inlet region (right micrograph).
FIG. 66A-B presents exemplary top microchannel photomicrographs of treated hepatocyte cell layer morphological data during twenty-four (24) hours of perfusion with various physiological ethanol concentration dosing (e.g., Day 1 of FIG. 63). FIG. 66A: Top Left: 0.04%; Bottom Left: 0.16%. FIG. 66B: Top Right: 0.08%; Bottom right: Vehicle.

FIG. 67 presents exemplary top microchannel photomicrographs of treated hepatocyte cell layer morphological data after twenty-four (24) hours of perfusion with various physiological ethanol concentration dosing (e.g., Day 1 of FIG. 63). Top Left: Vehicle. Top Right: 0.04%; Bottom Left: 0.08%. Bottom Right: 0.16%.

FIG. 68 presents exemplary top microchannel photomicrographs of treated hepatocyte cell layer morphological data after forty-eight (48) hours of perfusion with various physiological ethanol concentration dosing (e.g., Day 2 of FIG. 63). Top Left: Vehicle. Top Right: 0.04%; Bottom Left: 0.08%. Bottom Right: 0.16%.

FIG. 69 presents exemplary top microchannel photomicrographs of treated hepatocyte cell layer morphological data forty-eight (48) hours after perfusion with various physiological ethanol concentrations was discontinued (e.g., Day 4 of FIG. 63). Top Left: Vehicle. Top Right: 0.04%; Bottom Left: 0.08%. Bottom Right: 0.16%.

FIG. 70 presents exemplary top microchannel photomicrographs of treated hepatocyte cell layer morphological data one hundred and twenty (120) hours after perfusion with various physiological ethanol concentrations was discontinued (e.g., Day 7 of FIG. 63). Top Left: Vehicle. Top Right: 0.04%; Bottom Left: 0.08%. Bottom Right: 0.16%.

FIG. 71A-C presents exemplary data of lipid droplet accumulation subsequent to forty-eight (48) hours of perfusion with various physiological ethanol concentrations (vehicle, 0.04%, 0.08% and 0.16%) (e.g., Day 2 of FIG. 63).
FIG. 71A: Ethanol dosing for 48 h. Lipid droplet accumulation per image.
FIG. 71B: Ethanol dosing for 48 h. Lipid droplet accumulation per hepatocyte cell (n/nuclei).
FIG. 71C: After Recovery. Lipid droplet accumulation per hepatocyte cell, n/nuclei.

FIG. 72A-B presents exemplary data of lipid droplet accumulation one hundred and twenty hours after perfusion (i.e. recovery) with various physiological ethanol concentrations was discontinued (e.g., Day 7 of FIG. 63). Data presented at 0.16% ethanol is statistically significant as compared to vehicle control.
FIG. 72A: Lipid droplet accumulation per photomicrograph image.
FIG. 72B: Lipid droplet accumulation per hepatocyte cell.

FIG. 73 presents exemplary data of cholesterol accumulation after perfusion with various physiological ethanol concentrations.

FIG. 74 presents exemplary data of glucose accumulation after perfusion with various physiological ethanol concentrations. After twenty-four hours (e.g., Day 1 of FIG. 63)

FIG. 75A-B presents exemplary data of glucose and glycogen accumulation after perfusion with various physiological ethanol concentrations was discontinued.
FIG. 75A: Forty-eight hours of recovery (e.g., Day 4 of FIG. 63)
FIG. 75B: Glycogen quantification from cell lysate. Indication of glycogen storage.

FIG. 76A-B presents exemplary data of triglyceride (TG) accumulation after perfusion with various physiological ethanol concentrations.
FIG. 76A: After twenty-four hours (e.g., Day 1 of FIG. 63)
FIG. 76B: After forty-eight hours (e.g., Day 2 of FIG. 63)

FIG. 77A-B presents exemplary data of triglyceride (TG) accumulation after perfusion with various physiological ethanol concentrations was discontinued.
FIG. 77A: Forty-eight hours of recovery (e.g., Day 4 of FIG. 63)
FIG. 77B: One hundred and twenty hours of recovery (e.g., Day 7 of FIG. 63)

FIG. 78A-B presents exemplary data of albumin accumulation after perfusion with various physiological ethanol concentrations.
FIG. 78A: After twenty-four hours (e.g., Day 1 of FIG. 63)
FIG. 78B: After forty-eight hours (e.g., Day 2 of FIG. 63)

FIG. 79A-B presents exemplary data of albumin accumulation after perfusion with various physiological ethanol concentrations was discontinued.
FIG. 79A: Forty-eight hours of recovery (e.g., Day 4 of FIG. 63)
FIG. 79B: One hundred and twenty hours (5 days) of recovery (e.g., Day 7 of FIG. 63)

FIG. 80A-B presents exemplary data of measured ethanol levels in the perfusion medium.
FIG. 80A: After twenty-four hours of perfusion.
FIG. 80B: After forty-eight hours of perfusion.

FIG. 81 Representative time-line of an ethanol or high fat dosing Liver-Chip study plan. Oxidative stress was measured by staining of ROS-positive hepatocytes with MitoSOX™ Red (a fluorogenic dye specifically targeted to mitochondria in live cells. Oxidation of MitoSOX™ Red reagent produces red fluorescence.

FIG. 82A-E Ethanol consumption is well known to modify gut permeability, increasing the levels of LPS in the blood. Here we demonstrate how the Liver-Chip can recapitulate similar changes.
FIG. 82A shows bright field micrographs of hepatocytes (upper row). Oxidation (red) is shown in the lower row stained with MitoSOX™ Red. Hepatocytes were treated, left to right, vehicle; 0.08%; 0.08%+LPS; and 0.16% ethanol.

The Liver-Chip alcohol-induced steatosis (FIG. 82B) reverted after ethanol-free recovery (FIG. 82C). LPS presence significantly increased oxidative stress (FIG. 82D) even after 5 days of recovery (FIG. 82E) demonstrating a role in alcoholic liver disease mechanisms of action.

FIG. 83 Liver-Chip demonstrated increase of cholesterol release (effluent) (top-left) and lysate (top-right and middle-left); and viability (middle-right) glucose (bottom-left) and glycogen (bottom-right) when incubated with ethanol at physiologically relevant blood alcohol concentration (BAC). Modifications in glucose release levels were observed with ethanol concentration above 0.32%.

Liver-Chip under ethanol dosing showed increased cholesterol and glycogen storage. Exemplary use of Albumin as a hepatocytes viability marker demonstrating that ethanol dosing condition were sublethal for the hepatocytes at the Liver-Chip.

### DETAILED DESCRIPTION OF THE INVENTION

The present invention is related to the field of liver disease. Solid substrates comprising microfluidic channels (e.g., microchips) are configured to support growing and differentiating hepatocytes and are contemplated to provide a suitable environment for the development of a fully functional liver tissue. These solid substrates can be used to induce various toxicity conditions in the liver tissue subsequent to the exposure to various chemicals. For example, chronic exposure to ethanol induces a clinical state of alcoholic liver disease in the liver tissue. Alternatively, certain disease states can result in the development of non-alcoholic liver diseases (e.g., non-alcoholic steatohepatitis; NASH).

Although it is not necessary to understand the mechanism of an invention, it is believed that liver disease is progressive in nature, transitioning from simple fatty tissue (steatosis) to fibrosis and/or cirrhosis. For example, steatosis may be induced by high fat diets (HFD), fructose, drugs (e.g., ethanol) and or viral infections. At this point, the liver toxicity stage is termed “non-alcoholic fatty liver disease” (NAFLD) where the primary symptom is steatosis. With continued exposure to such toxins the liver progresses into the stage termed “non-alcoholic steatohepatitis” (NASH) where the primary symptoms are steatosis and inflammation. The hepatic inflammation associated with metabolic disorder is believed to be strongly impacted by activate Kupffer cells. Eventually, the continued exposure of the liver to these inflammatory state results in tissue fibrosis and liver cirrhosis. At this point, the Kupffer cell-mediated inflammation and associated metabolism dysregulation has activated hepatic stellate cells that resulted in scar tissue formation. This compromised conditions predisposes the liver to further damage caused by release of cytokines and adipokines, oxidative stress and general mitochondrial dysfunctions. See, FIG. 14.

Liver metabolism plays a role in the metabolic degradation of alcohol and other drugs (e.g., prescription and recreational drugs). Further, the liver maintains the homeostatic balance of many endogenous compounds including, but not limited to, free fatty acids, ketone bodies, very low density lipoproteins (VLDLPs), bile acids, amino acids, proteins, albumin, glucose, lactate, urea and/or bilirubin. Altered liver metabolism can lead to disorders including, but not limited to, non-alcoholic fatty liver disease (NALFD), non-alcoholic steatohepatitis (NASH), choleostasis, fibrosis, cirrhosis and/or hepatocellular carcinoma. In regards to NAFLD and NASH, these two liver diseases have worldwide prevalence of 24% (9-36%) and 3-8%, respectively. In the United States, approximately 75-100 million individuals have symptoms of these two liver disorders at a combined annual cost of $103 billion ($1,613 per patient). The prevalence of both conditions have recently doubled where NAFLD increased from 15% in 2005 to 25% in 2010 and NASH increased from 33% to 59% during the same time period. NAFLD and NASH are the second most common indication for liver transplantation in the USA after chronic hepatitis C. There currently exists a problem in the art that no treatment or reliable biomarkers (e.g., non-invasive methods) available to identify and diagnose patients for treatment that can avoid the serious progression to liver cirrhosis and potential mortality.

Conventional human and physiological in vitro models for ASH contain limiting factors that prevent the development of clinically effective new treatments. Most current in vitro and in vivo models for ALD use a non-physiological exposure to ethanol (as it concerns both dose and duration), which is an important limiting factor for the translation of this data to humans. The present invention provides a human-relevant model for ALD, providing a microfluidic tissue testing system for evaluating human-relevant blood alcohol concentrations (BAC). Furthermore, the system uses human primary hepatocyte co-culture with human primary liver sinusoidal endothelial cells (LSECs) in the presence and absence of human primary Kupffer cells (KCs) and can evaluate different ethanol dosing regimens by varying tissue exposure parameters including, but not limited to, concentration, duration and/or frequency. The regimens can result in specificity of endpoints related to generally accepted alcohol consumption based categories including, but not limited to: (A) moderate, (B) binge and (C) heavy drinkers.

The microfluidic tissue testing system as disclosed herein is highly adaptable and compatible with well-established in vitro techniques. Alternatively, the microfluidic tissue testing systems can be applied to other tissues, other diseases and generalized drug toxicity modeling, because of the human-relevancy to the in vitro development of tissue intracellular architecture that mimics the natural state.

### I. Alcohol-Induced Tissue Injury

Both animal and human studies provide evidence on alcohol-induced liver injury and dysfunction, and it is well validated that ASH may be reversed after 4-6 weeks of alcohol abstinence suggestive of the temporary cell damage in the early phases of the disease. Despite the number of experimental studies, there is no yet approved therapy for any stage of ALD, emphasizing the need for research for novel therapeutic interventions and better understanding of the self repair mechanisms operating in the early stages of the disease. Rehm et al., “Global burden of disease and injury and economic cost attributable to alcohol use and alcohol-use disorders” Lancet 2009; 373:2223-2233: Stickel et al., “Pathophysiology and Management of Alcoholic Liver Disease: Update 2016” Gut and Liver 2017; 11:173-188; and Magdaleno et al., “Key Events Participating in the Pathogenesis of Alcoholic Liver Disease” Biomolecules 2017; 7:9.

In spite of being a cause of morbidity and mortality in the world, there are currently no effective strategies that can prevent or treat alcoholic liver disease (ALD) due to a lack of human and physiological-relevant research models. The microfluidic tissue systems as contemplated herein provide a human microphysiological system that recapitulates tissue architecture to achieve organ level physiological functions. The data shown herein demonstrates the development of a mature, functional human liver-on-chip that has been maintained for around three weeks in culture.

Alcohol consumption accounts for approximately 3.8% of all global deaths and 4.6% of global disability-adjusted life-years. Alcohol use disorders (AUD) are the most frequent cause of liver cirrhosis in Europe, and ALD the most important cause of death due to alcohol in adults worldwide and in the United States. Despite the profound economic and health impact, there is no Food and Drug Administration-approved therapy for any stage of ALD, emphasizing the need for research into therapeutic interventions during the early initiating stages of the disease.

A. ALD Spectrum and Pathophysiology

ALD is believed to include a spectrum of liver diseases including, but not limited to, fatty liver, alcoholic steatohepatitis (ASH), fibrosis and liver cirrhosis. Liver cirrhosis accounts for 16.6% of mortality worldwide, and the most common inducing factor is alcohol-induced damage on liver. ASH usually develops in approximately 90% of individuals who drink more than 60 g alcohol/day, regularly. Beyond fatty liver, ALD comprises a continuum of partly overlapping liver abnormalities with variable degrees of inflammation and progressive fibrosis in 10% to 35% of alcoholics, and liver cirrhosis in approximately 10% to 15% of heavy drinkers. A great concern is the rising incidence of hepatocellular carcinoma (HCC) which evolves in approximately 1% to 2% of alcoholic cirrhotics per year.

ALD is believed to be completely reversible after 4-6 weeks of abstinence, even if fibrosis has already developed. Chronic alcohol consumption increases gut permeability, permitting the translocation of LPS from the intestinal lumen to the portal circulation. Alcohol consumption is a known cause of increased gut permeability, facilitating the translocation of gut microbiota into the circulation. In turn, this leads to increased liver exposure to LPS, which causes liver injury via TLR4 activation. A wide range of cytokines were assessed in samples of ALD patients. These include tumor necrosis factor (TNF)-alpha, various interleukins (IL) such as IL-1beta, IL-4, IL-6, IL-10, IL-12, IL-18 and interferon (IFN)-gamma. Lee et al., “3D alcoholic liver disease model on a chip” Integr. Biol. (2016); Toxicological Sciences 132(1):131-141 (2013); and “Alcoholic liver disease: The gut microbiome and liver crosstalk” Alcohol Clin Exp Res. 39(5):763-775 (2015).

In one embodiment, the present invention contemplates a method for developing alcoholic liver disease in liver cells on a microchip (e.g., a hepatocyte microchip). In one embodiment, alcoholic liver disease is developed in the presence of 0.5 to 4 μl/ml ethanol. In one embodiment, the liver cells develop reversible alcoholic liver disease. In one embodiment, the liver cells develop reversible alcoholic liver disease. In one embodiment, the hepatocyte microchip recapitulates ALD in a human and/or in vitro by modeling: i) steatosis progression; ii) steatosis reversibility;

B. ALD Mechanisms

Histological hallmarks of ALD may include, but are not limited to, steatosis, inflammation and fibrosis and are believed to be a result of interrelated and consecutive pathophysiological events in the context of continuous alcohol exposure. For example, alcoholic fatty liver may be an initial liver lesion in alcoholics and could be a result of biochemical disruptions including, but not limited to, disrupted lipid turnover, decreased fatty acid oxidation, increased lipogenesis (e.g., fatty acid and triglyceride synthesis) by dysregulation of steatogenic enzymes and/or transcription factors. Whether, and how, alcohol consumption affects enzymatic function, however, is still unclear.

A pivotal component in the evolution of ALD is the direct toxicity of the first metabolite of alcohol degradation, acetaldehyde (AA). Two major enzyme systems can metabolize alcohol to AA via oxidative degradation, of which alcohol-dehydrogenase (ADH) is the system primarily responsible for the processing of lower amounts of alcohol. ADH is generally located in the cytosol and cannot be upregulated upon demand (i.e., not inducible). In contrast, cytochrome P450 2E1 (CYP2E1) located in microsomes is inducible and can be upregulated 10- to 20-fold in heavy drinkers. Both enzyme systems generate AA, a highly reactive toxic and mutagenic metabolite, by which they not only degrade ethanol (and other organic substances), but also contribute to alcohol-related toxicity. Apart from generating AA, CYP2E1 also contributes to oxidative damage by the formation of reactive oxygen species (ROS) such as superoxide anion and hydrogen peroxide.

C. Liver Macrophages (Kupffer Cells) and ASH

It has been generally accepted that the pathogenesis of ALD is multifactorial in which liver parenchymal cells (e.g., hepatocytes) and liver non-parenchymal cells are involved. Accumulating evidence has demonstrated that Kupffer Cells (KCs) may play a role in the pathogenesis of both chronic ALD and acute ALD. Zeng et al., “Critical Roles of Kupffer Cells in the Pathogenesis of Alcoholic Liver Disease: From Basic Science to Clinical Trials” Frontiers in Immunology. 2016; 29(7):538. KCs also may play a role in host defense by removing foreign, toxic and/or infective substances from the circulatory system and have been demonstrated to be involved in the pathogenesis of many kinds of liver diseases. Inflammation can occur as a signature feature in ASH, and as a major driving force for fibrogenesis leading to fibrosis and/or cirrhosis.

1. Kupffer Cells and Cytokine Release.

The data presented herein determined effect of Kupffer : Hepatocyte ratio by measuring the cytokine response to LPS stimulation on hepatocyte microchips cultured either with or without Kupffer cells. TNF-α release was measured after treatment with LPS (lug/ml of LPS for 24 hours). The data show that TNF-α release is positively correlated in proportion to the Kupffer-hepatocyte cell ratio. See, FIG. 43.

2. Beneficial Features of Kupffer Cells and Stellate Cells.

Kupffer cells and Stellate Cells have beneficial effects on hepatocytes.

CYP1A1 Enzyme Activity was measured in Rat Liver-on-Chip (pmol/min/million cells). Three embodiments of rat Liver-on-Chip appear to have significantly higher levels of CYP1A1 activity than conventional plate culture. Three types of rat Liver-on-Chip show in vivo-relevant CYP1A1 activity over long-term culture; Co-culture (Liver Chip); Tri-culture (Liver Chip); Quadruple-culture (Liver Chip).

D. Fibrosis

Fibrosis can present a significant challenge for cell function and survival. For example, fibrotic environments are known to induce cell de-differentiation, migration, proliferation, and promote organ failure. Fibrosis can also be a challenge for drug discovery. Fibrosis is an over-response to organ injury that results in an alteration of the cellular architecture, such as in the liver. See, FIG. 41A-B. Elimination of the injury's cause is not enough in a fibrotic environment such that a reversion of the fibrotic state is the recommended treatment. Various compounds and/or drugs may have different efficacy in a fibrotic environment, because the fibrotic tissue changes conditions including, but not limited to, drug delivery efficiency and/or cellular dose-response kinetics. In one embodiment, the present invention contemplates a method for inducing fibrosis in a hepatocyte microchip culture. In one embodiment, the fibrosis is mediated by the stimulation of co-cultured stellate cells by TNF-α. See, FIGS. 46A-B.

Chronic liver disease may result in lesions such as fibrosis that, in essence, resemble a process of excessive wound healing characterized by an increased fibrogenesis and a decreased fibrolysis. For example, in progressive fibrosis, liver parenchyma may be replaced by an extracellular matrix produced by activated hepatic stellate cells (HSC), resulting in a distorted liver architecture and progressive functional impairment. Various triggers can activate Kupffer cells and other inflammatory cells, which may lead to the production of profibrogenic cytokines platelet-derived growth factor and/or transforming growth factor-β1, which can stimulate HSCs to produce molecules including, but not limited to, collagens, noncollagenous glycoproteins, proteoglycans and/or glycosaminoglycans in concentrations up to approximately 10-fold as compared to normal liver tissue. Here, fibril-forming collagens type I and III make up for >80% of total liver collagen. In turn, matrix-degrading enzymes termed matrix-metalloproteinases (MMP) are downregulated by their corresponding tissue inhibitors (TIMP). In ALD, HSCs can be stimulated by AA, ROS, leptin and/or lipid peroxides.

E. Experimental ALD Models

In spite of being one cause of morbidity and mortality in the world, currently, there are no effective strategies that can prevent or treat alcoholic liver disease (ALD), due to a lack of human and physiologically-relevant research models. Studying ALD experimentally has been extremely difficult since no animal model exists that closely mirrors all relevant features of severe ALD in humans. Rodents are notoriously resistant to the hepatotoxic effects of alcohol, and rats and mice only develop significant chronic liver injury when exposed to alcohol in combination with a second toxin or major dietary manipulations (e.g., choline/methionine deficiency) and still do not produce a histological picture that fully models human ALD. A number of studies have shown that viability in 2D models is significantly decreased by ethanol concentrations greater than 100 mM (i.e., ˜0.4%), however, those values are not physiological relevant. It is generally accepted that physiologically relevant concentrations of ethanol generally range between approximately 5-20 mM.

Currently, there are few in vitro models that are able to sustain, at least in part, the fibrotic stage of human liver disease and there is no current literature report of a liver fibrosis in vitro model for ALD. Karim et al., “An in vitro model of human acute ethanol exposure that incorporates CXCR3- and CXCR4-dependent recruitment of immune cells” Toxicological Sciences 132(1):131-141 (2013). Embodiments of the present invention are the result of the development and characterization of a microfluidic tissue testing system providing an alcoholic steatosis model. In one embodiment, the testing system comprises a three dimensional HSC hepatocyte microchip that can support the fibrotic stage of the disease. Lee et al., “3D alcoholic liver disease model on a chip” Integrative Biology 2016, 14:8(3):302-308

Although it is not necessary to understand that mechanism of an invention, it is believed that HSCs are quiescent vitamin A-storing pericytes which are located in the perisinusoidal space between the LSECs and hepatocytes. HSCs represent about 5%-8% of cells in a normal liver. Under normal conditions HSCs store up to 80% of the total body vitamin A in cytoplasmic lipid droplets. Higashi et al., “Vitamin A storage in hepatic stellate cells in the regenerating rat liver: with special reference to zonal heterogeneity” Anat Rec A Discov Mol Cell Evol Biol 286:899-907 (2005). Activation of quiescent vitamin A-storing HSCs into a vitamin A-depleted myofibroblast-like cell type plays a role in the cellular process of hepatic fibrogenesis. Activation of HSCs into a myofibroblast-like phenotype (e.g., transdifferentiation) is characterized by observations including, but not limited to, an increase of microfilament protein A-smooth muscle actin (a-SMA), cell proliferation, cell migration, production of ECM (collagen deposition) and tissue remodeling by producing cytokines and growth factors. HSCs cultured on plastic dishes in the presence of fetal calf serum (FCS) immediately attach, start to proliferate and undergo spontaneous transdifferentiation (activation) into a myofibroplastic phenotype, very similar to the process observed in chronic liver diseases. Wirz et al., “Hepatic stellate cells display a functional vascular smooth muscle cell phenotype in a three-dimensional co-culture model with endothelial cells” Differentiation 76:784-794 (2008). On one hand, HSC characteristics promoted a good understanding of HSC activated phenotypes, but on the other hand also made difficult to study HSC quiescent stages. In one embodiment, the presently disclosed microfluidic tissue testing system is configured to modify the microenvironment of an in vitro tissue culture to alternate between HSC activation and HSC quiescent by alteration of the fluid flow characteristics and components therein. For example, these assessments can be made by: (1) identifying the presence/absence of fibrotic markers; (2) determining the presence of reversibility points for the ASH/fibrotic pathway; (3) determining LPS and ethanol dosing concentration/exposure time in order to the recapitulate main progressive ALD stages.

F. Metabolic Biomarkers

The data herein shows several biomarkers that are useful to identify several developmental stages of ALD or NALD. For example, liver accumulation of fatty deposits were observed in developmental stages of all three conditions. See, FIG. 37A-B. Biomarkers such as polyploidy/cell proliferation and glucose levels can be used in these three conditions. See, FIG. 41. Biomarkers such a cholesterol, triglycerides and/or reactive oxygen species (ROS) can also be used in these three conditions. See, FIGS. 37C-D.

### II. Non-Alcoholic Liver Disease (NALD & NASH)

In one embodiment, the present invention contemplates a variety of methods to: i) develop NAFLD cell culture models; ii) strategies to identify changes in cellular architecture resulting from the expression of NAFLDs; and iii) screening regimens to identity potential therapeutic candidates to treat NAFLD. Non-alcoholic fatty liver disease (NAFLD) or non-alcoholic steatohepatitis (NASH) is one of the types of fatty liver which occurs when fat is deposited (steatosis) in the liver due to causes other than excessive alcohol use. Most commonly, symptoms of NASH include, but are not limited to, mild jaundice, inflammation, liver cell damage and/or steatohepatitis (e.g., fatty liver).

The percentage of people with NAFLD ranges from 9 to 36.9% in different parts of the world. Omagari et al., (2002) “Fatty liver in non-alcoholic non overweight Japanese adults: incidence and clinical characteristics” J Gastroenterol Hepatol: 1098-1105; Hilden et al., (1977) “Liver histology in a ‘normal’ population—examinations of 503 consecutive fatal traffic casualties” Scand J Gastroenterol. 12(5); and Shen et al., (2003). “Prevalence of nonalcoholic fatty liver among administrative officers in Shanghai: an epidemiological survey”. World J Gastroenterol. 9:1106-10. Approximately 20% of the United States population have non-alcoholic fatty liver, and the number of people affected is increasing. This means about 75 to 100 million people in the United States are affected. Lazo et al., (2011) “Non-alcoholic fatty liver disease and mortality among US adults: prospective cohort study”. BMJ 343 (November 18); and Rinella, ME (2015). “Nonalcoholic fatty liver disease: a systematic review” JAMA 313(22):2263-73. The rates of NAFLD is higher in Hispanics, which can be attributed to high rates of obesity and type 2 diabetes in Hispanic populations. Flegal et al., (2002). “Prevalence and trends in obesity among US adults, 1999-2000” JAMA 288(14):1723-7.

Because obesity is becoming an increasingly common problem worldwide, the prevalence of NAFLD has been increasing concurrently. Moreover, boys are more likely to be diagnosed with NAFLD than girls with a ratio of 2:1. Barshop et al., (2008) “Review article: epidemiology, pathogenesis and potential treatments of pediatric non-alcoholic fatty liver disease”. Aliment Pharmacol Ther. 28(1):13-24; Baldridge et al., (1995) “Idiopathic steatohepatitis in childhood: a multicenter retrospective study” Journal of Pediatrics 127(5):700-704; and Kinugasa et al., (1984) “Fatty liver and its fibrous changes found in simple obesity of children” Journal of Pediatric Gastroenterology Nutrition. 3(12):408-414. Non-alcoholic fatty liver disease is also more common among men than women in all age groups until age 60, where the prevalence between sex equalize. This is due to the protective nature of estrogen. Lobanova et al., (2009). “NF-kappaB suppression provokes the sensitization of hormone-resistant breast cancer cells to estrogen apoptosis” Mol Cell Biochem. 324.

Fatty liver and NASH can occur at all ages, with the highest rates in the 40- to 49-year-old age group. It is the most common liver abnormality in children ages 2 to 19. Pediatric nonalcoholic fatty liver disease (NAFLD) was first reported in 1983. It is currently the primary form of liver disease among children. Pacifico et al., (2010). “Pediatric nonalcoholic fatty liver disease: a clinical and laboratory challenge” World J Hepatol (2 ed.). 7: 275-288; Moran et al., (1983) “Steatohepatitis in obese children: a cause of chronic liver dysfunction” American Journal of Gastroenterology. 78 (6): 374-7; and Papandreou et al., (2007) “Update on non-alcoholic fatty liver disease in children” Clinical Nutrition. 16:409-415.

NAFLD is the most common liver disorder in developed countries. Shaker et al. (2014) “Liver transplantation for nonalcoholic fatty liver disease: New challenges and new opportunities”. World Journal of Gastroenterology: WJG. 20 (18): 5320; and Rinella ME (2015). “Nonalcoholic fatty liver disease: a systematic review” JAMA 313(22): 2263-73. Usually, NAFLD and NASH cause few or no noticeable symptoms in a patient. About 12 to 25% of people in the United States has NAFLD while NASH affects between 2 to 5% of people in the United States. Nonetheless, it is known that some medical conditions including, but not limited to, obesity, metabolic syndromes and/or type 2 diabetes increase the likelihood that NAFLD and/or NASH may develop. NAFLD is related to insulin resistance and the metabolic syndrome and may respond to treatments originally developed for other insulin-resistant states (e.g. diabetes mellitus type 2) such as weight loss, metformin, and thiazolidinediones. NAFLD can also be caused by some medications including, but not limited to, amiodarone, antiviral drugs (nucleoside analogues), aspirin, corticosteroids, methotrexate, tamoxifen and/or tetracycline. Adams et al., (2006) “Treatment of non-alcoholic fatty liver disease”. Postgrad Med J. 82 (967): 315-22. Up to 80% of obese people have the disease. Sanyal, A J (2002). “AGA Technical Review on Nonalcoholic Fatty Liver Disease.”. Bethesda, Md.: American Gastroenterological Association. Soft drinks have been linked to NAFLD due to high concentrations of fructose, which may be present either in high-fructose corn syrup or, in similar quantities, as a metabolite of sucrose. The quantity of fructose delivered by soft drinks may cause increased deposition of fat in the abdomen. Nseir et al., (2010). “Soft drinks consumption and nonalcoholic fatty liver disease”. World Journal of Gastroenterology. 16 (21): 2579-2588; and Allocca et al., (2010). “Emerging nutritional treatments for nonalcoholic fatty liver disease”. In: Preedy V R; Lakshman R; Rajaskanthan R S. Nutrition, diet therapy, and the liver. CRC Press. pp. 131-146

NASH is regarded as a major cause of cirrhosis of the liver of unknown cause. Most people have a good outcome if the condition is caught in its early stages. NAFLD and/or NASH may be diagnosed based upon consideration of medical history in combination with a physical examination that may includes tests such as liver function blood tests, hepatic imaging tests and/or a liver biopsy. NAFLD may be associated with insulin resistance and metabolic syndrome (obesity, combined hyperlipidemia, diabetes mellitus (type II), and high blood pressure). Clark et al., (2003). “Nonalcoholic fatty liver disease: an under recognized cause of cryptogenic cirrhosis” JAMA 289 (22): 3000-4.

NAFLD/NASH have been associated with metabolic syndrome, a condition developed by a cluster of risk factors that contribute to the development of cardiovascular disease and type 2 diabetes mellitus. Studies have demonstrated that obesity and the corresponding development of insulin-resistance in particular are thought to be key contributors to the development of NAFLD. Cortez-Pinto H et al., (1999) “Nonalcoholic fatty liver: another feature of the metabolic syndrome?” Clinical Nutrition. 18 (6): 353-8; Marchesini et al., (2001) “Nonalcoholic fatty liver disease: a feature of the metabolic syndrome” Diabetes 50 (8); Nobili et al., (2006) “NAFLD in children: A prospective clinical-pathological study and effect of lifestyle advice”. Hepatology. 44 (2): 458-465; Pagano et al,. (2002) “Nonalcoholic steatohepatitis, insulin resistance, and metabolic syndrome: further evidence for an etiologic association”. Hepatology. 35: 367-372; and Schwimmer et al., (2008) “Cardiovascular risk factors and the metabolic syndrome in pediatric nonalcoholic fatty liver disease” Circulation. 118 (12): 277-283.

Genetic bases for NAFLD and/or NASH have also been suggested. Polymorphisms (genetic variations) in the single-nucleotide polymorphisms (SNPs) T455C and C482T in APOC3 may be associated with fatty liver disease, insulin resistance, and possibly hypertriglyceridemia. Carriers of T-455C, C-482T, or both (not additive) had a 30% increase in fasting plasma apolipoprotein C3, 60% increase in fasting plasma triglyceride and retinal fatty acid ester, and 46% reduction in plasma triglyceride clearance. Although it is not necessary to understand the mechanism of an invention, it is believed that there is an association of metabolic disorders with feeding/fasting cycle dysregulation. In one embodiment, the present invention contemplates a hepatocyte microchip microfluidics system that supports a physiologically relevant modeling of fasting and feeding cycles that is relevant to human clinical applications.

It is further beloved that the prevalence of non-alcoholic fatty liver disease was 38% in NAFLD SNP carriers as compared to 0% in normal individuals. Subjects with fatty liver disease had marked insulin resistance. Petersen et al., (2010). “Apolipoprotein C3 Gene Variants in Nonalcoholic Fatty Liver Disease”. N. Engl. J. Med. 362 (12): 1082-9. Other genetic causes may be a congenital syndrome, identified by a family history of liver disease, or abnormalities in other organs, and those that present with moderate to advanced fibrosis or cirrhosis. Cassiman et al., (2008). “NASH may be trash”. Gut 57 (2):141-4.

NAFLD/NASH are considered to cover a spectrum of disease activity. This spectrum begins as fatty accumulation in the liver (hepatic steatosis). A liver can remain fatty without disturbing liver function, but by varying mechanisms and possible second insults to the liver may also progress to become non-alcoholic steatohepatitis (NASH), a state in which steatosis is combined with inflammation and fibrosis (steatohepatitis). NASH is a progressive disease: over a 10-year period, up to 20% of patients with NASH will develop cirrhosis of the liver, and 10% will suffer death related to liver disease. McCulough, Arthur J (August 2004). “The clinical features, diagnosis and natural history of nonalcoholic fatty liver disease”. Clinics in Liver Disease. 8 (3): 521-33. The exact cause of NAFLD is still unknown. However, both obesity and insulin resistance probably play a strong role in the disease process. The exact reasons and mechanisms by which the disease progresses from one stage to the next are not known.

Diagnostics of NAFLD/NASH generally finding elevated liver enzymes and a liver ultrasound showing steatosis. An ultrasound may also be used to exclude gallstone problems (cholelithiasis). A liver biopsy (tissue examination) is the only test widely accepted as definitively distinguishing NASH from other forms of liver disease and can be used to assess the severity of the inflammation and resultant fibrosis. Several non-invasive diagnostic tests have been developed that estimate: i) liver fibrosis (Halfon et al., (2008) “FibroTest-ActiTest as a non-invasive marker of liver fibrosis” Gastroenterol Clin Biol. 32 (6): 22-39; and ii) steatosis (Ratziu et al. (2006). “Diagnostic value of biochemical markers (FibroTest-FibroSURE) for the prediction of liver fibrosis in patients with non-alcoholic fatty liver disease” BMC Gastroenterology. 6: 6. Apoptosis has also been indicated as a potential mechanism of hepatocyte injury as caspase-cleaved cytokeratin 18 (M30-Apoptosense ELISA) in serum/plasma is often elevated in patients with NASH and tests based on these parameters have been developed. Sowa et al., (2013). “Novel algorithm for non-invasive assessment of fibrosis in NAFLD.” PLOS ONE. 8(4): e62439. Other diagnostic blood tests include, but are not limited to, erythrocyte sedimentation rate, glucose, albumin, and/or kidney function.

Currently, treatment of NAFLD and/or NASH is not treated with any FDA-approved mediations such that the clinical recommendation is usually weight loss. Ratziu et al., (2015). “Current efforts and trends in the treatment of NASH.”. Journal of Hepatology. 62 (1 Suppl): S65-75. Weight loss has been observed to reduce fatty liver, hepatic inflammation and hepatic fibrosis. No pharmacological treatment has received approval as of 2015. Some studies suggest diet, exercise, and antiglycemic drugs may alter the course of the disease. General recommendations include improving metabolic risk factors and reducing alcohol intake. While many treatments appear to improve biochemical markers such as alanine transaminase levels, most have not been shown to reverse histological abnormalities or reduce clinical endpoints Treatment of NAFLD may also include counseling to improve nutrition and consequently body weight and composition. Diet changes have shown significant histological improvement. Huang et al., (2005). “One-year intense nutritional counseling results in histological improvement in patients with non-alcoholic steatohepatitis: a pilot study”. Am. J. Gastroenterol. 100 (5): 1072-81. Specifically, avoiding food containing high-fructose corn syrup and trans-fats has been recommended. A systematic review and meta-analysis found that omega-3 fatty acid supplementation in those with NAFLD/NASH using doses approaching or higher than 1 gram daily (median dose 4 grams/day with median duration 6 months treatment) has been associated with improvements in liver fat. The best dose of omega-3 fatty acids for individuals with NAFLD/NASH is unclear. Epidemiological data have suggested that coffee consumption may be associated with a decreased incidence of NAFLD and may reduce the risk of liver fibrosis in those who already have NAFLD/NASH. Olive oil consumption, as part of the Mediterranean diet, is also a reasonable dietary intervention; the optimal dose of olive oil supplementation for people with NAFLD/NASH has not been well-established. Few studies have been performed to evaluate the respective impact of a diet rich in avocados, red wine, tree nuts, or tea in people with NAFLD/NASH. However, limited evidence suggests that avocados may improve other areas of cardiovascular health (i.e., lipid profile) and their addition to a balanced diet is reasonable.

A. High Fat Diet Liver Injury

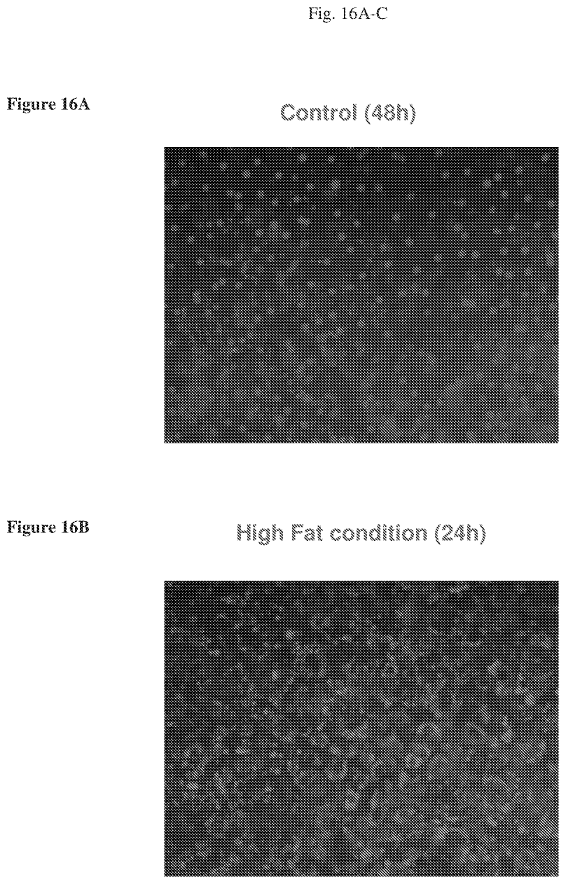

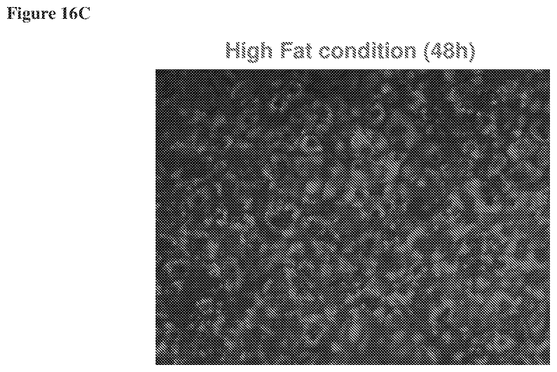

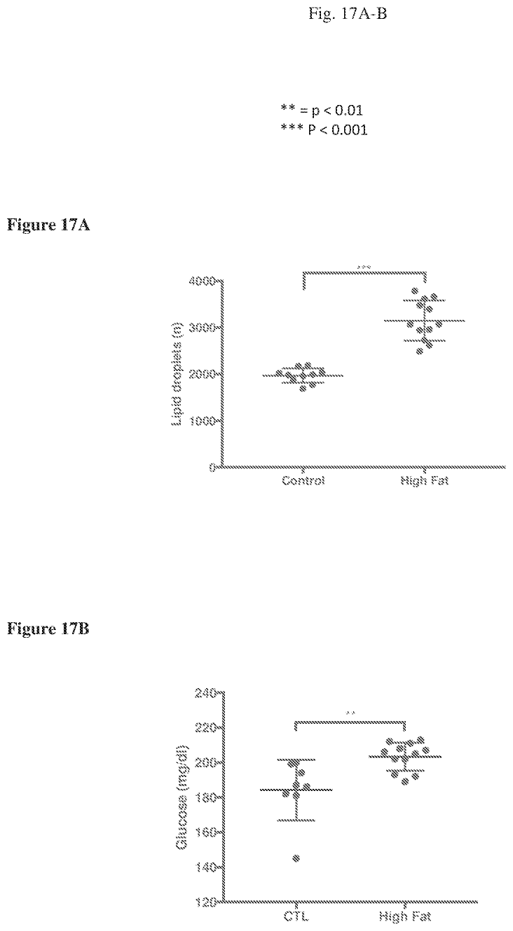

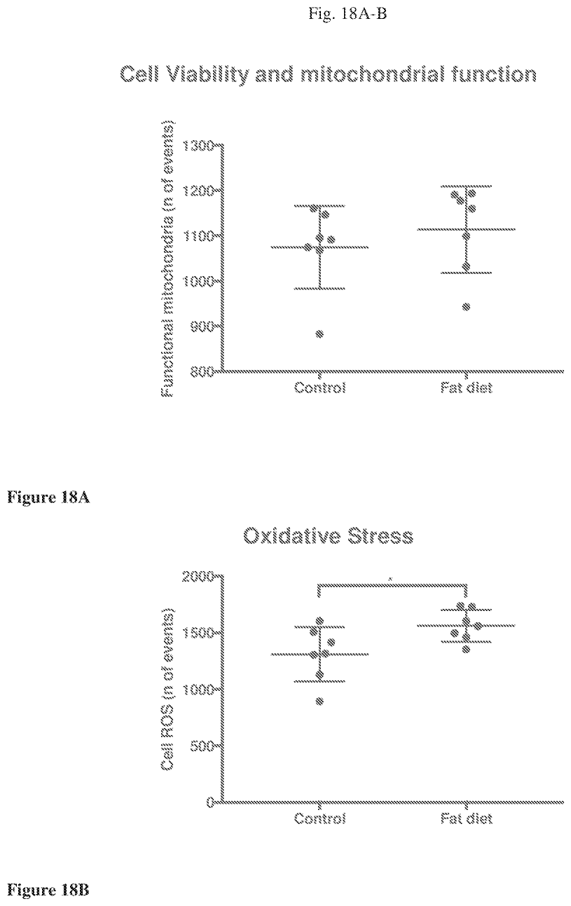

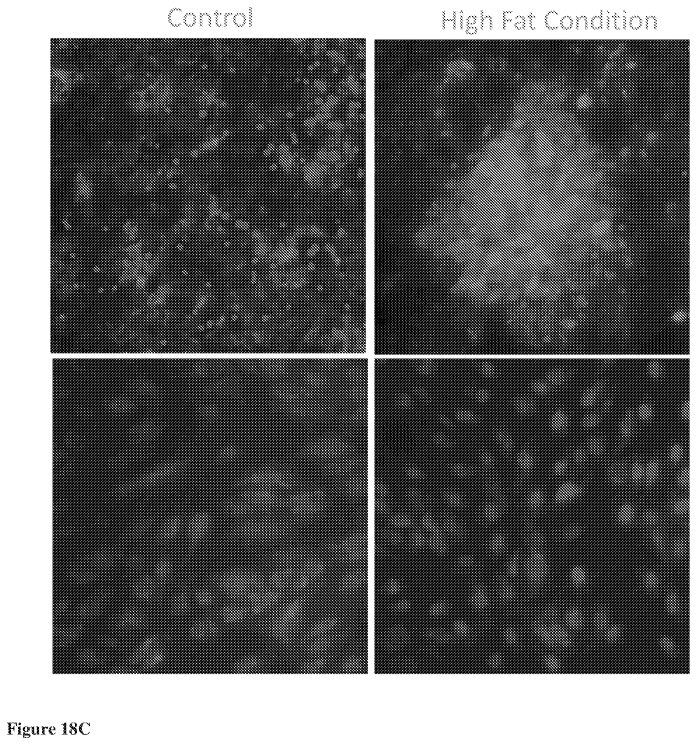

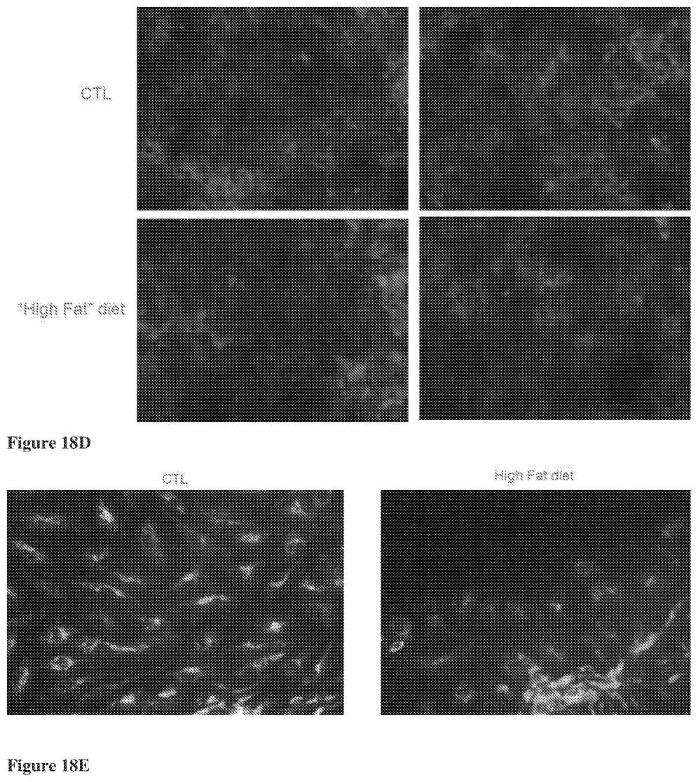

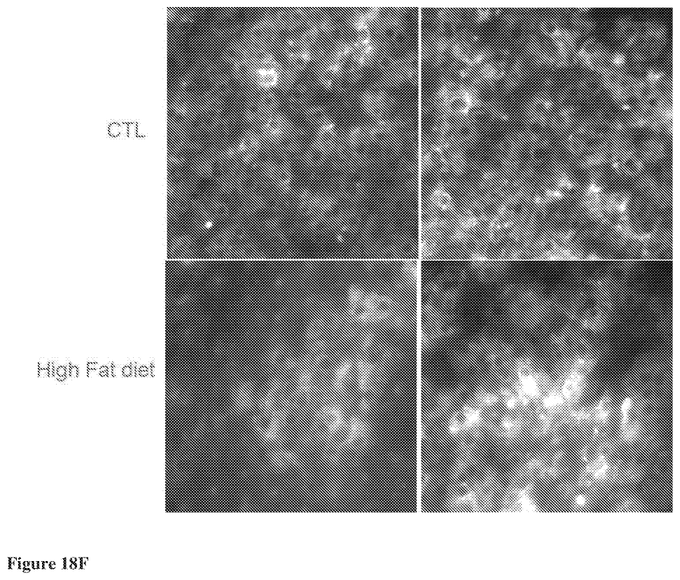

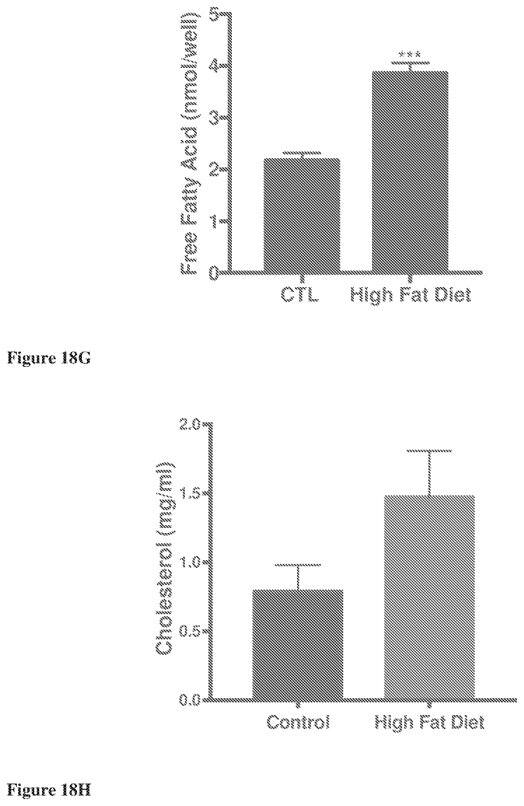

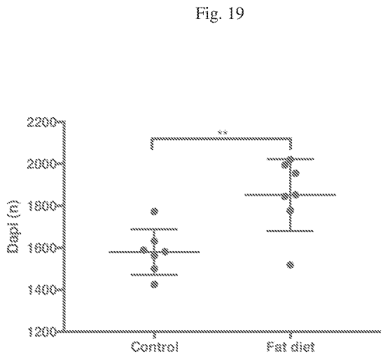

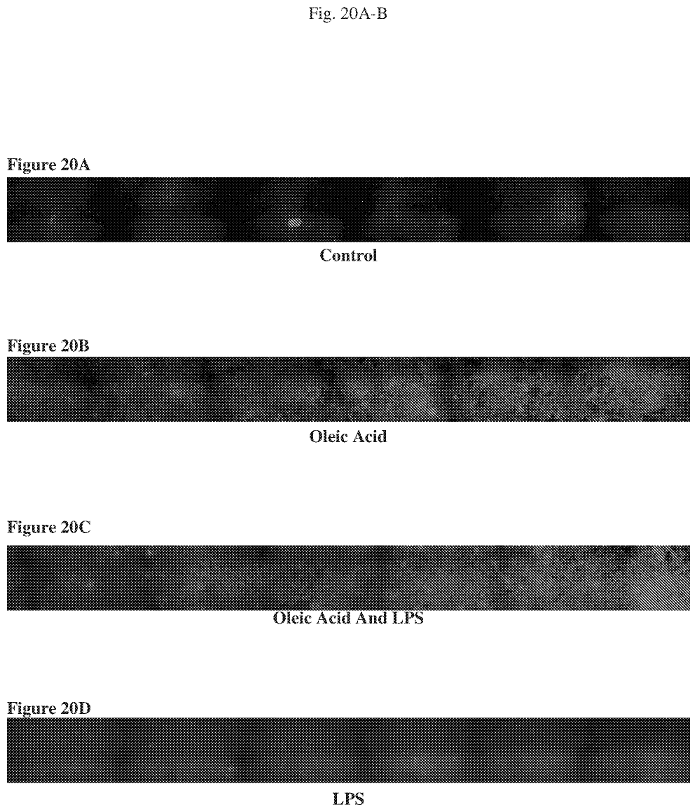

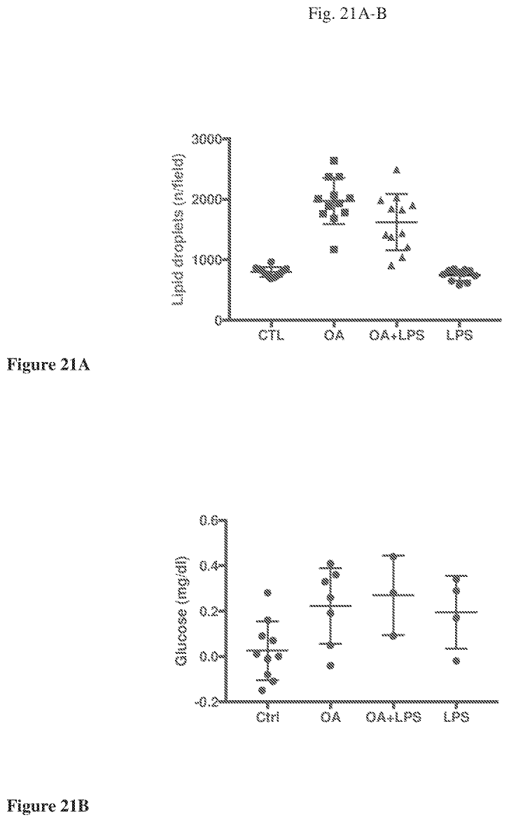

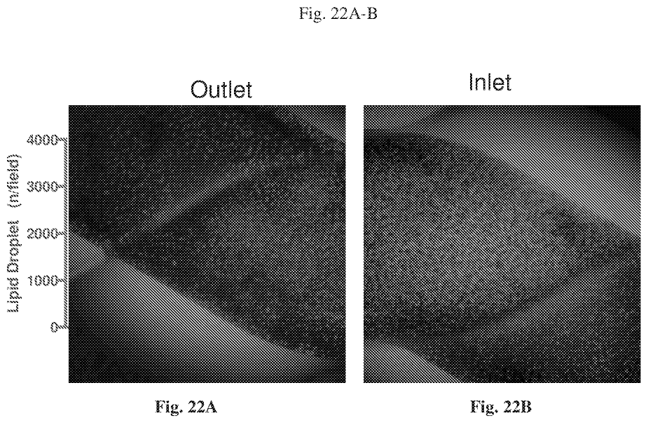

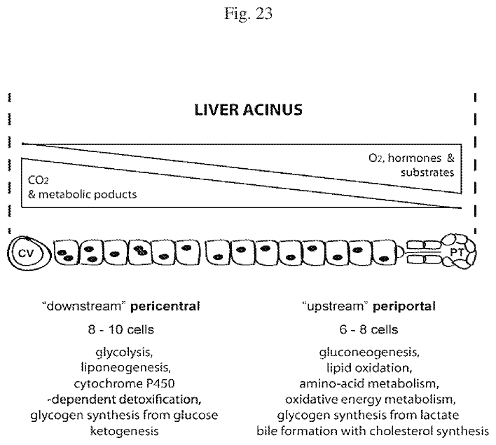

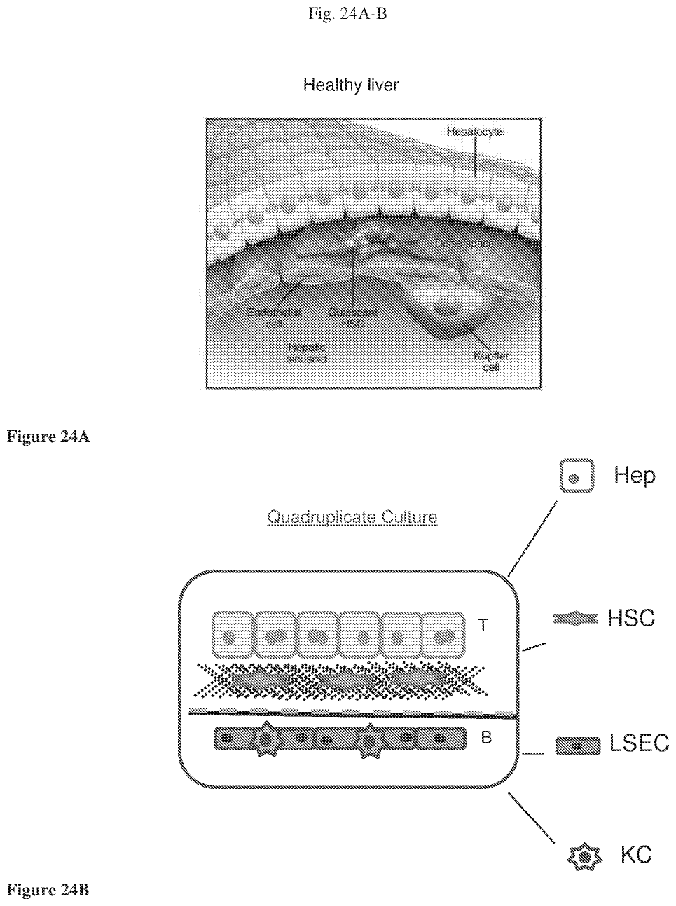

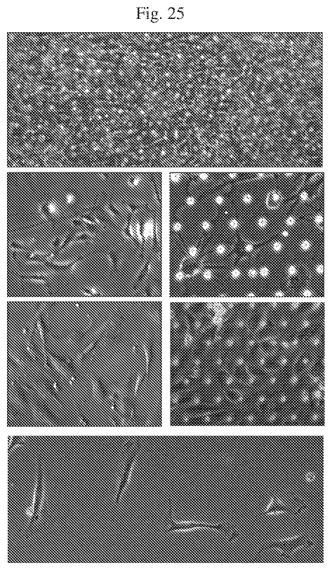

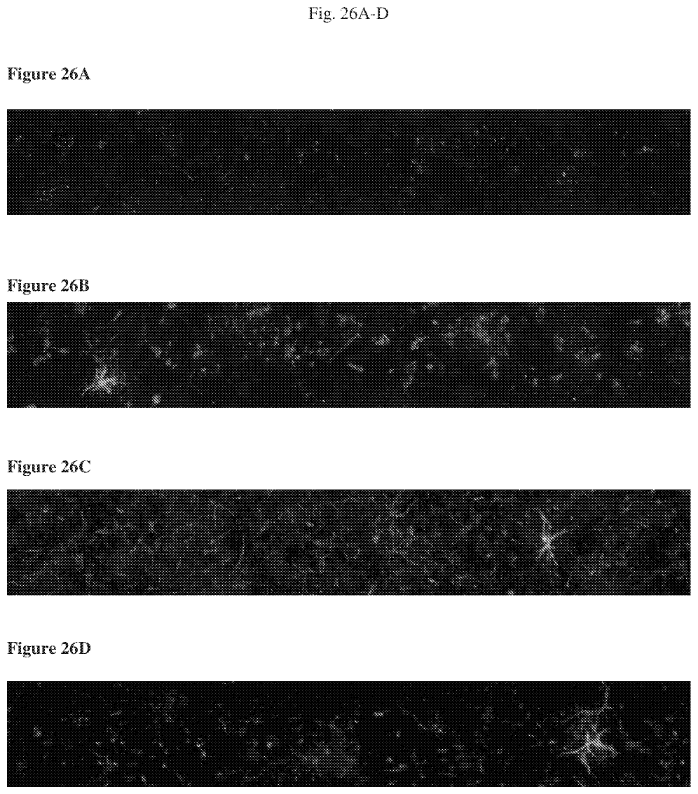

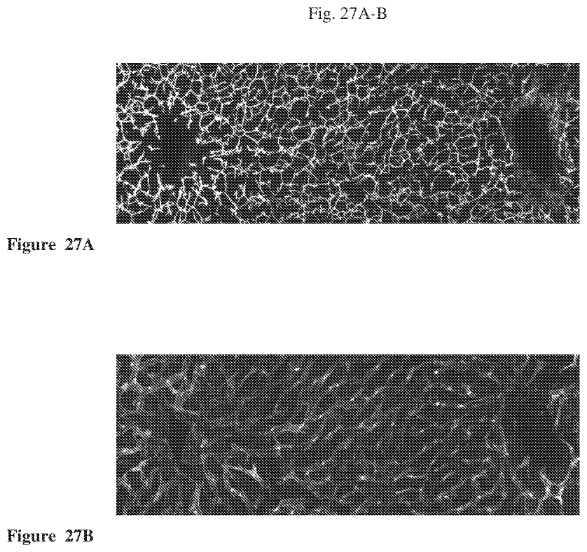

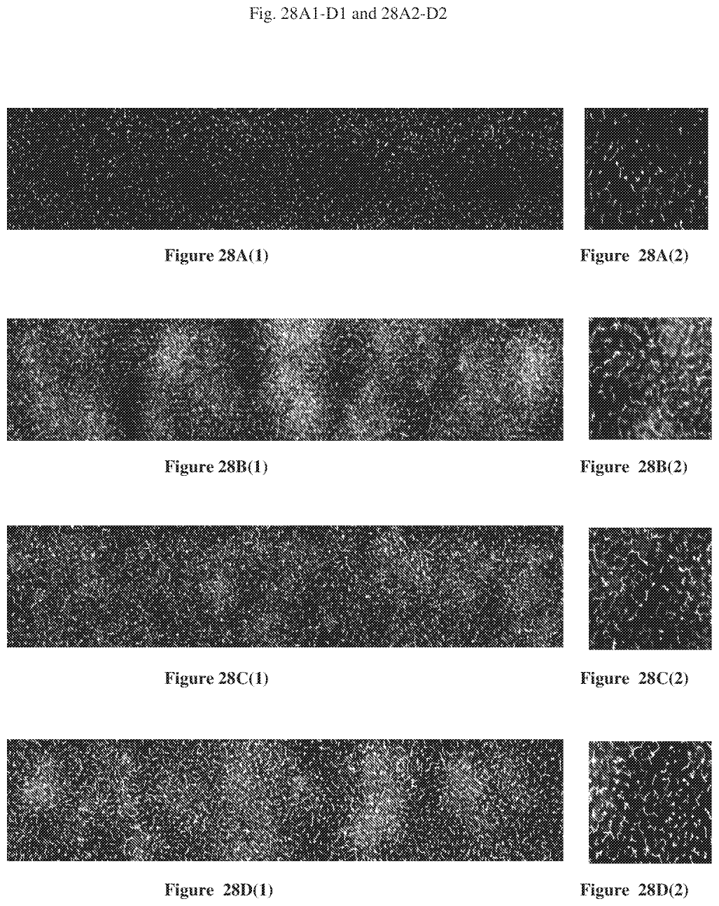

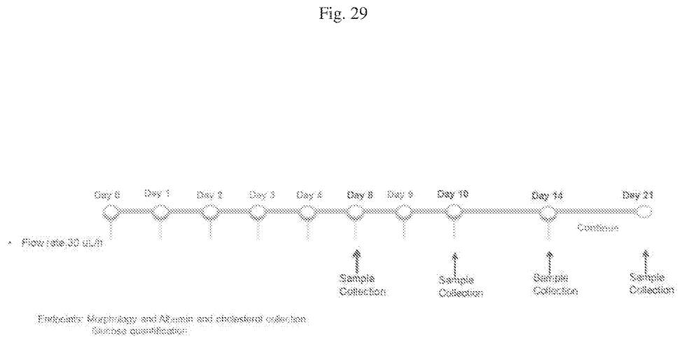

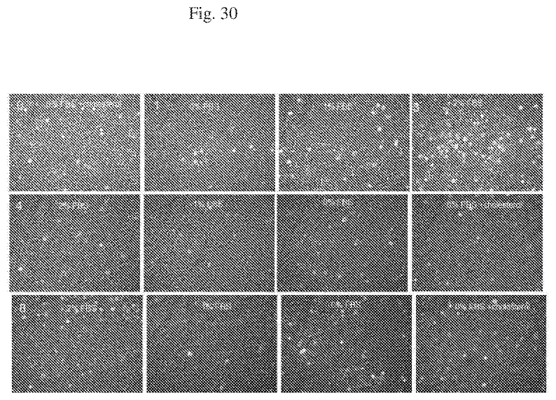

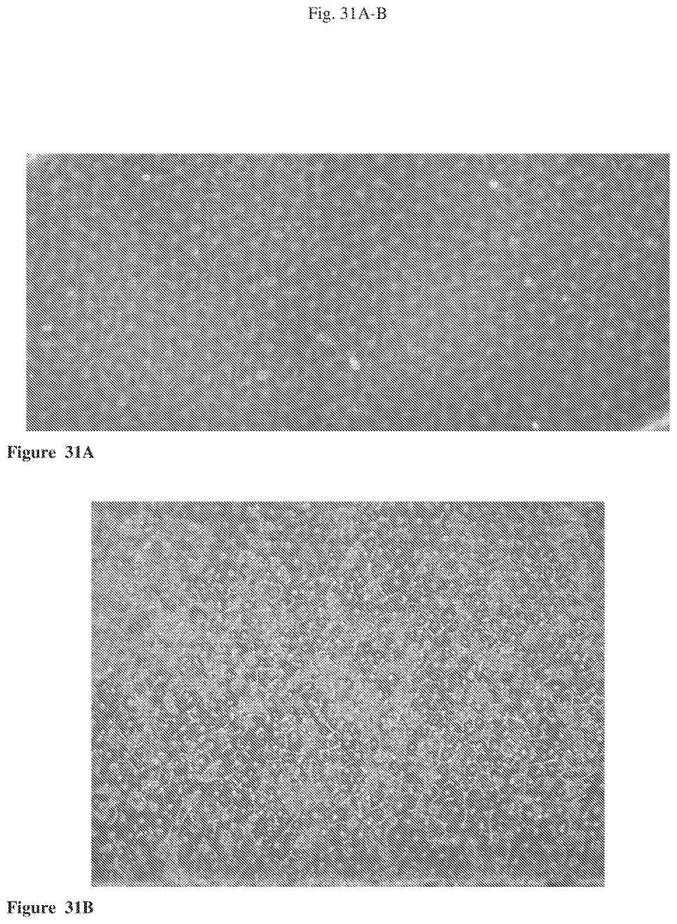

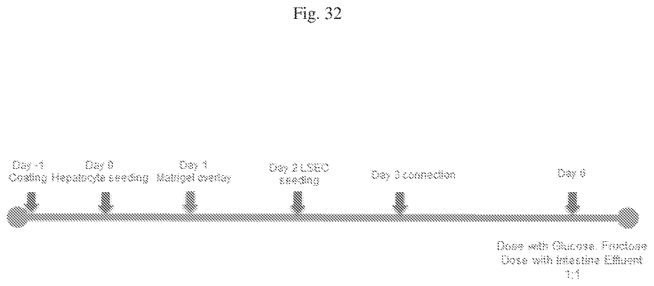

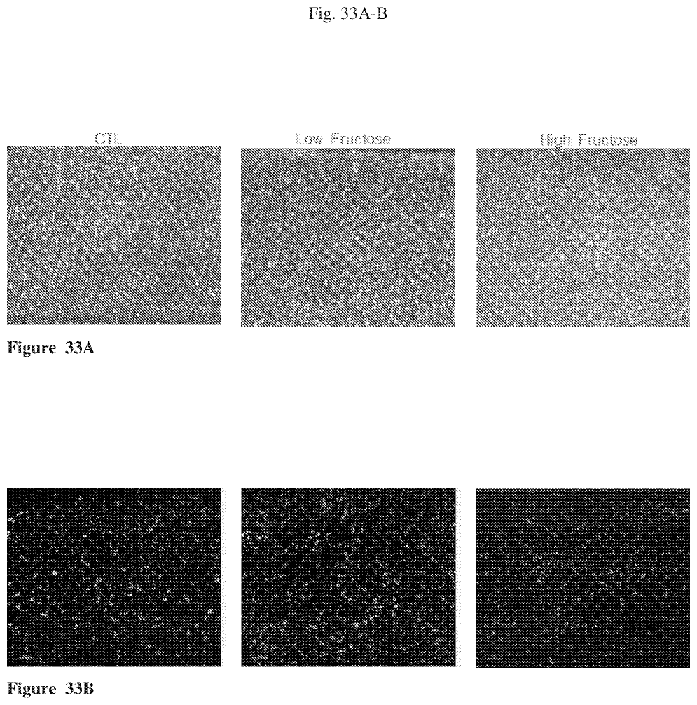

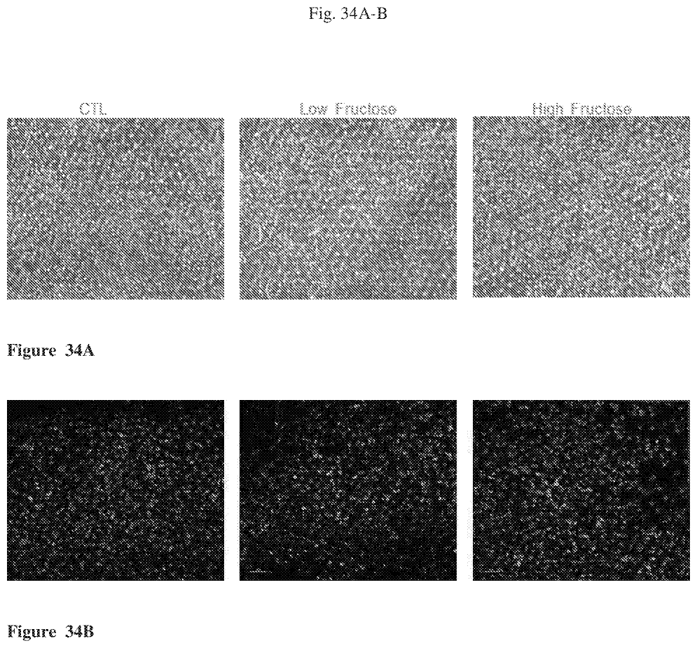

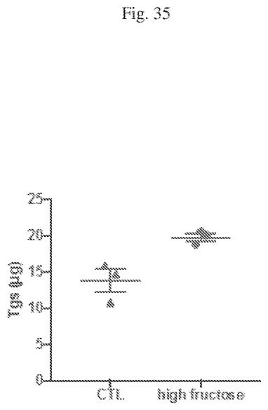

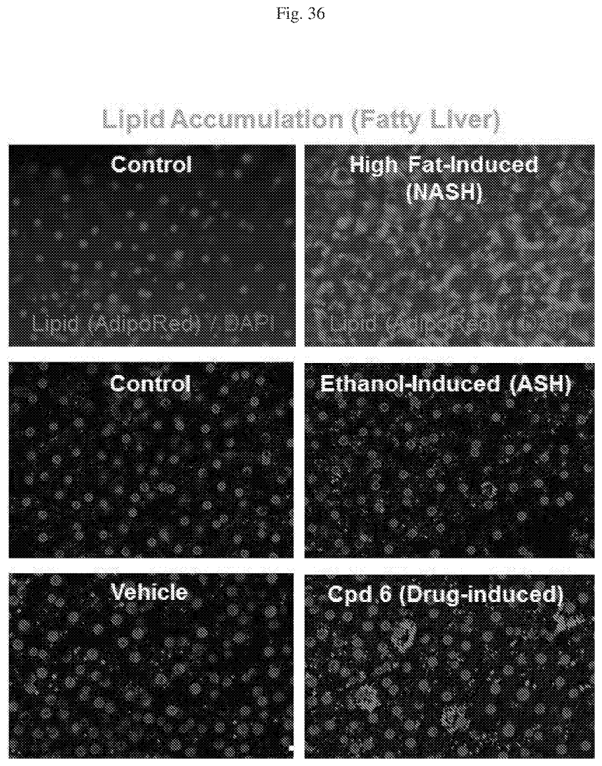

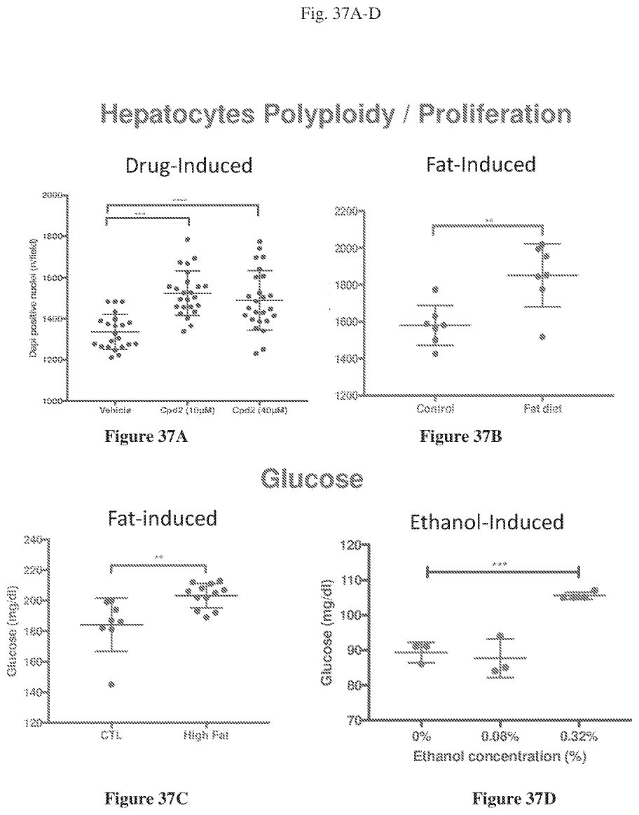

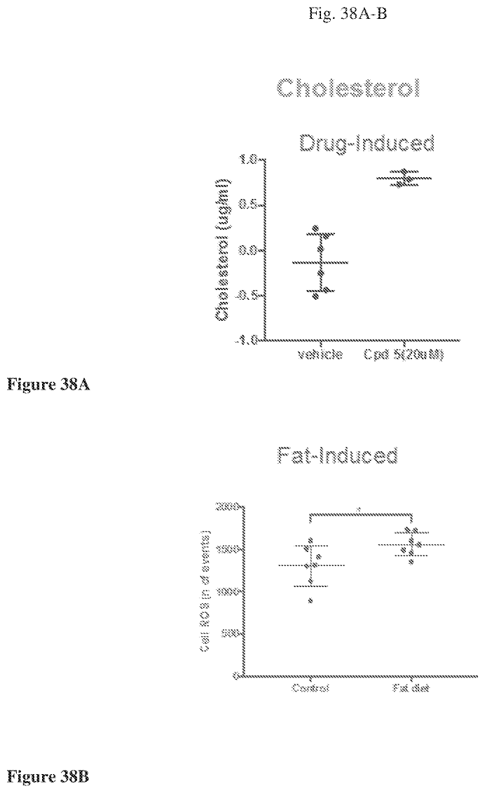

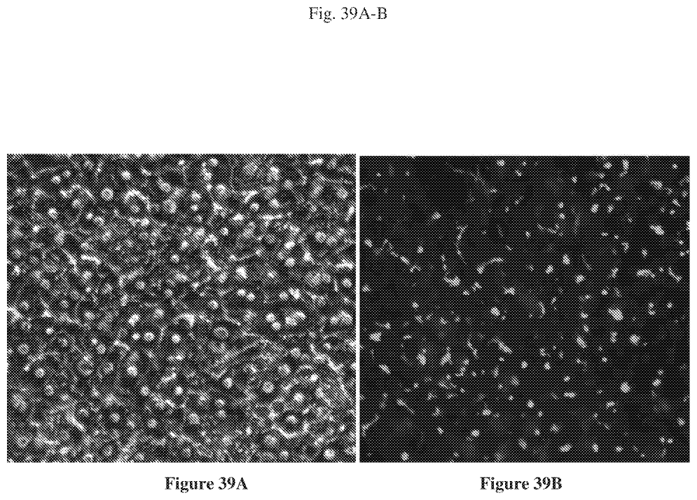

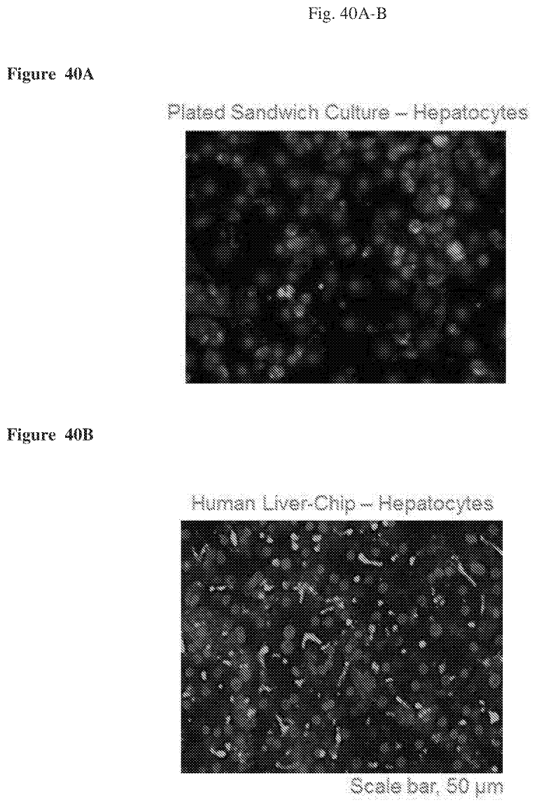

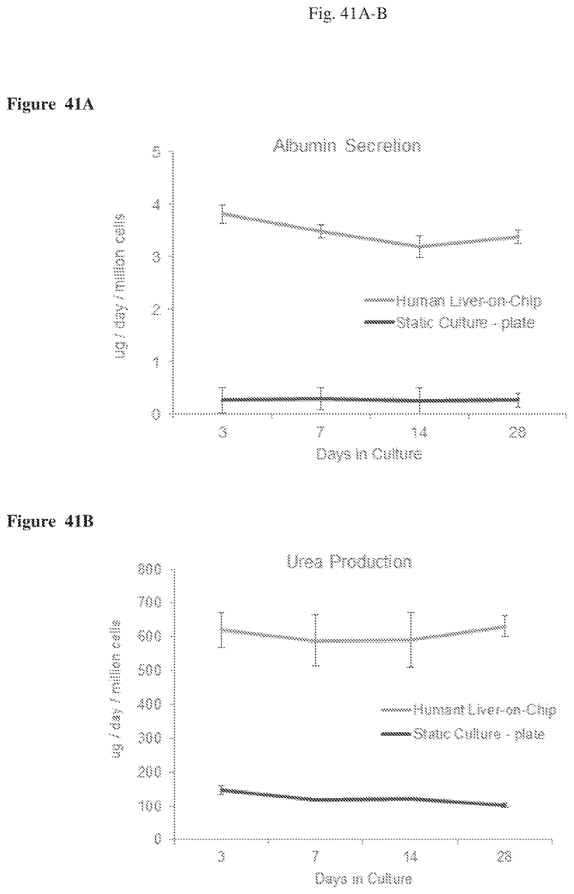

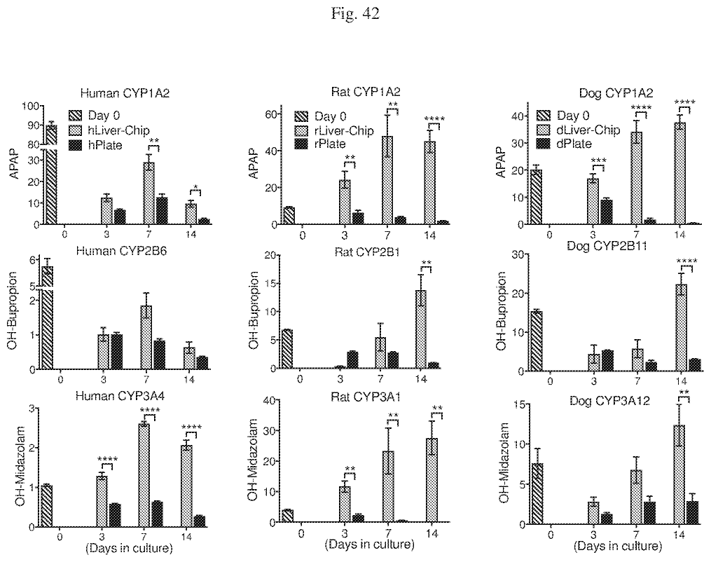

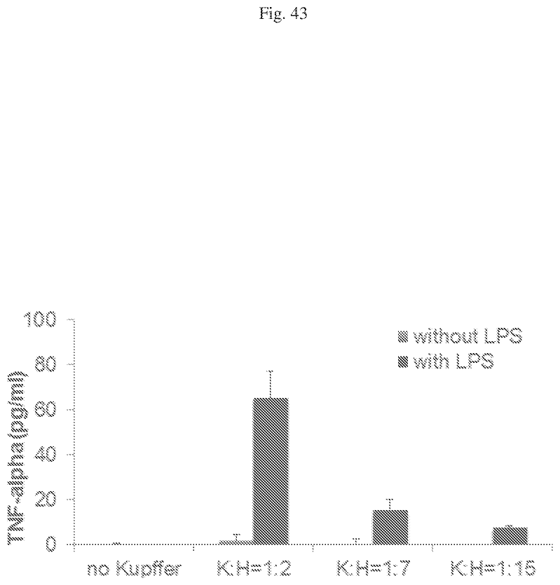

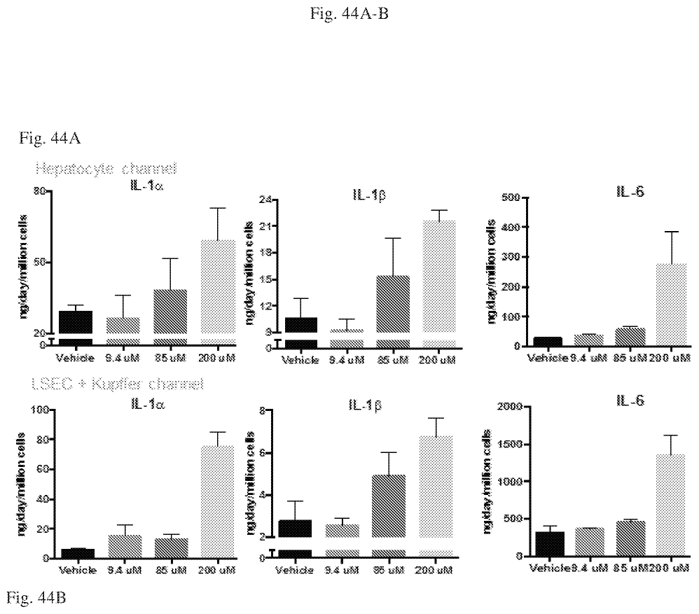

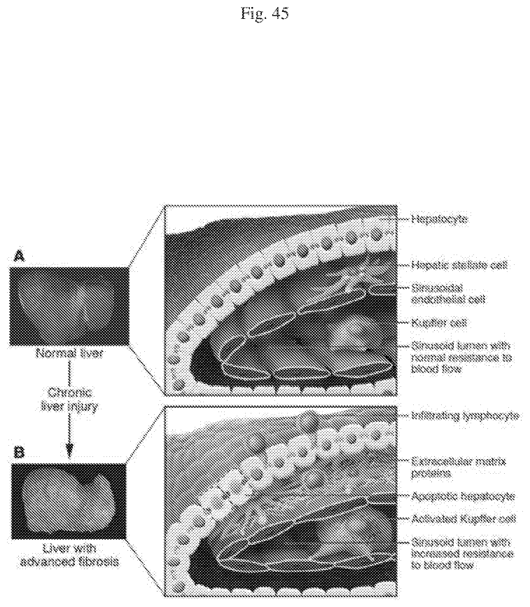

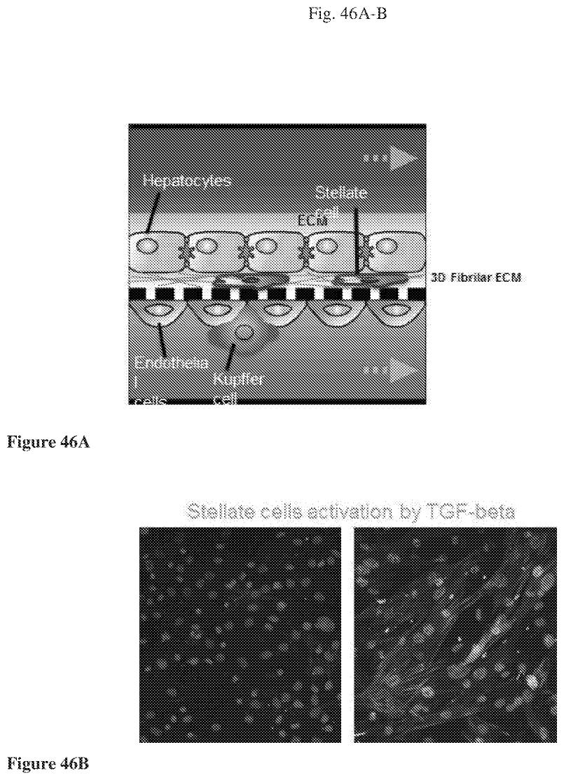

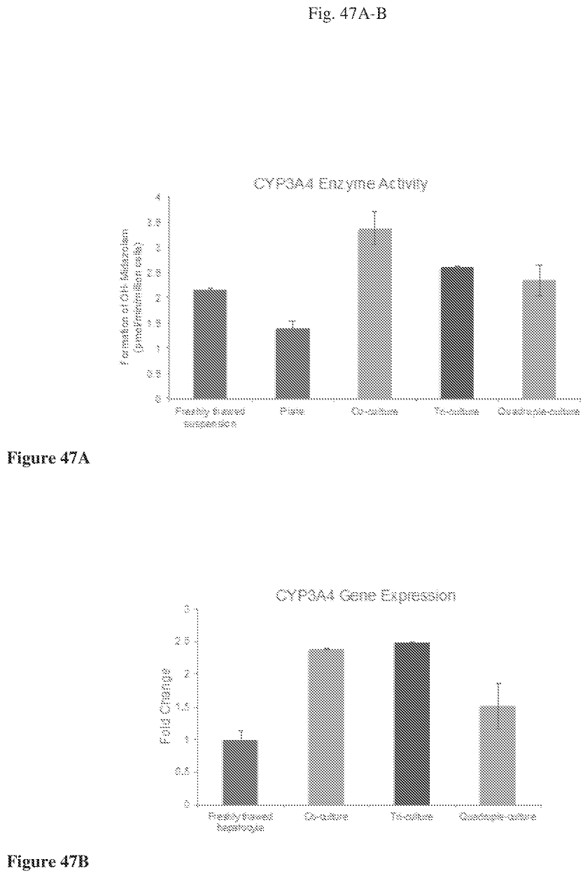

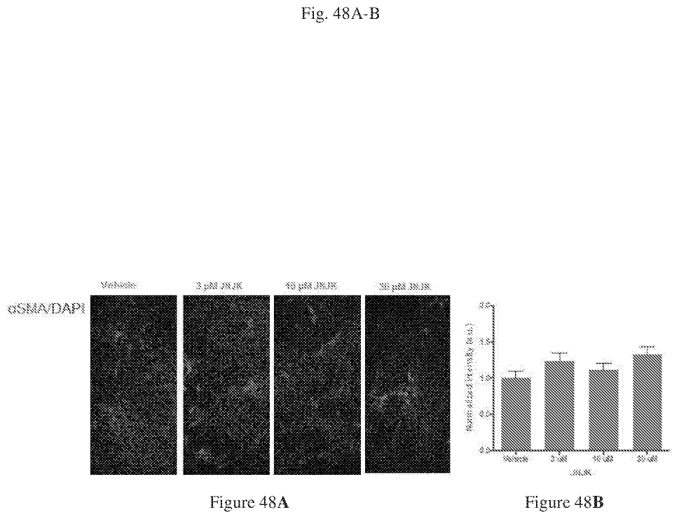

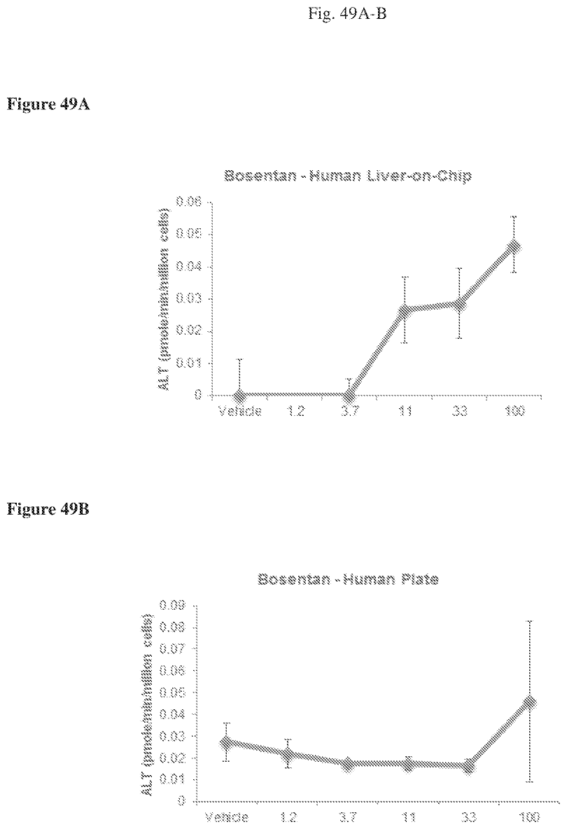

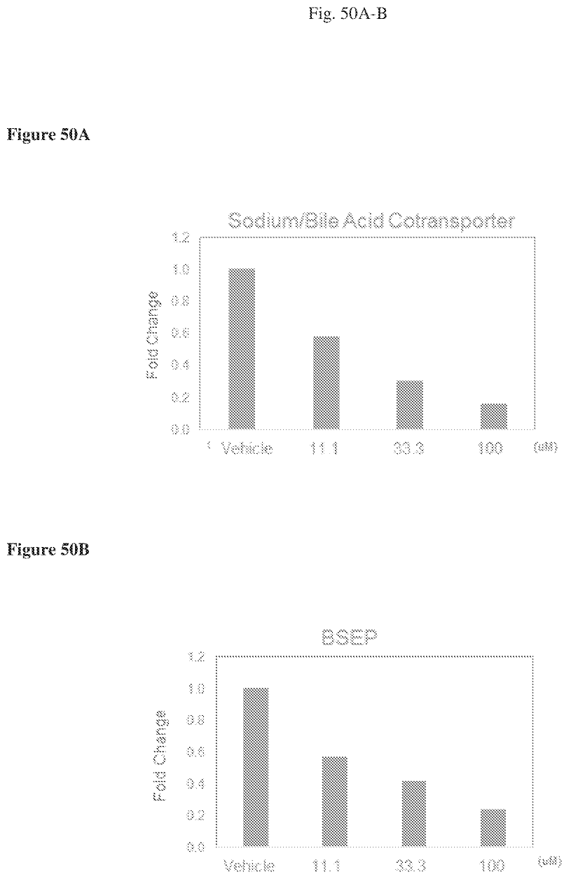

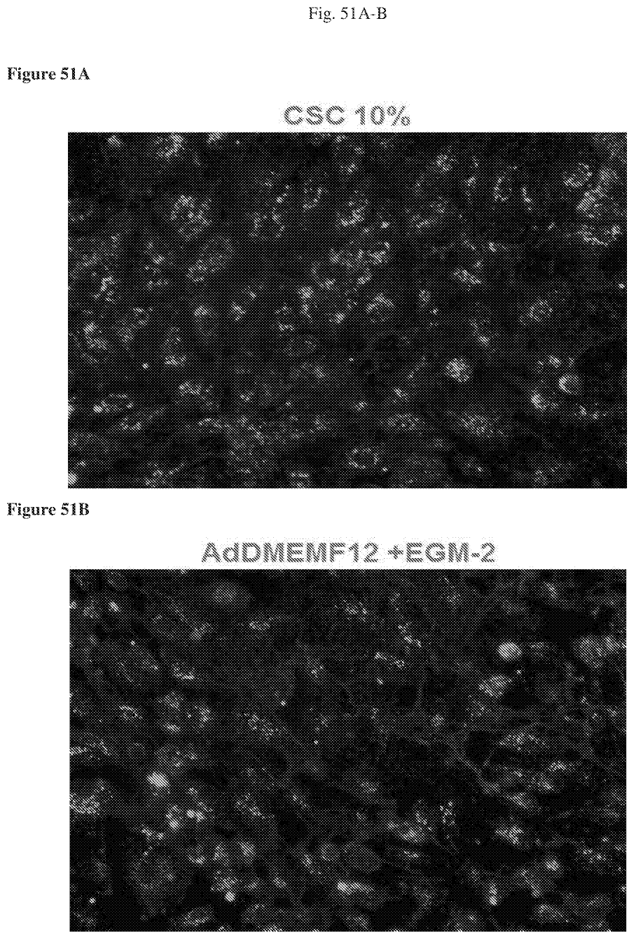

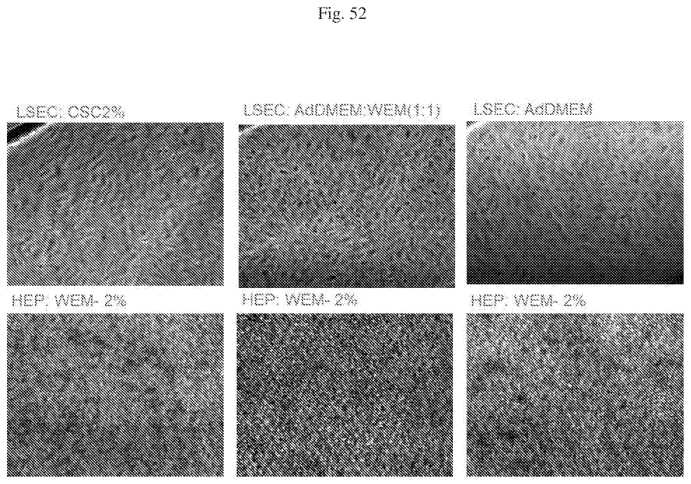

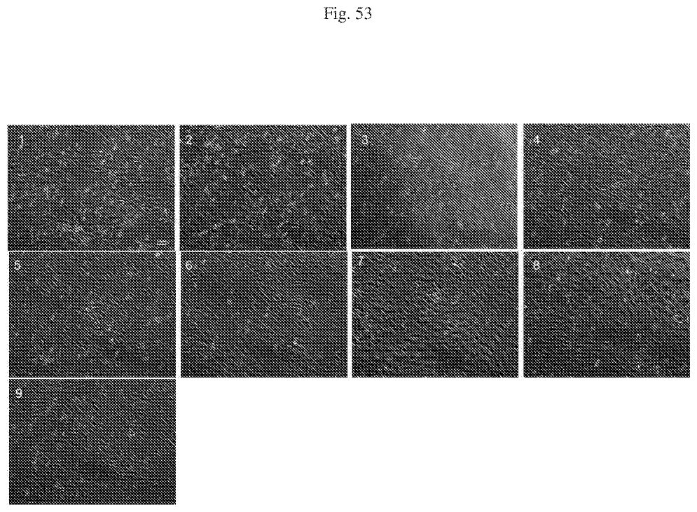

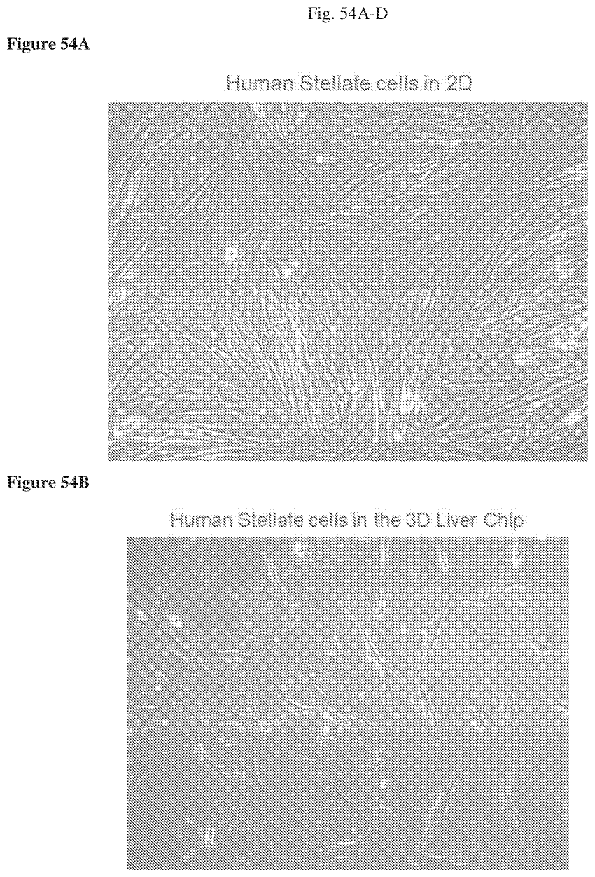

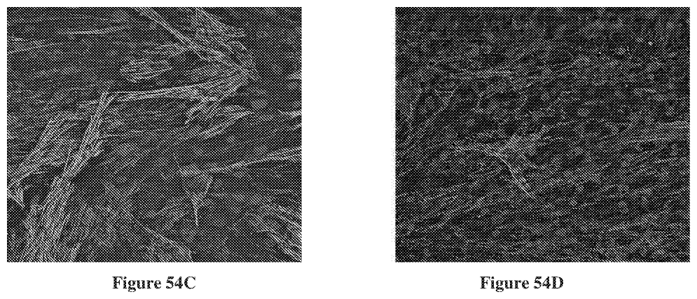

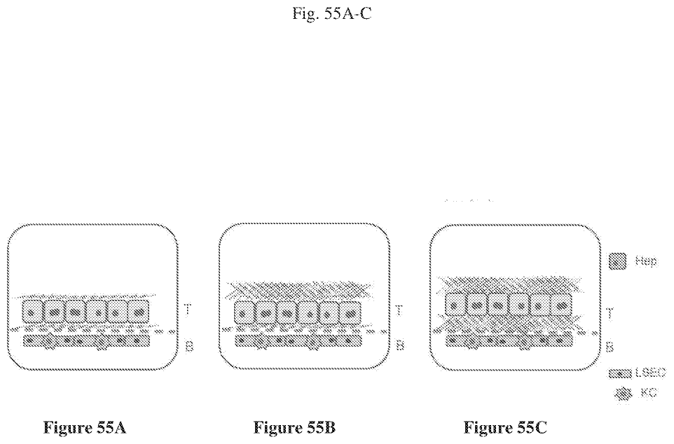

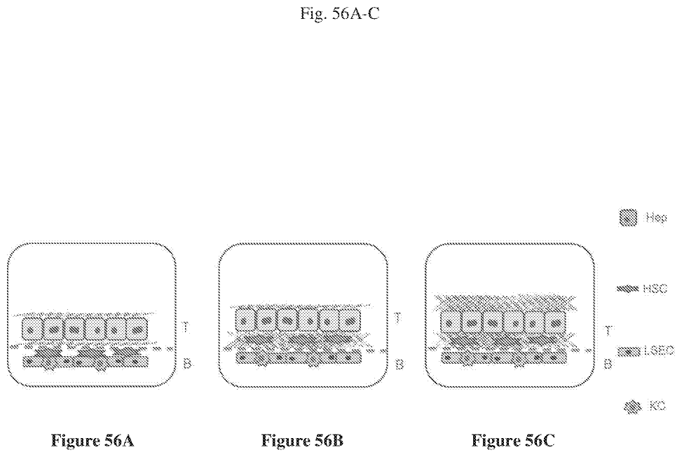

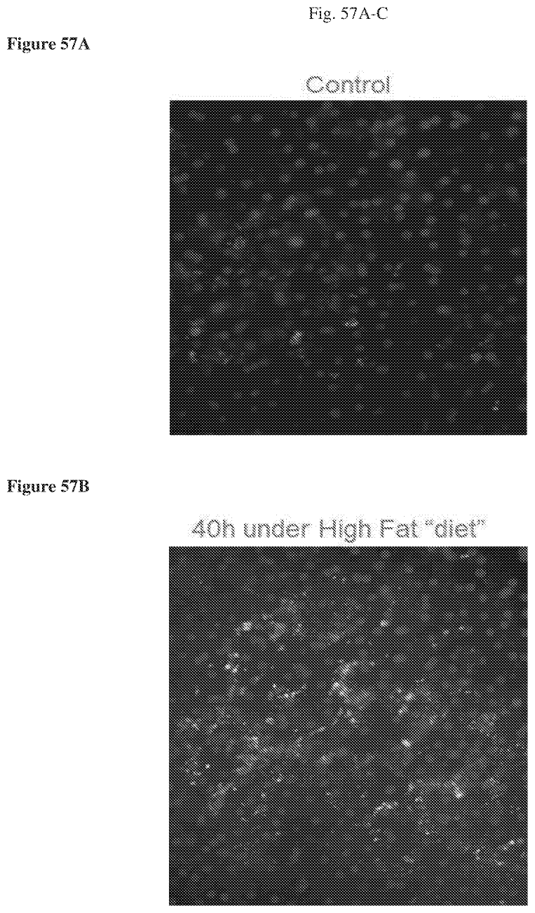

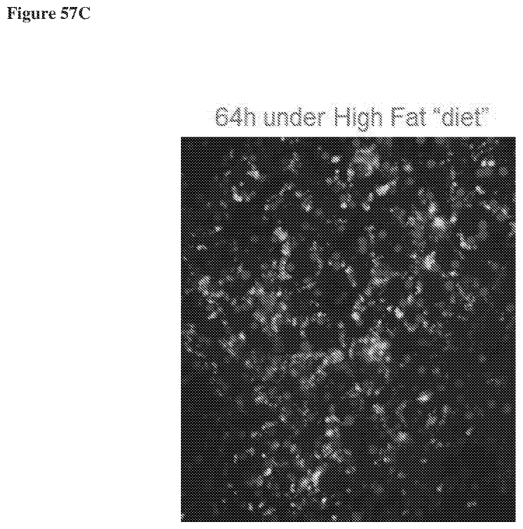

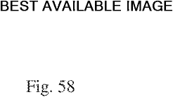

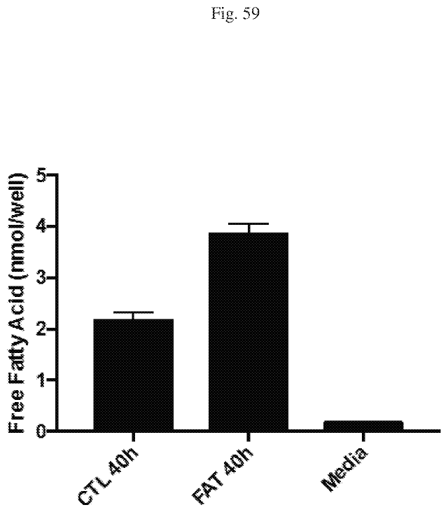

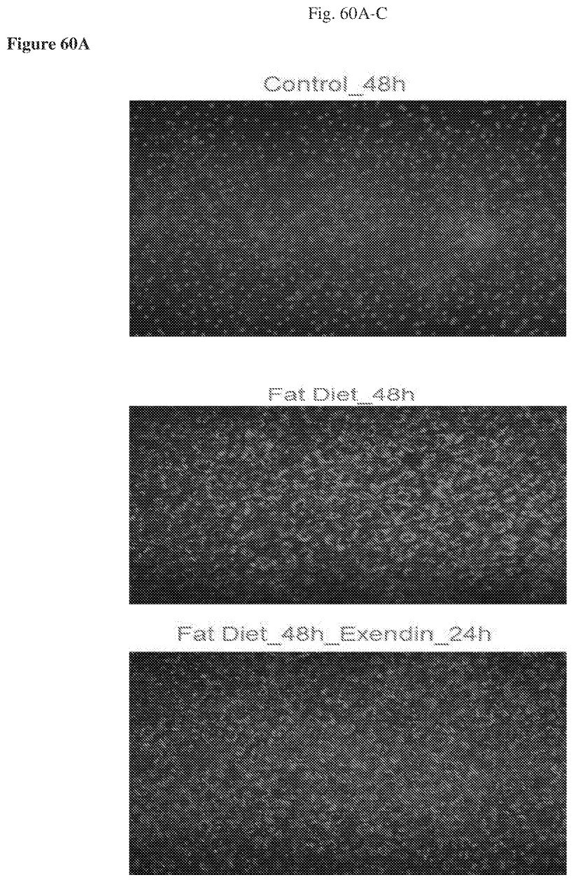

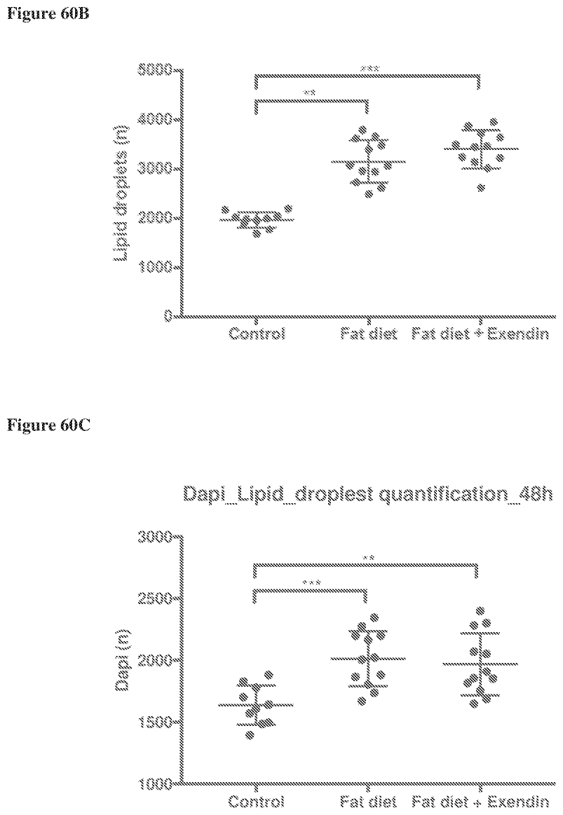

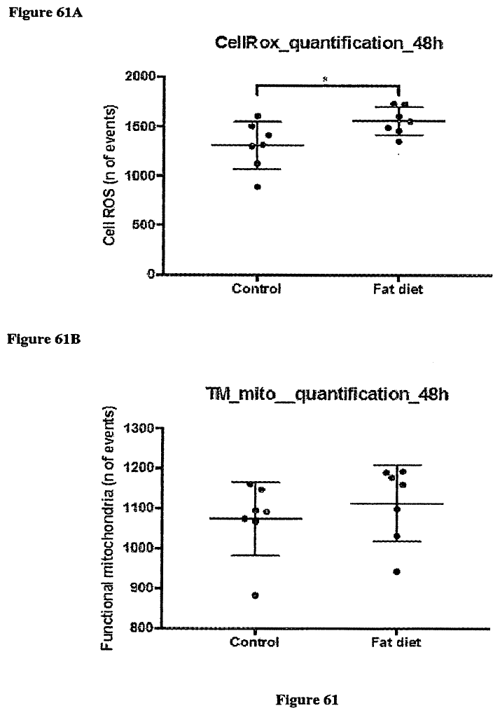

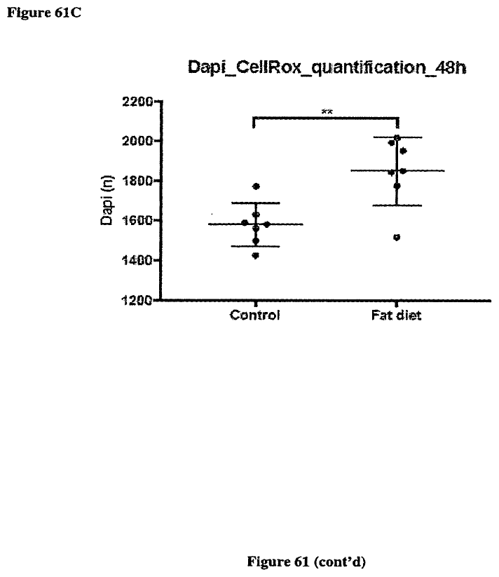

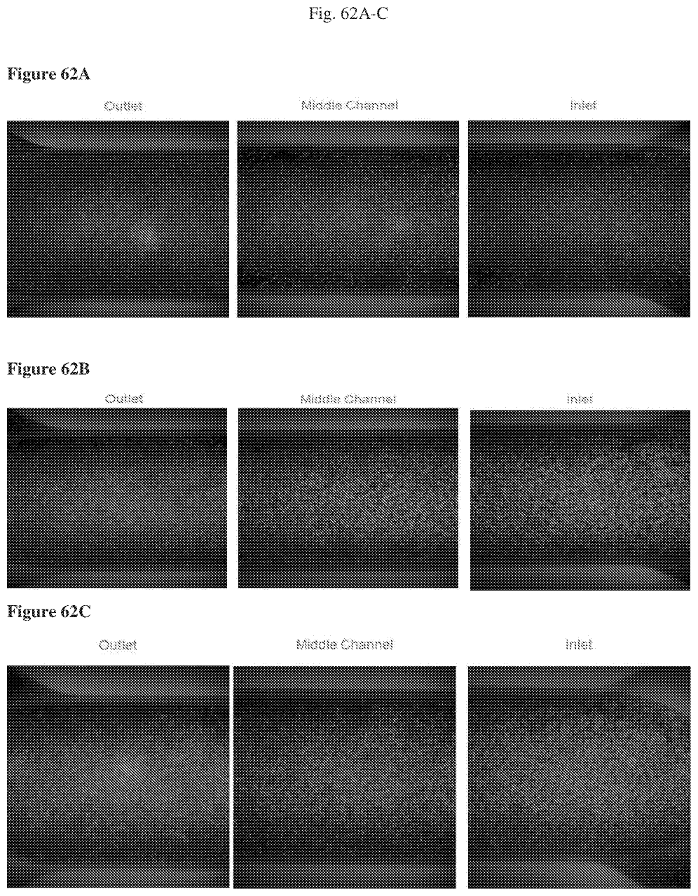

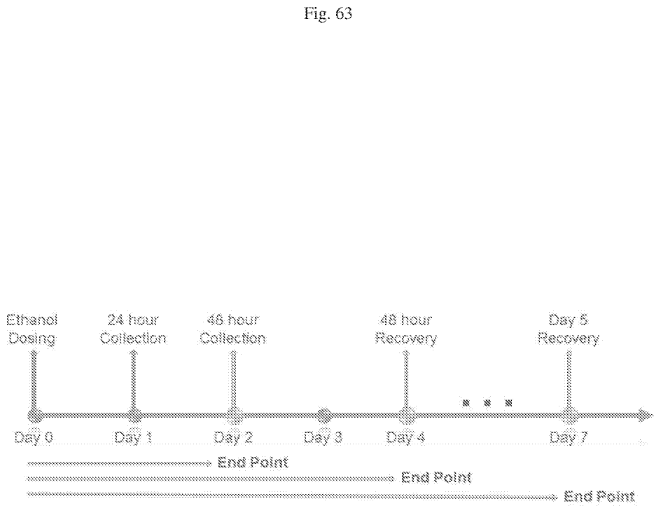

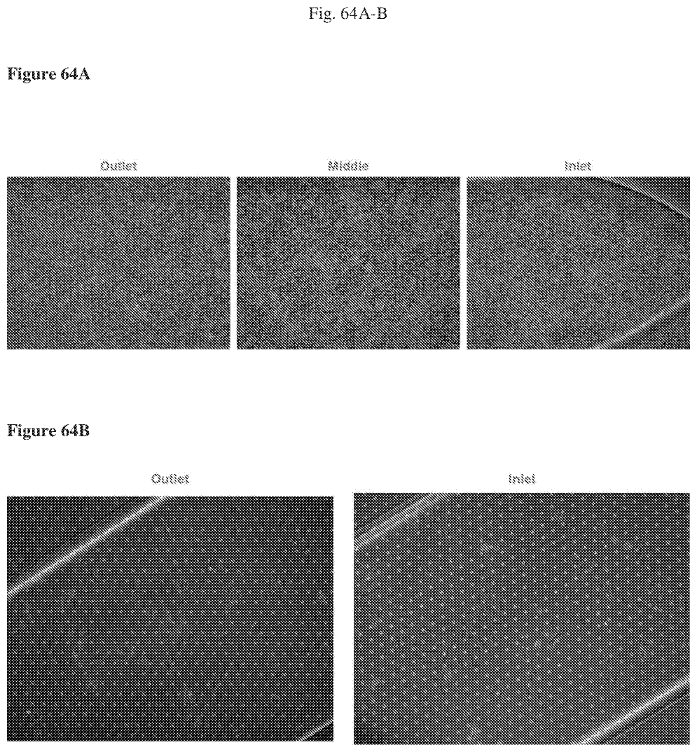

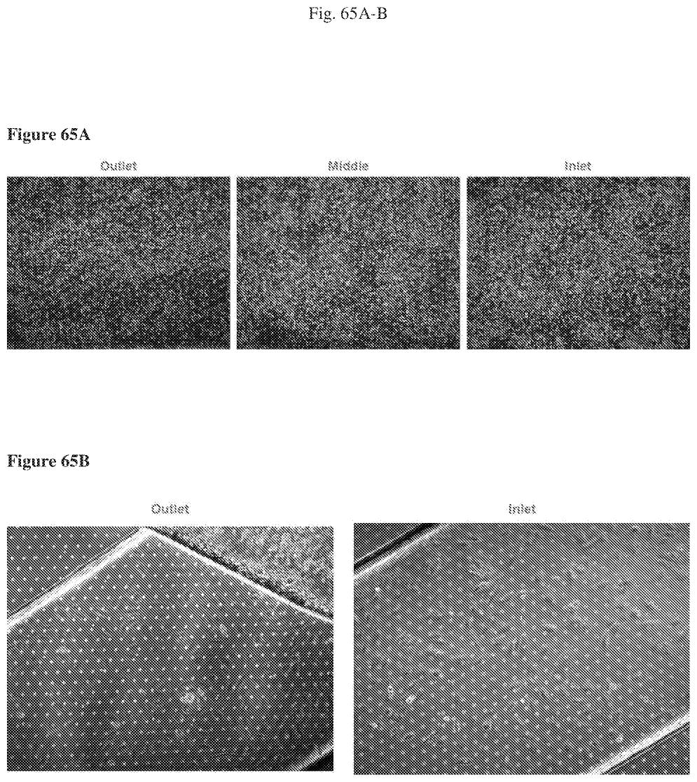

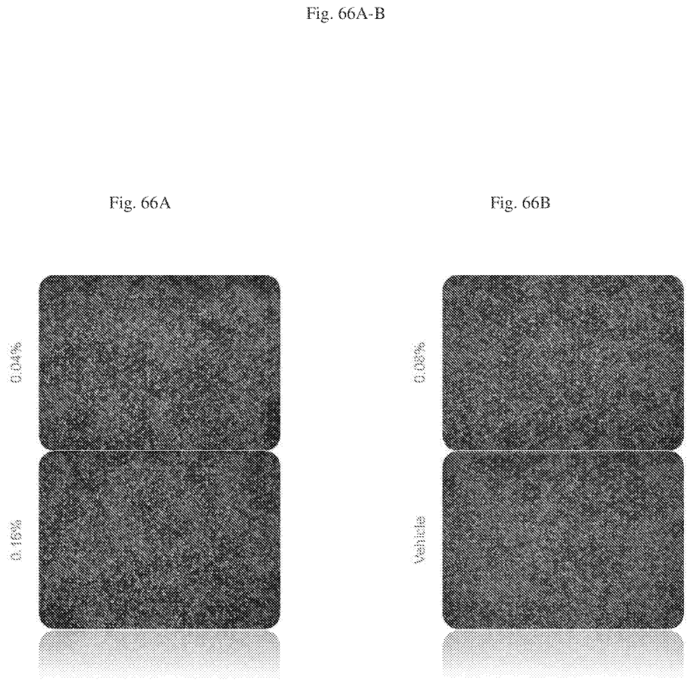

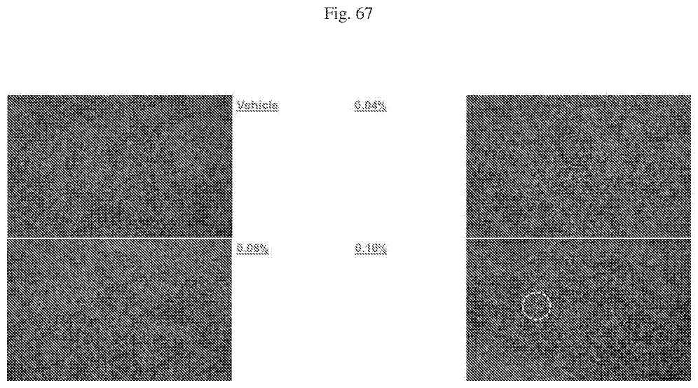

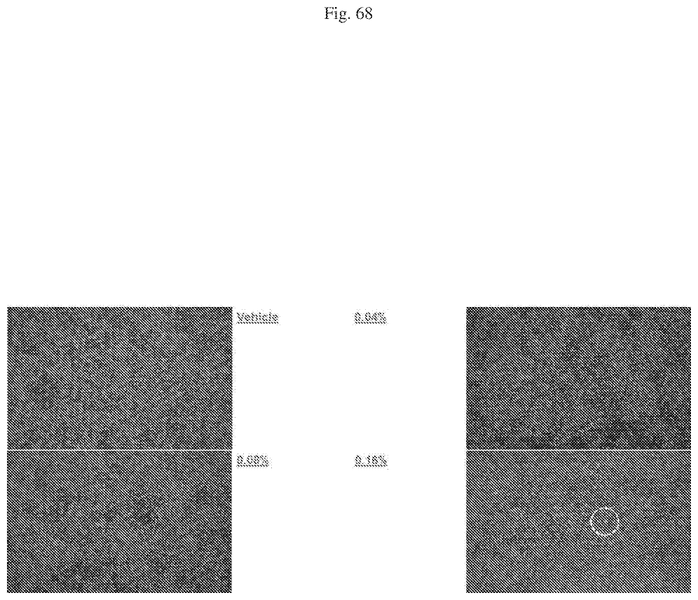

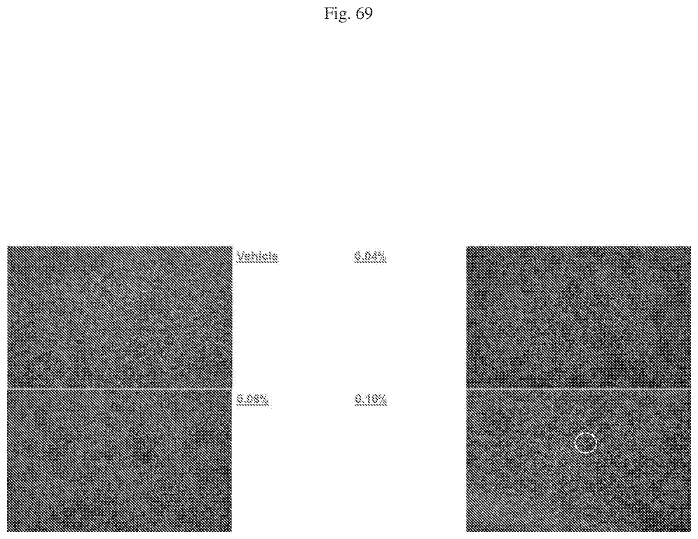

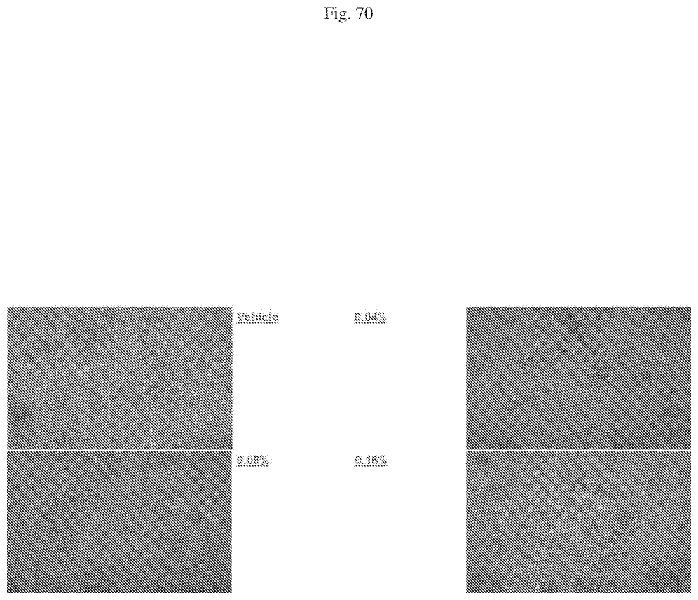

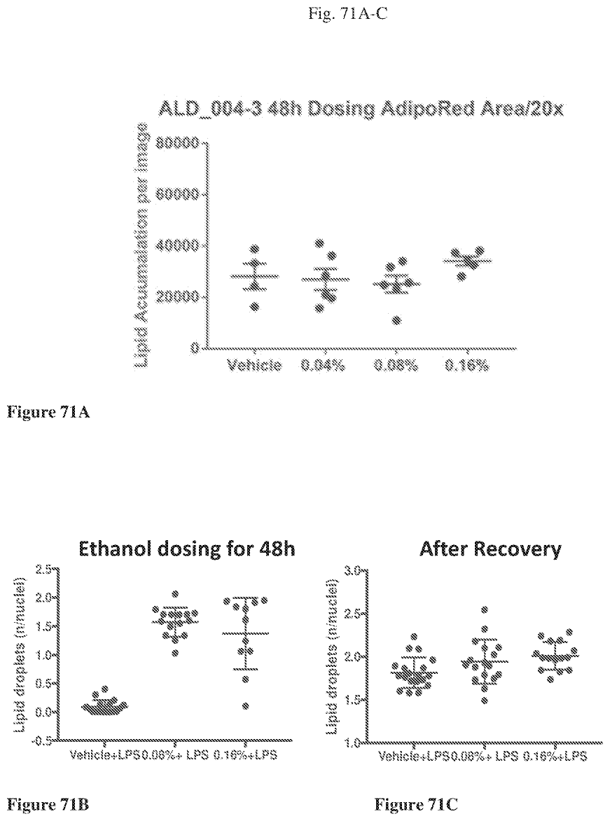

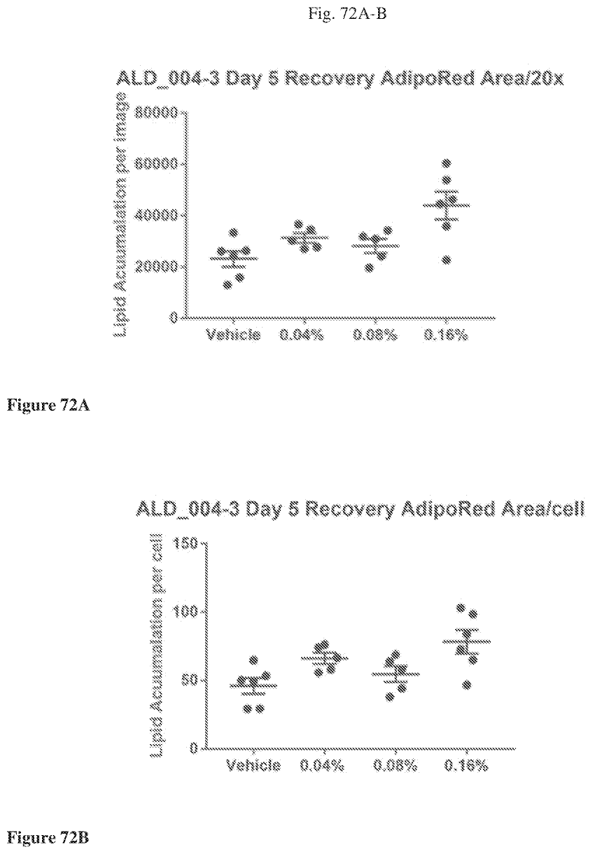

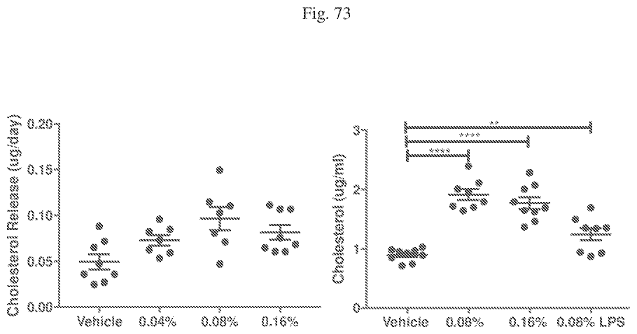

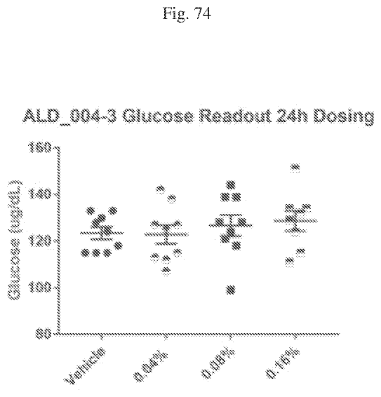

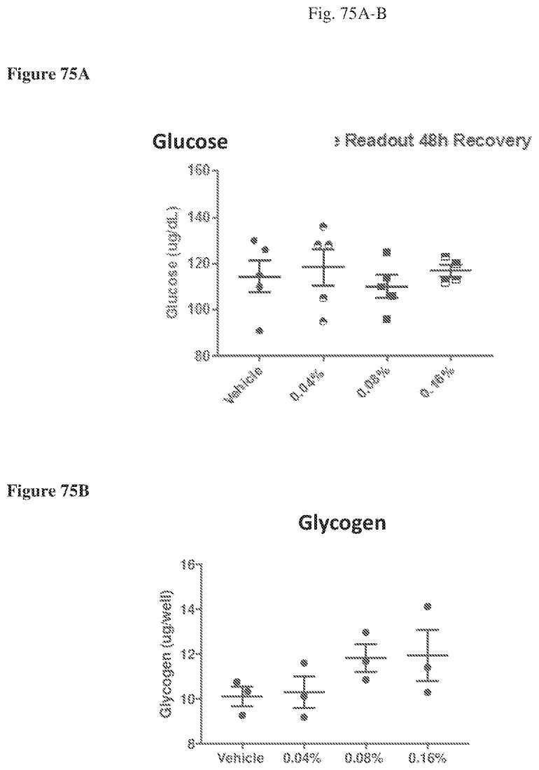

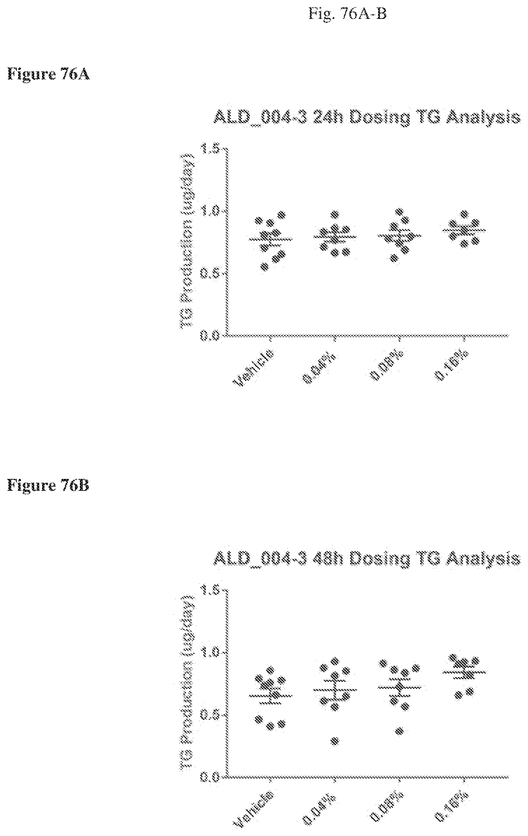

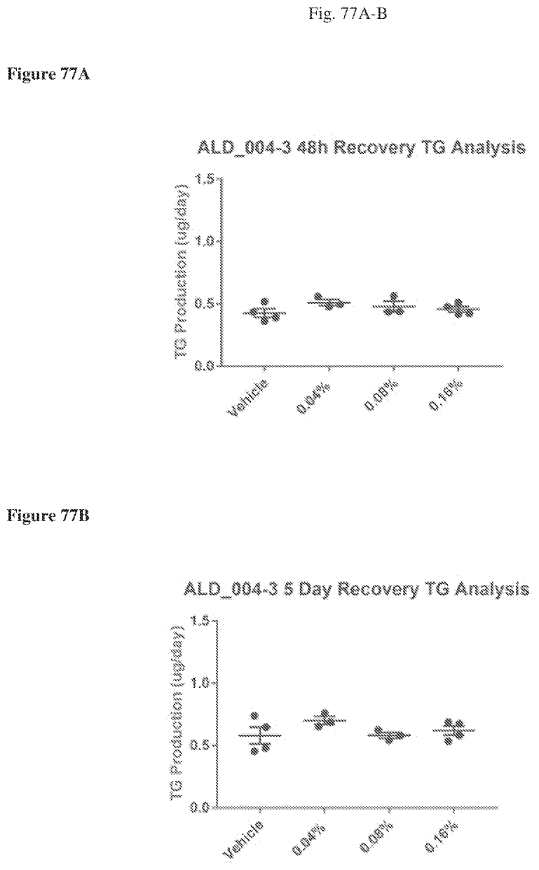

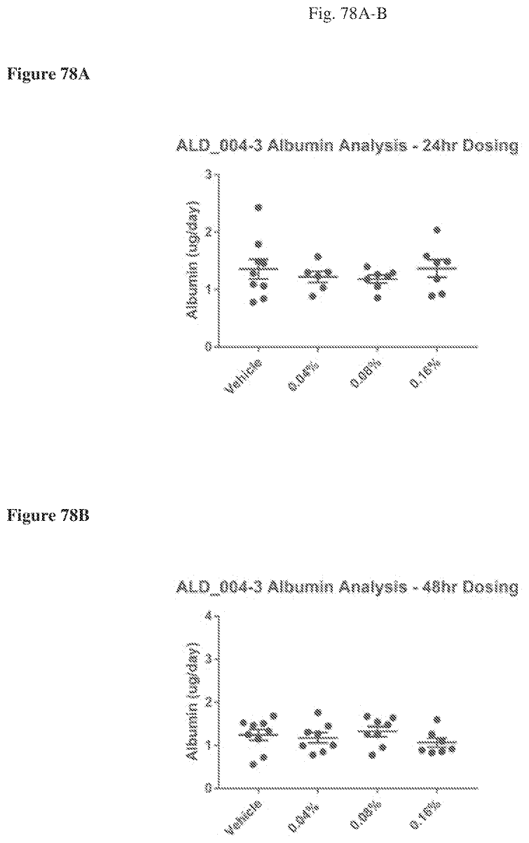

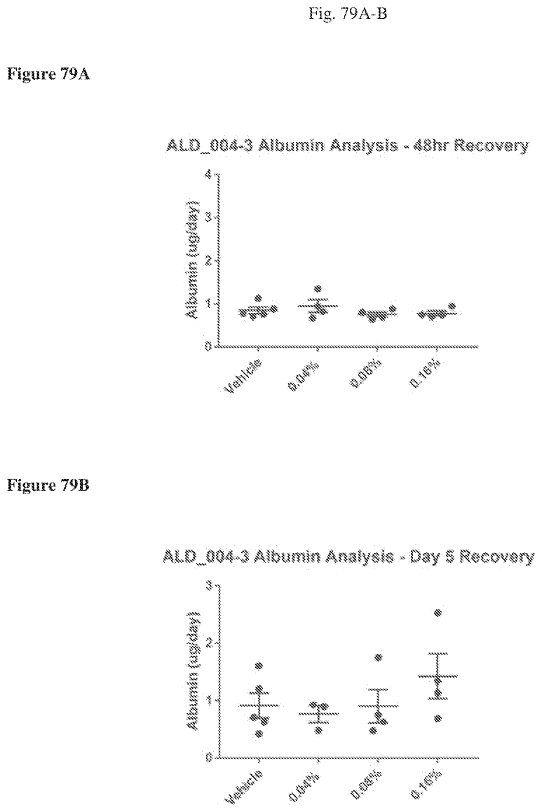

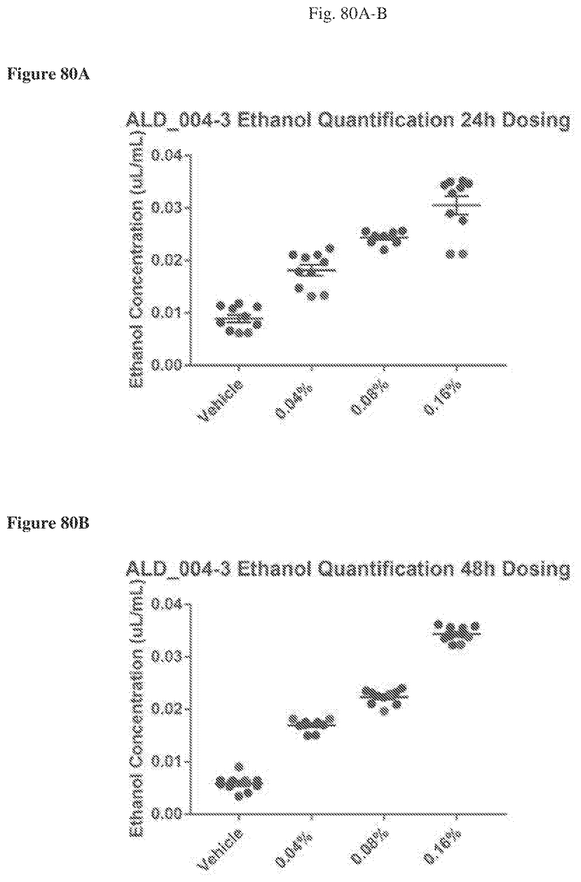

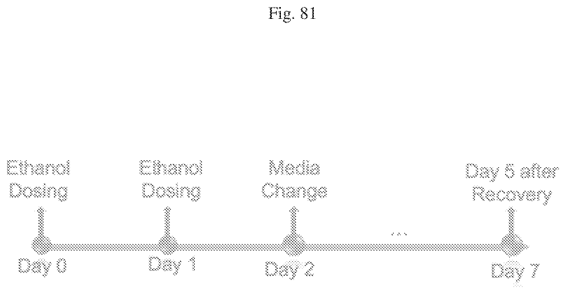

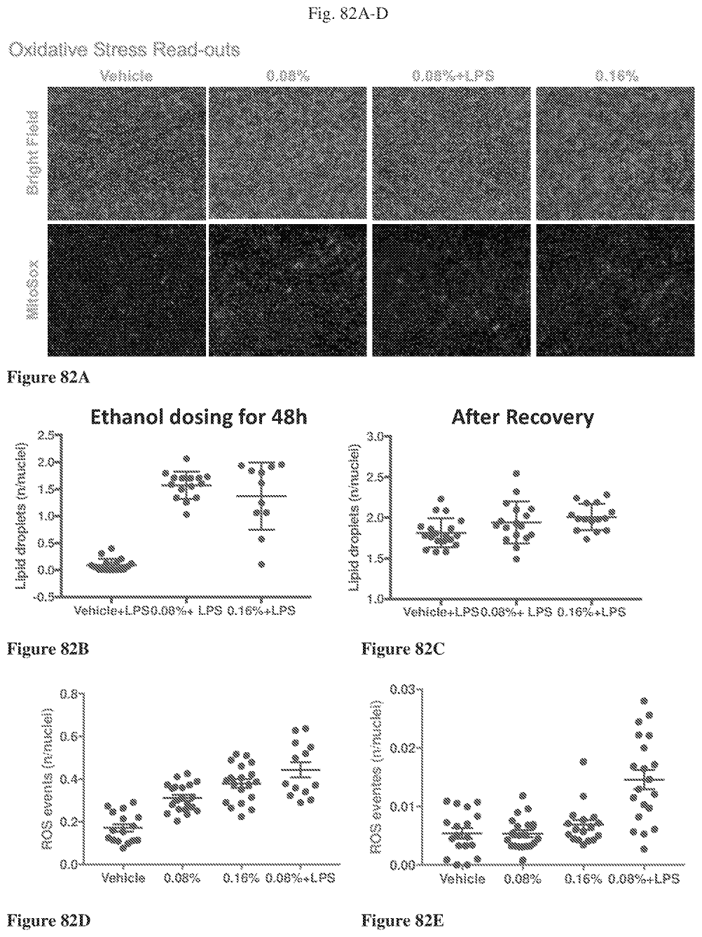

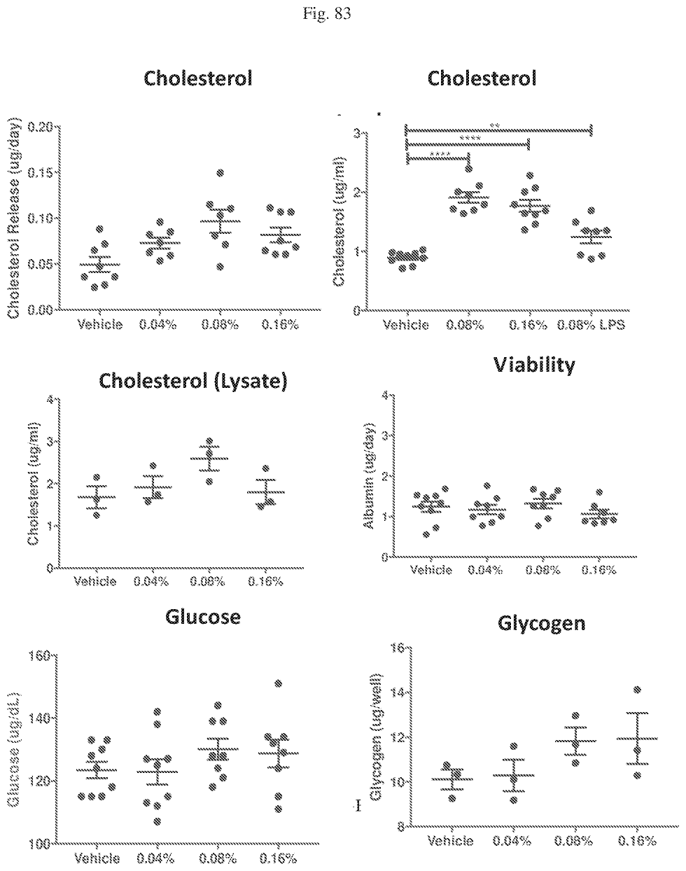

NAFLD may be experimentally induced by administration of a high fat diet, either in vivo to a subject, or in vitro to co-cultured cells (e.g., hepatocytes and LSECs). For example, a co-culture of hepatocytes and liver sinusoidal endothelial cells (LSECs) cultured on a hepatocyte microchip were exposed to a high fat diet comprising of oleic acid (0.1 to 0.5 μM) for up to two days. Accumulating fat droplets within these cultured hepatocytes were identified with AdipoRed® or NileRed® staining. Furthermore, these cultured LSECs were seen to have developed an injury morphology as seen by bright field imaging. See, FIG. 15. These data show that the presently disclosed hepatocyte microchip can be used to model lipid homeostasis dysregulation. Notably, the hepatocyte microchip system easily shows the progressive nature of lipid droplet accumulation as compared between the control, 24 hours of high fat exposure and 48 hours of high fat exposure. See, FIGS. 16A-C. Additionally, the number of lipid droplets were quantitated as well as the accompanying glucose level was determined. See, FIGS. 17A-B. These data show that under a high fat condition a significant increase of lipid droplets accumulation is observed inside the co-cultured hepatocytes. Furthermore, extracellular glucose concentration was significantly higher under the high fat diet condition. An evaluation of mitochondrial function suggested that the two day high fat diet did not result in any significant reduction in cell viability. See, FIG. 18A. Oxidative stress markers, however, were observed to be elevated during the two day high fat diet administration as shown by an increase in reactive oxygen species (ROS). See, FIG. 18B. Photomicrographs of LSEC mitochondrial function correlate in a positive manner with twenty-four (24) hours of a high fat cell diet. See, FIG. 18C. Photomicrographs of hepatocyte mitochondrial function correlate in a positive manner after twenty-four (24) hours of a high fat cell diet. See, FIG. 18D. Similar results were seen in both the LSECs and hepatocytes after forty-eight (48) hours of a high fat cell diet. See, FIG. 18E-F. Both free fatty acids and cholesterol concentrations were also observed to increase. See, FIG. 18G-H. Potential hepatocyte proliferation or chromosomal effects (e.g., polyploidy) were observed as shown by an increase in the number of nuclei. See, FIG. 19. These data show that the presently disclosed hepatocyte microchip system sustains health under physiological levels of insulin, glucose and fat. This is superior to previously reported for conventional cell culture systems that require glucose and insulin concentrations above the human physiological range and steatotic markers are induced by not physiological levels of fat. The high fat diet exposure between 24-48 hours was also performed with and without the insulin-secretion agonist, Exendin. See, FIG. 60A. A quantitative image analysis shows that exendin demonstrated no effect on lipid droplet accumulation, glucose levels. See, FIGS. 60B, 62C, 17A-B.

NASH may be experimentally induced by administration of a high fat diet, either in vivo to a subject, or in vitro to tri-cultured cells (e.g., hepatocytes, LSECs and Kupffer cells). For example, a tri-culture of hepatocytes, liver sinusoidal endothelial cells (LSECs) and Kupffer cells were cultured on a hepatocyte microchip and exposed to a high fat diet comprising oleic acid. Subsequently, LPS (1 μg/ml ) was added to the high fat diet media to induce a Kupffer cell-mediated inflammatory response. As compared to control (FIG. 20A), the exposure of oleic acid to the tri-culture results in a significant increase in observable fat droplets in the hepatocytes. See, FIG. 20B. When oleic acid exposure is supplemented with LPS the observance of stained lipid droplets decreases. See, FIG. 20C. The administration of LPS to the tri-culture (without any high fat diet oleic acid), the observation of lipid droplets is not different from control levels. See, FIG. 20D. A quantitative scatterplot of these tri-culture lipid droplet accumulation shows the relative statistical significance between the respective treatment groups. See, FIG. 21A. On the other hand, extracellular glucose was observed to increased in all treatment groups relative to control See, FIG. 21B. Further analysis found that the location of the tri-cultured cells in the NASH model showed differential lipid droplet accumulation after 48 hours of a high fat diet of oleic acid. For example, heat maps of the relative distribution of lipid droplet accumulation between hepatocyte cells that are proximal to the channel inlet have a higher density of lipid droplet accumulation as compared to hepatocyte cells that are proximal to the channel outlet. See, FIG. 22A cf FIG. 22B. These data suggest that the tri-cultured hepatocytes are differentiating into “metabolic zones” that may resemble an in vivo liver cell architecture. See, FIG. 23.

NASH may be experimentally induced by administration of a high fat diet, either in vivo to a subject, or in vitro to quad-cultured cells (e.g., hepatocytes, LSECs, Kupffer cells and hepatic stellate cells). The quad-cultured cells is designed to recapitulate the cellular architecture of an in vivo liver tissue. See, FIGS. 24A-B and FIG. 25. For example, a quad-culture of hepatocytes, liver sinusoidal endothelial cells (LSECs), Kupffer cells and hepatic stellate cells (HSCs) were cultured on a hepatocyte microchip and exposed to a high fat diet comprising oleic acid. Subsequently, LPS (1 μg/ml ) was added to the high fat diet media to induce a Kupffer cell-mediated inflammatory response. In order to determine the best environment HSCs were add to the chip embedded within different extracellular matrix (ECM) including, but not limited to, Matrigel® (0.25 mg/ml), Matritek® (0.1-0.4 mg/ml), or collagen (0.5-1 mg/ml). Some of these ECMs were capable of supporting HSC 3D morphology in the microfluidic Emulate design however activation of HSC were lower on the Collagen I 0.5 mg/ml condition suggesting that the ECM composition does play a role on the HSC maturation state (low activation high vitamin A storage). See, FIGS. 26A-D. Although it is not necessary to understand the mechanism of an invention, it is believed that the activation of HSC to produce inflammation comprises a transdifferentiation of quiescent, vitamin-A-storing HSC cells into proliferative, fibrogenic myofibroblasts. The quad-culture NASH model was also observed to create HSC bile caniculi and sinusoids that are morphologically similar to those seen in vivo. See, FIGS. 27A-B cf FIGS. 28A-D. These data show that the quad-culture NASH model as disclosed herein provides a recapitulated hepatic architecture that reliably functions as does an in vivo liver. For example, following the exposure of the quad-culture NASH model to a high fat diet comprising oleic acid followed by LPS administration the liver disease stage may be assessed by measurement of the following: i) HSC activation is determined by detection of alpha-smooth muscle actin (αSMA); ii) hepatocyte viability and functionability is determined by measurement of albumin, LDH and bile canaliculi network; iii) cytokine production is determined by measurement of IL-1, IL-6, TGF-beta and/or TNF-α;

For example, tacrine was used to stimulate cytokine expression in hepatocytes culture in the present disclosed microchip. Tacrine stimulation resulted in the accumulation of IL-1-α, IL-1beta and IL-6 in both the hepatocyte channel and the LSEC/Kupffer-cell channel. See, FIG. 35.

B. Fructose-Induced Liver Injury

The data presented herein demonstrate the effect of fructose on a co-culture hepatocyte microchip with either a hepatocyte culture media or an intestinal effluent media. The cell culture media effluent was analyzed for triglycerides, glycogen and glucose from both the top and bottom channels of the microchannel. See, Example 1. Cell imaging analysis of lipid droplet accumulation was performed with AdipoRed® and visualized with brightfield imaging.

The hepatocyte cells and endothelial cells were seeded and incubated as a co-culture in a hepatocyte microchip and samples were collected a specific time points. See, FIG. 32.

The specific media compositions were as follows:
Liver culture media:
   Top Channel: DMEM (LG) 0% FBS, NEAA 1:200, ITSG 1:10,000, Vitamin C & dexamethasone

Bottom Channel: LG CSC

To each media, either a 1:1 glucose:fructose ratio (low fructose) or a 0.7:1.4 glucose:fructose ratio (high fructose) is added.

The liver culture media had no effect on hepatocyte viability but decreased hepatocyte cell number and increased hepatocyte cell size after 48 hours of incubation in either low or high fructose concentrations. See, FIGS. 33A-B.

Sampling of the microchannel effluent reflected a similar pattern in regards to glucose concentrations. Glucose was observed to increase after 48 h incubation of hepatocytes with a fructose media. In contrast, no difference observed in hepatocyte cells incubated either with or without fructose in the intestinal effluent culture media. See, FIGS. 33A-B.

Sampling of the microchannel effluent reflected a different pattern in regards to cholesterol concentrations. Fructose failed to significantly increase cholesterol levels in either liver media or intestinal effluent media after 24 hours, 48 hours or 72 hours of incubation. See, FIGS. 34A-B.

The determination of extracellular glycogen levels demonstrated a dose-dependent decrease in response to either low or high fructose using both a liver media or an intestinal effluent media after 48 hours of incubation. See, FIG. 35. No significant differences were seen after 24 or 72 hours of incubation

The determination of triglyceride levels demonstrated a dose-dependent increase in response to either low or high fructose using a liver media after 48 hours of incubation. No significant differences were seen after 24 or 72 hours of incubation or in the presence of an intestinal effluent media. See, FIGS. 34A-B.

In conclusion, the liver media conditions demonstrated small but significant changes on some hepatic metabolic markers (glucose, glycogen and TG) measured from Liver-Chip effluent co-cultured with fructose. Although it is not necessary to understand the mechanism of an invention, it is believed that the data presented herein may be responsive to initial hepatic metabolic marker concentrations by fructose addition to the media. Modifications of cell culture media to control these hepatic biomarkers (e.g. lower insulin range or glucagon addition) may express bigger changes.

### III. Microfluidic Liver Tissue Systems

The above described unmet medical need for the treatment of ALD is in part due to lack of human-relevant model systems to study the effect of alcohol on liver development and regeneration. In recent years, human-relevant microphysiological systems have been demonstrated to be a tool for modeling human physiology in vitro. In one embodiment, the present invention contemplates a microfluidic solid substrate system for the modelling of multiple ALD stages, including but not limited to, alcoholic fatty liver, ASH, alcoholic fibrosis and non-alcoholic liver diseases. Although it is not necessary to understand the mechanism of an invention it is believed that this microfluidic solid substrate system can identify the points of alcoholic toxicity reversibility and/or irreversibility.

The lack of a suitable animal model has been an impediment to deeper study of ALD experimentally, and is one of the reasons for the suboptimal research to identify novel ALD bio-markers. In that context the development of a human and physiological relevant microphysiological system disclosed herein can facilitate progress in ALD research and related drug development.

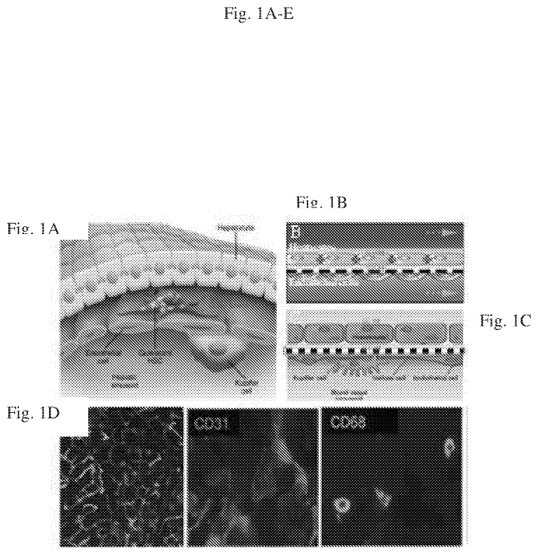

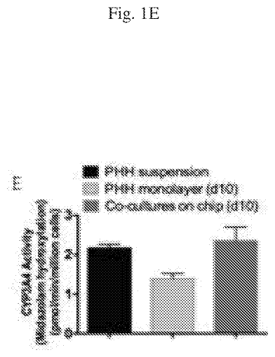

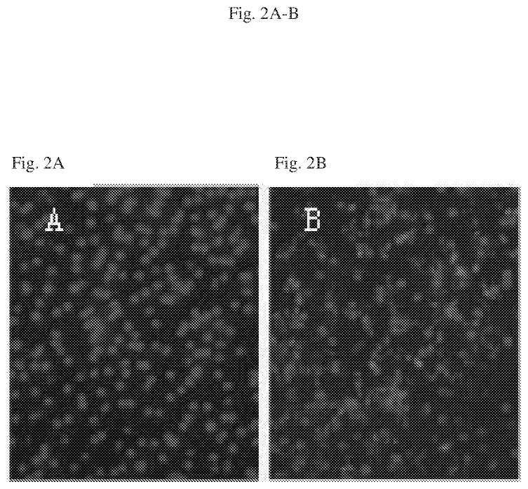

In some embodiments, the microfluidic liver system contemplates a liver microphysiological system using primary human cells and possessing liver sinusoid architecture to mimic human and physiological ALD progression in vitro. Such a system supports hepatic function that is indistinguishable from an in vivo environment. See, FIG. 1A. Although it is not necessary to understand the mechanism of an invention, it is believed that the value of such studies is an ability to measure hepatocyte function in a complex system superior to the use of hepatocytes in monoculture and is more human-relevant than in vivo animal models. Hepatic cell monocultures are known to be limited by the loss of metabolic function and phenotype. Furthermore, physiological levels of many proteins such as P450 enzymes fall rapidly during primary hepatocyte culture and may not be accurately represented by tumor-derived cell lines such as HepG2.

Animal models, although informative, have major translational limitations due to differences in ethanol metabolism. Microfluidic tissue testing systems as disclosed herein may comprise human hepatic primary cells that are derived from ALD patients. Microfluidic tissue testing systems as disclosed herein can deliver different ranges of blood alcohol concentration (BAC) thereby mimicking distinct levels of ethanol consumption including, but not limited to, moderate, binge and heavy alcohol users. See, Table I.

TABLE I
Classification of Alcohol Consumption.
Ethanol exposure in alcohol consumption categories

| | MODERATE | BINGE | HEAVY |
|---|---|---|---|
| BAC | 0.02% to 0.05% | 0.08% | 0.08% to 0.3% |
| Exposure time | 2 h | 2 h to 6 h | 6 h-48 h |
| Frequency | 1 to 3 events with | 1 to 3 | Up to 7 days |
| | intercalate | consecutive | |
| | recovery days | days | |

Most literature reporting data for ALD using in vitro models were done using non-physiological BAC (around 100 mM). That is probably necessary because of the absence of a hepatic sinusoid architecture (i.e., for example, as in a spheroid model and/or a 2D model). Furthermore, perfusion culture, which can provide the physiological microenvironment of liver, is required to allow physiological ALD development. In conclusion, the presently disclosed microfluidic liver testing system provides all these aspects as a platform for new drug development and screening for liver injury therapy and/or protection for alcohol users and former users. These systems can also support the use of cells derived from patients providing insights about patient's specific response and host genetic factors

In some embodiments, the presently disclosed microfluidic liver testing systems are microengineered cell culture modalities that contain continuously perfused chambers supporting a plurality of living primary human cells (e.g., primary human liver cells) arranged to recapitulate a tissue-level architecture in order to achieve in vivo relevant physiology. By recapitulating multicellular tissue-tissue interfaces, extracellular matrices, physicochemical microenvironments, vascular perfusion and other components of the in vivo microenvironment, the microfluidic tissue testing systems allow for a high fidelity of tissue and organ functionality not possible with conventional 2D or 3D culture systems. Bhatia et al., “Microfluidic organs-on-chips” Nat Biotechnol. 2014 August; 32(8):760-72.

These microfluidic systems also enable high-resolution, real-time imaging coupled with a capability for in vitro analysis of biochemical, genetic and metabolic activities, all in the functional context of living tissue. This technology can be used to evaluate tissue development, organ physiology and/or disease etiology. In the context of drug discovery, including safety and efficacy, this technology is especially valuable for the study of molecular mechanisms of action, prioritization of lead candidates, toxicity testing and biomarker identification. Bhatia et al., “Microfluidic organs-on-chips” Nature Biotechnology 2014, 32(8):760-772. For example, no CPD-K toxicity was observed in human hepatocyte microchips when exposed to various concentrations of JNJK. See, FIGS. 52A-B. During exposure to various concentrations of bosentan, a dose dependent increment of secreted alanine transaminase (ALT) levels was found in human hepatocyte microchips, in contrast to a lack of response in conventional plate cultures. See, FIGS. 53A-B. Bosetan exposure was also found to reduce sodium/bile acid cotransporter and BSEP transporter expression levels in a dose-dependent fashion after three (3) days of culture. See, FIGS. 54A-B.

A. Architecture of a Microfluidic Liver Tissue System

Although it is not necessary to understand the mechanism of an invention, it is believed that microfluidic systems described herein are able to characterize liver tissue responses upon exposure to a spectrum of ethanol dosing. For example, the systems may be used to assess the effects of alcohol concentration (i.e., for example dose response evaluations), duration of exposure and/or frequency of exposure. Using this type of modelling flexibility the testing systems can mimic cellular and tissue impacts to moderate, binge and/or heavy alcohol consumption. It is further believed, that specific cell-type contributions to these effects can be more precisely defined by comparing microfluidic systems that have been constructed both with and without human Kupffer cells (KC).

In general it is believed that these microfluidic systems can show the effects of physiological relevant ethanol exposure on hepatocytes using endpoints that are relevant to human pathology. For example, these systems can determine the progression of liver tissue models through the different ASH stages including, but not limited to: (1) alcoholic fatty liver (marked by lipidogenesis, hepatocytes and LSEC apoptosis, and mitochondrial damage), and (2) alcoholic hepatitis (marked by pro-inflammatory signals). Furthermore, the reversibility of the pathology in association to severity and time can be determined as is observed in various phenotypes of the human disease.

In some embodiments, the presently contemplated microphysiological system may be constructed with primary human hepatic cells inside a microengineered environment incorporating fluid flow (e.g., physiologic buffers comprising nutrients and/or test compounds). Although it is not necessary to understand the mechanism of an invention, it is believed that this microengineered environment can emulate the liver sinusoid space architecture and allows for dynamic studies of liver functions over time. Because of these advantages, as compared to the previously described in vitro systems to study ALD, physiologically-relevant ethanol concentrations found in human patients blood may be evaluated to induce the different stages of ALD progression (e.g., fatty liver, steatosis and fibrosis) and demonstrate various points of reversibility.

The microfluidic systems as contemplated herein may provide a platform for new drug development and screening for liver injury therapy and/or protection for alcohol users. In particular, as these test platforms are based on primary hepatic cells, these test cells can be derived from specific patients to provide insights on patient-specific responses. Alternatively, these microfluidic tissue testing systems may be used to support targeted biomarker evaluations and drug discovery efforts that may translate ALD preclinical data into a testable and clinically relevant ALD model enabling to test and characterize drug efficacy and toxicity. For example, common causes of liver injury may include but are not limited to, drug induced liver injury (DILI), alcohol toxicity, obesity, diabetes, infection and/or hepatocellular carcinoma (HCC). The biologically consequence or symptoms of such liver injury may include, but is not limited to, metabolic dysregulation, iron dysregulation (e.g., anemia/iron overload), carbohydrate imbalance, lipid imbalance, late onset diabetes, vitamin storage dysregulation, biliary tract damage, inflammation and/or fibrosis.

In one embodiment, a microfluidic tissue testing system comprises two chambers separated by a porous membrane (pore size ˜7 microns). In one embodiment, a first chamber comprises a plurality of parenchymal cells (e.g., hepatocytes) sandwiched between two layers of extracellular membrane (ECM) proteins (i.e., collagen and fibronectin) on one side of a porous membrane and a second chamber comprises a plurality of non-parenchymal cells attached on the other side of the ECM-coated porous membrane. In one embodiment, the non-parenchymal cells include, but are not limited to, LSECs, HSCs and/or KCs. See, FIG. 1B and FIG. 1C. In one embodiment, the chamber comprises at least two cell types (a co-culture). In one embodiment, the chamber comprises at least three cell types (a tri-culture). In one embodiment, the chamber comprises at least four cell types (a quad-culture). In one embodiment, a tri-culture comprises an ECM molecular coating under the hepatocytes and Matrigel® molecular coating above hepatocytes. See, FIG. 59A. In one embodiment, a tri-culture comprises an ECM molecular coating under the hepatocytes and a 3D gel overlay of less than 100 μm above the hepatocytes. See, FIG. 59B. In one embodiment, a tri-culture comprises a 3D gel underlay under the hepatocytes and a 3D gel overlay above the hepatocytes. See, FIG. 59C. In one embodiment, a quad-culture comprises an ECM molecular coating under the hepatocytes and a Matrigel® molecular coating above hepatocytes. See, FIG. 60A. In one embodiment, a quad-culture comprises an ECM molecular coating under the hepatocytes and a 3D gel overlay of less than 100 μm above the hepatocytes. See, FIG. 60B. In one embodiment, a quad-culture comprises a 3D gel underlay under the hepatocytes and a 3D gel overlay above the hepatocytes. See, FIG. 60C.

In one embodiment, the present invention contemplates a method of using the microfluidic tissue testing system where the two chambers are perfused independently and each has a relative flow rate that enables survival, differentiation and/or maturation of the different cell types. For example, a plurality of primary human hepatocytes (PHHs) attached within the first chamber forms a confluent layer. Furthermore, a co-culture with HSCs, KCs, and LSECs provides an improvement of in vivo-like functionality (for example, bile duct connectivity) and hepatic gene expression (for example, CYP3A4 activity) as compared to a conventional plate-based PHH monolayer. See, FIGS. 1D and 1E, respectively. Hepatocytes that were differentiated and matured in a microfluidic tissue testing system have been observed to release higher levels of albumin and urea than traditional plate studies, demonstrating over time hepatocyte maturation in culture. Although it is not necessary to understand the mechanism of an invention, it is believed that the presently disclosed microfluidic tissue testing system, containing separate cell chambers separated by a porous membrane obviates the need to micropattern ECM on a single surface while allowing co-cultured cells to interact via contact-mediated (membrane processes) and paracrine signaling.

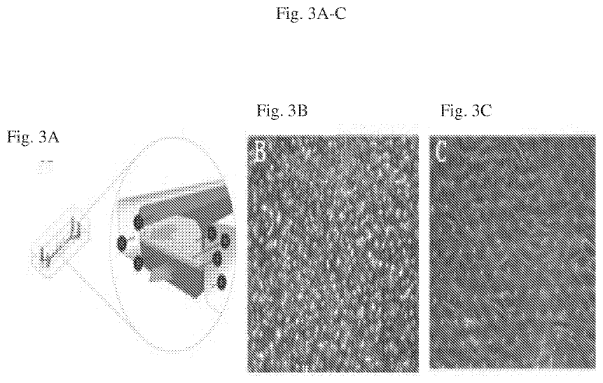

In one embodiment, the present invention contemplates a microfluidic tissue testing system using primary human hepatocytes co-cultured with human primary LSEC to engineer fully differentiated hepatic functions. In summary, the microfluidic tissue testing system contains one hepatocyte full monolayer in the top chamber of a microfluidic channel and one LSEC complete monolayer in the bottom chamber of the microfluidic channel. See, FIG. 3. In some embodiments, the LSEC monolayer is associated with human primary KCs while in other embodiment the human primary KCs are absent. In one embodiment, human primary KCs are present with a physiological density of 1 KC per 10 LSECs.

B. Exposure to Physiologically-Relevant Ethanol Doses

In some embodiments, the microfluidic tissue testing system can be used to evaluate physiologically relevant ethanol exposures mimicking different categories of alcohol consumption including, but not limited to, moderate, binge and heavy drinkers. For example, different ethanol exposure conditions including, but not limited to, concentration, duration and/or frequency can have relevance to these major alcohol consumption categories. Although it is not necessary to understand the mechanism of an invention, it is believed that testing using physiologically relevant ethanol concentrations enable the translation of the in vitro data for clinical application to human patients.

Precise ethanol dosing conditions can be determined using hepatocyte and LSEC viability quantification tests as follows:
i) Hepatotoxicity: Assessed by, for example, viability markers such as albumin and urea release, calcein AM (live cell quantification), Eh-1 (necrosis quantification);
ii) Apoptosis: Assessed by, for example, Caspase 3; and
iii) Mitochondrial membrane potentials: Assessed by, for example, tetramethyl rhodamine methyl ester (TMRM) or safranin.
   These assays result in a table of hepatic cell viability over time of ethanol exposure at different concentrations. For example, ethanol dosing conditions could provide different response profiles for comparison, such as: (1) low and slow toxicity, (2) high and fast toxicity, (3) high and slow toxicity. Although it is not necessary to understand the mechanism of an invention, it is believed that due to the lack of reliable available clinical data on ethanol exposure in moderate, binge and heavy drinkers to determine relevant in vitro physiological concentrations of ethanol, the present invention may result in an adjustment to the generally accepted dosing ranges for the three general categories subsequent to obtaining clear phenotypic differences between ethanol exposure groups.

Physiological concentrations of ethanol (0.5 μL/mL equivalent to a 0.04% blood alcohol concentration (BAC); 1.0 μL/mL equivalent to 0.08% BAC); and 2.0 μL/mL equivalent to 0.16% BAC) were incubated with a tri-culture of hepatocytes and Kupffer cells (36 chips total, n=6 per condition). The cellular response was detected using the cellular parameters of glucose, albumin, cholesterol, intracellular cholesterol, triglycerides, glycogen, ethanol and cytokines. The cellular parameters were measured over a time period of seven (7) days including a dosing days 1-3, followed by recovery days 4-7. See, FIG. 69. Cell morphology of the hepatocyte/Kupffer cell layers were assessed subsequent to cell seeding but before expose to perfusion medium. See, FIGS. 70A and 70B. Cell morphology of the hepatocyte/Kupffer cell layers were assessed subsequent to perfusion medium exposure but before ethanol administration. See, FIGS. 71A and 71B. Cell morphology of the hepatocyte/Kupffer cell layers were assessed after twenty-four hours of exposure to several physiological ethanol concentrations. See FIGS. 72 and 73. Cell morphology of the hepatocyte/Kupffer cell layers were assessed after forty-eight hours of exposure to several physiological ethanol concentrations. See FIG. 74. Cell morphology of the hepatocyte/Kupffer cell layers were assessed forty-eight hours after exposure to several physiological ethanol concentrations was discontinued (e.g., a recovery period). See FIG. 75. Cell morphology of the hepatocyte/Kupffer cell layers were assessed one-hundred and twenty hours after exposure to several physiological ethanol concentrations was discontinued (e.g., a recovery period). See FIG. 76. Lipid droplet accumulation was assessed within the hepatocytes after forty-eight hours of exposure to various physiological ethanol concentrations. See, FIGS. 77A and 77B. Lipid droplet accumulation was assessed within the hepatocytes one hundred and twenty hours after exposure to various physiological ethanol concentrations was discontinued (e.g., a recovery period). See, FIGS. 78A and 78B. Cholesterol accumulation was assessed within the hepatocytes after twenty-four and forty-eight hours of exposure to various physiological ethanol concentrations. See, FIGS. 79A and 79B, respectively. Cholesterol accumulation was assessed within the hepatocytes forty-eight hours and one hundred and twenty hours after exposure to various physiological ethanol concentrations was discontinued (e.g., a recovery period). Lipid accumulations were observed to be statistically significant at both recovery end points. See, FIGS. 80A and 80B, respectively. Cholesterol release was increased under ethanol interaction with the Liver-Chip for 24 h and intensified over 48 h. However after 48 h of recovery the cholesterol levels were back to normal demonstrating the ability of this model to reproduce recovery. Glucose accumulation was assessed within the hepatocytes after twenty-four and forty-eight hours of exposure to various physiological ethanol concentrations. See, FIGS. 81A and 81B, respectively. Glucose accumulation was assessed within the hepatocytes forty-eight hours and one hundred and twenty hours after exposure to various physiological ethanol concentrations was discontinued (e.g., a recovery period). See, FIGS. 82A and 82B, respectively. Triglyceride accumulation was assessed within the hepatocytes after twenty-four and forty-eight hours of exposure to various physiological ethanol concentrations. See, FIGS. 83A and 83B, respectively. Triglyceride accumulation was assessed within the hepatocytes forty-eight hours and one hundred and twenty hours after exposure to various physiological ethanol concentrations was discontinued (e.g., a recovery period). See, FIGS. 84A and 84B, respectively.

Albumin accumulation was assessed within the hepatocytes after twenty-four and forty-eight hours of exposure to various physiological ethanol concentrations. See, FIGS. 85A and 85B, respectively. Albumin accumulation was assessed within the hepatocytes forty-eight hours and one hundred and twenty hours after exposure to various physiological ethanol concentrations was discontinued (e.g., a recovery period). See, FIGS. 86A and 86B, respectively.

Ethanol concentrations were verified by direct measurement after twenty-four hours of perfusion and forty-eight hours of perfusion. See, FIGS. 87A and 87B.

C. Alcoholic Liver Disease Progressive Stage Expression

The data presented herein validates the in vivo relevance of human ASH development using a microfluidic tissue testing system as disclosed herein. For example, the data characterizes disease progression and time-dependent capability for reversibility of the pathology. In one embodiment, the microfluidic tissue testing system characterizes several aspects of ASH developed under ethanol exposure under physiologically relevant ethanol dosing conditions as described above as evaluated by determination of a plurality of ASH biomarkers. For example, ASH biomarkers can be assessed in hepatocytes and LSEC during the early stage of ALD when overt cellular toxicity events during the late stages of ALD responses associated with other cell types are not taking place. Alternatively, ASH phenotypes may be assessed in the presence or absence of KCs, thereby determining KC-dependent ASH phenotypes. Combining these two different data sets can determine different ASH phenotype profiles, for example, KC-dependent and KC-independent cytokine profiles produced in response to different ethanol exposure conditions. In one embodiment, the ALD biomarkers comprise alcoholic fatty liver/steatosis markers. In one embodiment, the ALD biomarkers comprise lipidogenesis markers. In one embodiment, the ALD biomarkers comprise hepatotoxicity markers. In one embodiment, the ALD biomarkers comprise inflammation biomarkers and their proposed genes. See, Tables 2 and 3.

TABLE 2
TASH Biomarkers

| Ethanol Metabolism | | Hepatotoxicity | |
|---|---|---|---|
| and Lipidogenesis | | and Inflammation | |
| Phenotype | Technique | Phenotype | Technique |
| Lipid Droplets | IF | hepatocytes and LSEC | IF an IA |
| | | viability and apoptosis | |
| Quantification of | qPCR | Mitochondrial | IF and IA |
| expression changes | | damage and | |
| in genes related to | | generation of | |
| glucose and lipids | | free radicals | |
| metabolization | | | |
| Cytochrome P450 2E1 | — | Albumin and | qPCR/ELISA |
| (CYP2E1) expression | | Urea release | |
| Quantification of | — | Albumin and | ELISA |
| Free Fat Acid, | | Urea release | |
| Cholesterol and | | | |
| Glucose release | | | |
| CYP activity | — | Inflammasome: | ELISA |
| | | Quantification | |
| | | of activated | |
| | | IL-1β and IL-18 | |
| — | — | Biliary function | — |

TABLE 3
Biomarker Genes Of Interest.

| Pathway of Interest | Genes of Interest |
|---|---|
| Carbohydrate | ACLY, G6PC, G6PD, GCK, GSK3B, |
| Metabolism | MLXIPL, PCK2, PDK4, PKLR, RBP4. |
| Cholesterol | ABCA1, APOA1, APOB, APOC3, APOE, |
| Metabolism/ | CD36, CNBP, CYP2E1, CYP7A1, |
| Transport | HMGCR. LDLR, LEPR, NR1H2, |
| | NR1H3, NR1H4, PPARA, PPARG, |
| | PRKAA1, RXRA, SREBF1, SREBF2 |
| Other Lipid | ACACA, ACSL5, ACSM3, DGAT2, |
| Metabolism/ | FABP3, FABP5, FASN, GK, HNF4A, |
| Transport | LPL, PNPLA3, PPA1, SCD, SC27A5 |
| Alcohol | ADH (family), ALDH (ADH1B, ADH1C, |
| Metabolism-Related | and ALDH2), PNPLA3, TM6SF2, |
| | MBOAT7, CYP2E1 |

Furthermore, the use of these ALD biomarkers can also be used to determine the progression from reversibility to irreversibility of ASH phenotypes and specific liver functions.

These ALD biomarkers are further characterized as follows:

i) Ethanol Metabolism Biomarkers.

CYP2E1 is believed to be one of the enzyme systems that can metabolize alcohol (e.g., ethanol) to AA. CYP2E3 is inducible and can be upregulated 10- to 20-fold in heavy drinkers. Apart from generating AA, CYP2E1 also contributes to oxidative damage by the formation of ROS. In one embodiment, the methods contemplated herein quantify CYP2E1 expression and ROS formation as biomarkers for increased ethanol metabolism.

The data presented herein demonstrates physiologically relevant CYP1A, CYP2B, and CYP3A activities maintained for 2-4 weeks in the presently disclosed hepatocyte microchip as compared to conventional static culture technology. See, FIGS. 37A-C.

Co-culture, tri-culture and quad-culture embodiments of the presently discloses hepatocyte microchip all show higher levels of CYP3A activity than conventional culture. CYP3A4 enzyme activity and gene expression showed in vivo relevant levels after ten (10) days of culture. See, FIGS. 57A-B.

ii) Lipogenesis Biomarkers

Alcoholic fatty liver is believed to be an initial liver lesion during ALD, and fatty liver is the result of lipogenesis. Increased lipogenesis can be quantified using lipid droplet accumulation inside hepatocytes and/or the release of fatty acid, triglycerides and cholesterol. In one embodiment, the methods contemplated herein determine the time and dose correlation between ethanol dosing and increased lipogenesis and ROS formation. Other lipogenic markers encompass steatosis biomarkers including, but not limited to, metabolic dysfunction (resulting in lipid accumulation); inflammation, mitochondria dysfunction, apoptosis and/or bile.

iii) Inflammation Biomarkers

Inflammation can occur in ASH and is believed to lead to more severe aspects of ALD including, but not limited to fibrosis or cirrhosis. Pro-inflammatory compounds well known in the art may be assessed in the microfluidic tissue testing system that are constructed with and/or without KCs. For example, cytokine release profile may be evaluated over time under different ethanol exposure conditions (supra). These results can determine inflammation states associated with ALD start points and/or progressive mechanisms of tissue toxicity in an in vitro model. This data can also be used to assess reversibility strategies that facilitates the development of new drugs.

iv) Biliary Function Biomarkers

Bile duct canaliculi formation is believed to be a hepatic function marker. Calcein-AM may be provided to hepatic tissue where it is converted to a green-fluorescent calcein after ester hydrolysis by intracellular esterases. Calcein transport is mediated by MRP2, which is expressed in the canalicular part of hepatocytes. In one embodiment, the present invention contemplates a method for evaluation dysregulation of biliary function by quantifying bile canaliculi structures (Calcein-AM stain) before and after ethanol exposure.

Bile caniculi formation after three (3) days of culture in the presently disclosed hepatocyte microchip has been verified using transporter-mediated CDFDA efflux. See, FIGS. 43A-B. MRP2 is expressed in the bile canalicular (apical) part of the hepatocytes and functions in biliary transport. MPR2 expression has been observed after fourteen (14) days of hepatocyte cell culturing. Notably, the presently disclosed hepatocyte microchip culture results in improved transported expression and localization as compared to a conventional sandwich plated culture. See, FIGS. 44A-B.

v) ASH Reversibility Point Biomarkers

While literature on ALD demonstrated that steatosis and inflammation are usually reversible upon ethanol abstinence, these effects can be verified using a human-relevant microfluidic tissue testing system, as abstinence is the only well-established treatment for ALD. Using an advantage that the presently disclosed liver microphysiological system can sustain long-term hepatic viability and function, the present method contemplates evaluating changes in ASH markers during an alcoholic recovery period in which the ethanol exposure is removed. Although it is not necessary to understand the mechanism of an invention, it is believed that a microfluidic tissue testing system can enable the identification of reversibility and irreversibility points for ASH phenotypes under different ethanol conditions.

vi) Albumin Biomarkers

The data presented herein demonstrate that albumin release over twenty-eight (28) days is increased in hepatocytes cultured in the presently disclosed microchip as compared to conventional static cell culture plates. See, FIG. 41A

vii) Urea Biomarkers

The data presented herein demonstrate that urea release over twenty-eight (28) days is increased in hepatocytes cultured in the presently disclosed microchip as compared to conventional static cell culture plates. See, FIG. 41B.

D. NALD Lipid Accumulation

In one embodiment, the present invention contemplates a microfluidic device or chip comprising a liver tissue (e.g. a population of hepatic cells, including in one embodiment a variety of hepatic cell types) exhibiting a non-alcohol-induced steatosis phenotype (e.g., lipid accumulation). In one embodiment, a progressive non-alcohol-induced steatosis phenotype is induced by a toxic compound. In one embodiment, the present invention contemplates a method comprising exposing the liver tissue (having the phenotype) to at least one test compound. In one embodiment, the test compound slows steatosis progression. In one embodiment, the test compound stops steatosis progression. In one embodiment, the test compound reverses steatosis progression.

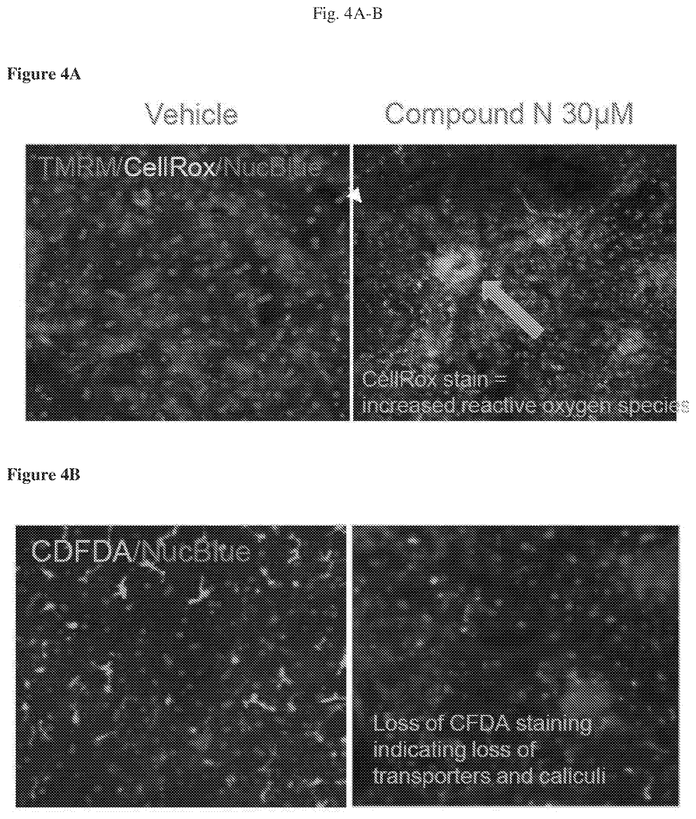

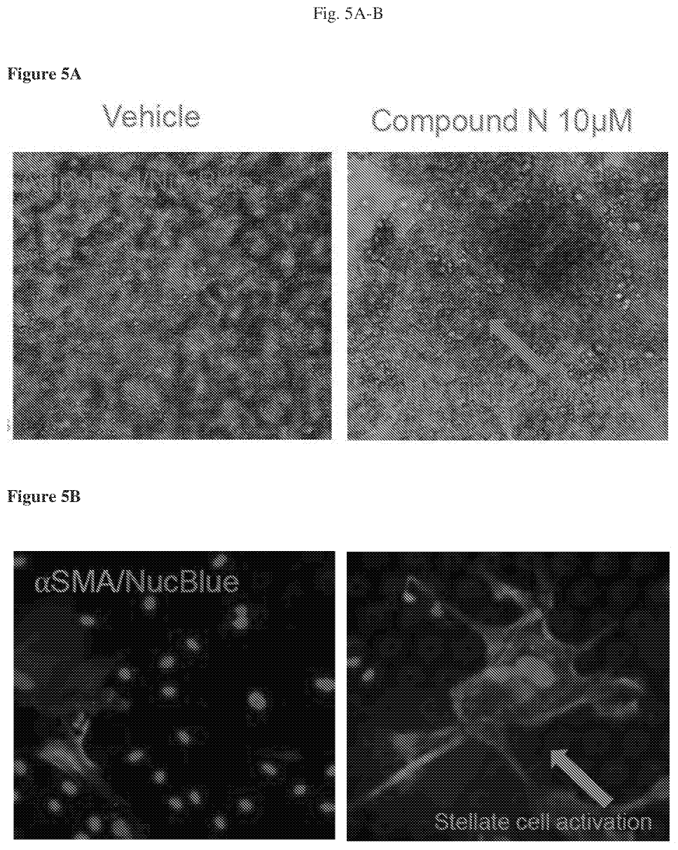

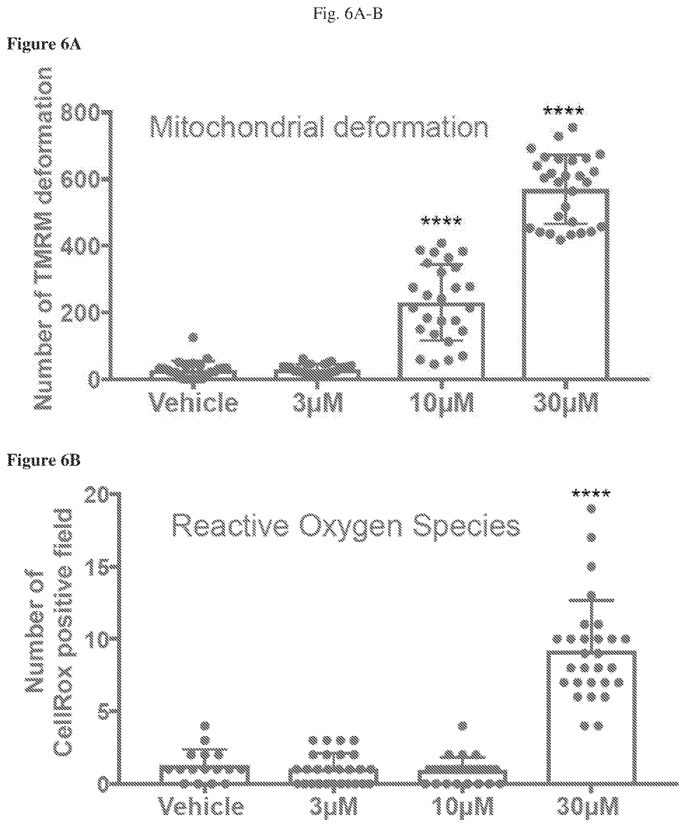

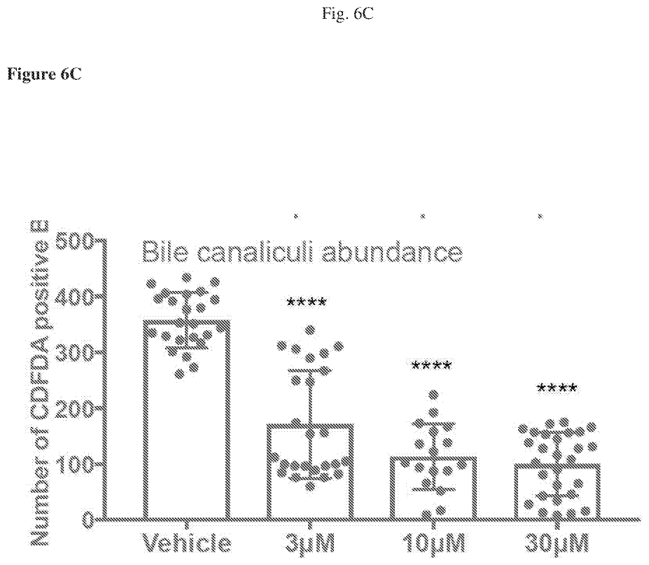

The data shown herein demonstrates the imaging of lipid accumulation in hepatocytes using Nile Red. As lipid accumulation is a marker of liver disease, the presently disclosed hepatocyte microchip technology was used to assess steatosis progress in a non-alcoholic liver disease model. For example, lipid accumulation was intentionally induced by a toxic compound using a hepatocyte microchip to create a steatosis liver disease model. The CPD-N treatment was observed to: i) deform the mitochondrial membrane potential marker (TMRM (red)) and increase hepatic oxidative stress as measured by CellRox dye (blue) (FIG. 4A); ii) decrease bile canaliculi structures as measured by (5, 6)-carboxy-2′,7′-dichlorofluorescein diacetate (carboxy-DCFDA) dye (green) (FIG. 4B); iii) increase steatosis as measured by an AdipoRed dye (red) FIG. 5A; and v) activate stellate cells as measured by aSMA expression (FIG. 5B). All assessments were made in the presence of NucBlue to monitor chromosomal integrity. A dose-dependent relationship was observed for mitochondrial deformation, reactive oxygen species production and bile canaliculi abundance. FIGS. 6A, 6B and 6C, respectively.

The data were produced in comparison of a drug that failed during Phase III clinical trials. Although it is not necessary to understand the mechanism of an invention, it is believed that the presently disclosed hepatocyte microchip technology surprisingly predicted drug failure due to toxicity that is not possible with currently available animal models or other in vitro testing model systems. Furthermore, it is also believed that hepatocyte microchip technology is able to measure multiple functional outputs providing a richness of the data set to enable a new understanding of the complex biology driving the observed toxicity. In one embodiment, the hepatocyte microchip technology can determine differences in the response of hepatic tissues in vitro (e.g. a population of hepatic cells, including in one embodiment a variety of hepatic cell types) from two or more different genetic predispositions (e.g., inter-patient variation). In other embodiments, the disease model could be used for drug discovery and the testing of drug efficacy (e.g. for drugs that treat steatosis or for drugs that prevent the steatosis from progressing to cirrhosis), for studying the mechanisms of the disease and to discover new therapeutics.

In one embodiment, the present invention contemplates a microfluidic device or chip comprising a liver tissue (e.g. a population of hepatic cells, including in one embodiment a variety of hepatic cell types) and a method for inducing non-alcohol-induced steatosis phenotype (e.g., lipid accumulation). In one embodiment, the present invention contemplates exposing the hepatocyte microchip tissue in vitro to a high concentration at least one lipid or fatty acid (e.g., for example, oleic acid in a culture media). The exposure results in the phenotype, providing an in vitro model of the disease. The model can be used to better understand the disease and as a platform for drug testing and drug discovery. In one embodiment, the method further comprises exposing the hepatocyte microchip tissue (having the phenotype) to at least one test compound. In one embodiment, the test compound slows steatosis progression. In one embodiment, the test compound stops steatosis progression. In one embodiment, the test compound reverses steatosis progression.

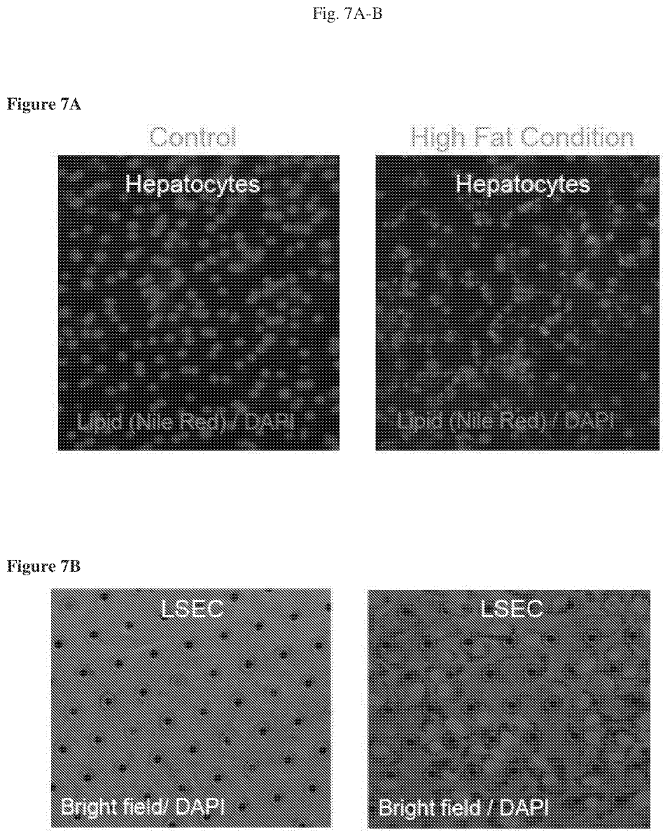

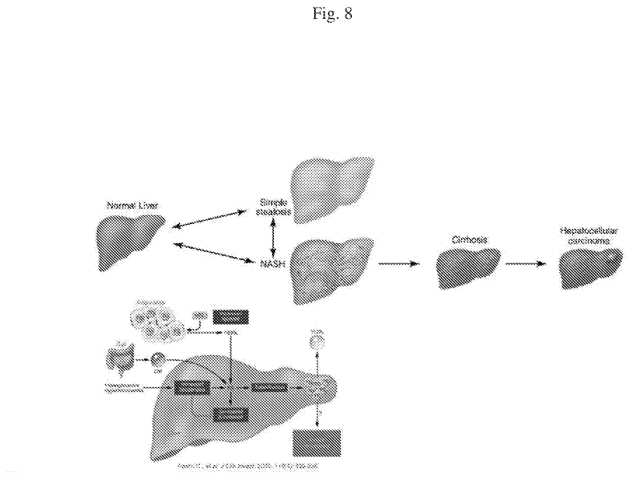

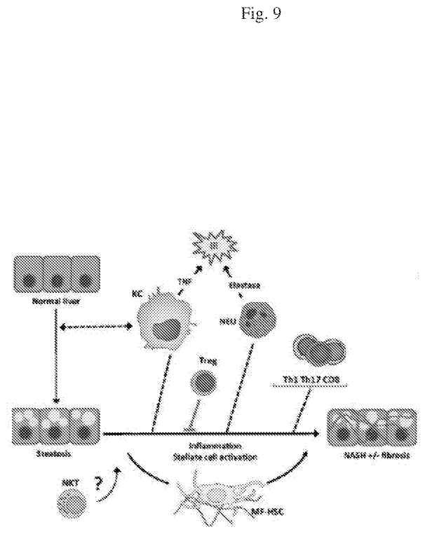

The data shown herein demonstrates that exposing hepatocytes on a hepatocyte microchip to fatty acid results in lipid accumulation as seen by a high concentration of lipid droplets. FIG. 7A. LSEC cells exposed to a high lipid or fatty acid media demonstrated morphological parameters indicative of injury/stress exposure. FIG. 7B. These data show that hepatic tissue on a hepatocyte microchip platform responds to a high lipid environment which provides a reliable in vitro model to characterize lipid homeostasis dysregulation.

E. NALD Inflammation

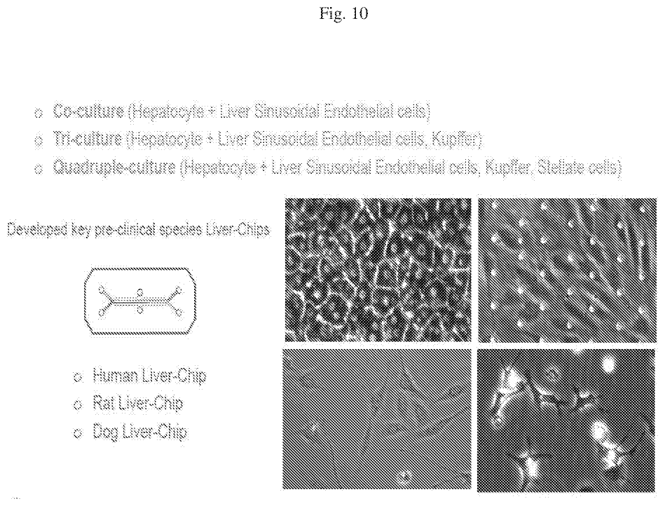

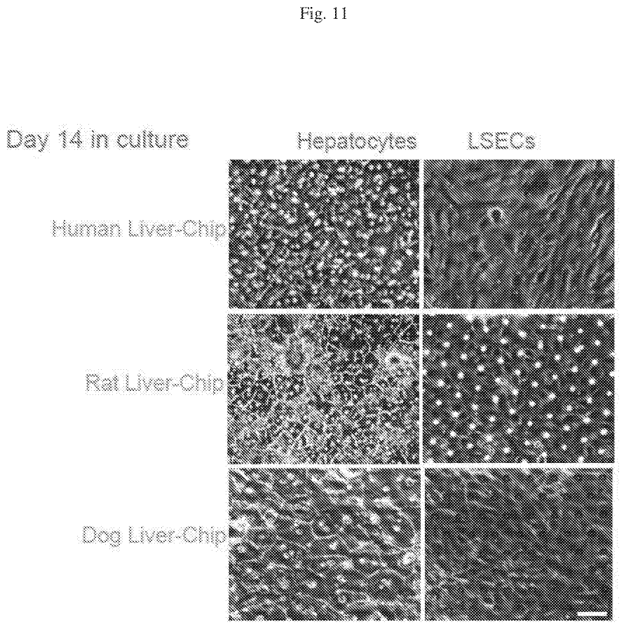

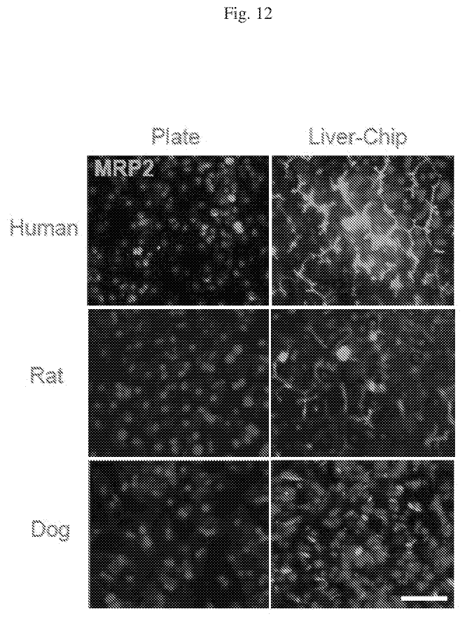

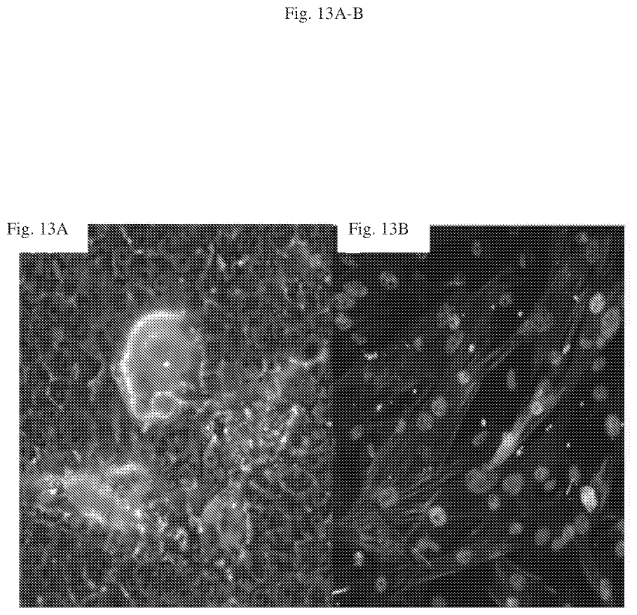

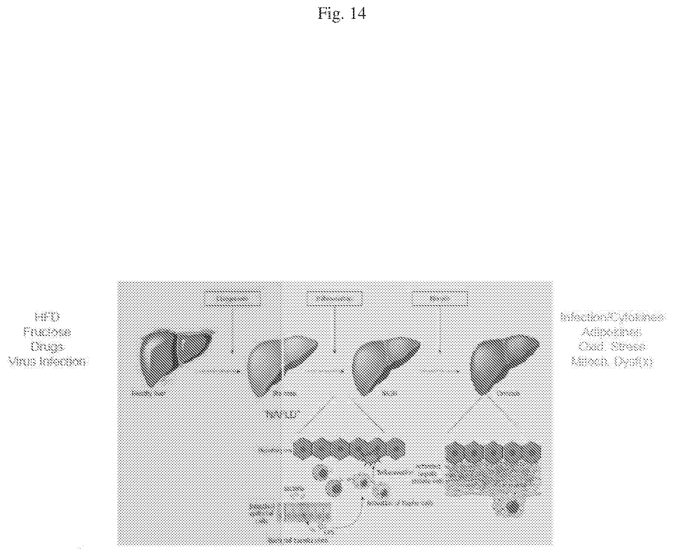

In one embodiment, the present invention contemplates a microfluidic device or chip comprising liver tissue (e.g. a population of hepatic cells in vitro, including in one embodiment a variety of hepatic cell types) exhibiting an inflammatory non-alcohol-induced steatosis phenotype. FIG. 10. In one embodiment, the inflammatory non-alcohol-induced steatosis phenotype is induced by a compound. In one embodiment, the inflammatory non-alcohol-induced steatosis is induced by exposure to a concentration of a fatty acid. In one embodiment, the method further comprises co-culturing the hepatic tissue with immune cells (e.g. lymphocytes, macrophages, etc.). In one embodiment, the co-culturing enhances the inflammatory non-alcoholic-induced steatosis phenotype.

As shown herein, the hepatocyte microchip platform allows co-culturing of hepatocytes with a plurality of cells, including, but not limited to, immune cells, LSEC cells, Kupffer cells and/or stellate cells. FIG. 10. In one embodiment, the present invention contemplates a hepatocyte microchip comprising at least one hepatic stellate cell. Although it is not necessary to understand the mechanism of an invention, it is believed that the three dimensional (3D) topography of the microchip channels provide a superior environment for stellate cell growth and differentiation as compared to a conventional two dimensional (2D) plate cell culture technology. It can be seen that while stellate cells present an activated morphology when culture in 2D collagen coated plates, the stellate cells present an in vivo physiological star shape morphology when cultured in a 3D topography provided by a microchip microchannel. See, FIG. 58A-B.

### VI. Cell Culture Media

A. Endothelial Cell Culture Media

In one embodiment, the present invention contemplates a hepatocyte microchip culture system comprising an endothelial cell culture media, wherein the media comprises at least one component including, but not limited to, CSC (e.g., 2-10%), DMEM, glucose, GlutaMax®, NEAA, FBS (e.g., 5%), ITS GIBCO° or PromoCell® complements. In one embodiment, the media may further comprise EGM-2 or ECGM-2. See, Table TBD.

TABLE 4
Exemplary components of EGM-2 And ECGM-2.

| Final supplement concentrations | Endothelial Cell | | Endothelial Cell | |
|---|---|---|---|---|
| (after addition to the medium) | Growth Medium | | Growth Medium 2 | |
| Fetal Calf Serum | 0.02 | ml/ml | 0.02 | ml/ml |
| Endothelial Cell Growth Supplement | 0.004 | ml/ml | — | |
| Epidermal Growth Factor | 0.1 | ng/ml | 5 | ng/ml |
| (recombinant human) | | | | |
| Basic Fibroblast Growth Factor | 1 | ng/ml | 10 | ng/ml |
| (recombinant human) | | | | |
| Insulin-like Growth | — | | 20 | ng/ml |
| Factor (Long R3 IGF) | | | | |
| Vascular Endothelial | — | | 0.5 | ng/ml |
| Growth Factor 165 | | | | |
| (recombinant human) | | | | |
| Ascorbic Acid | — | | 1 | μg/ml |
| Heparin | 90 | μg/ml | 22.5 | μg/ml |
| Hydrocortisone | 1 | μg/ml | 0.2 | μg/ml |

Endothelial cells, such as LSECs, can be used to test viability in different media by imaging Ac LDL uptake. See, FIG. 55. Several media showed equivalent confluency of a co-culture of LSECs (2% CSC; AdDMEM; and AdDMEM:WEM (1:1)) and hepatocytes (2% WEM) of seven (7) days of culturing. See, FIG. 56. LSECs were further evaluated in the following media:
1. CSC 2%
2. Advanced DMEM/F12 GlutaMax, complete with EGM-2 1% Serum
3. Advanced DMEM/F12 GlutaMax (1:1400), complete with EGM-2 1% Serum
4. (1:1) Advanced DMEM/F12: WEM GlutaMax, complete with EGM-2 1% Serum
5. (1:1) Advanced DMEM/F12: WEM GlutaMax (1:1400), complete with EGM-2 1% Serum
6. DMEM medium glucose (2 g/L), NEAA (1:200), GlutaMax (1:1400), ITS Corning with linoic acid (concentration), +EGM-2+2% Serum.
7. DMEM low glucose (1 g/L), NEAA (1:200), GlutaMax (1:1400), ITS Corning with linoic acid (1:100)), +EGM-2+2% Serum.
8. DMEM low glucose (1 g/L), NEAA (1:200), GlutaMax (1:1400), ITS Corning with linoic acid (1:200), +EGM-2+2% Serum.
9. DMEM low glucose (1 g/L), NEAA (1:200), GlutaMax (1:1400), ITS Corning with linoic acid (1:400), +EGM-2+2% Serum.

The data demonstrate cell viability using each media. See, FIG. 52. Other media compositions are also contemplated including, but not limited to, i) DMEM low glucose (1 g/L), NEAA (1:200), GlutaMax® (1:1400), +1% Serum; or ii) DMEM low glucose (1 g/L), NEAA (1:200), GlutaMax® (1:1400), ITS Corning® with linoic acid (1:400), +EGM-2 or PromoCell® complements.

Preliminary testing provided the following media conditions that resulted in viable endothelial cells in a microchip as presently disclosed:
1. Advanced DMEM/F12 2%+GlutaMax®+EGM
2. Advanced DMEM/F12+GlutaMax®+WEM+EGM+2% FBS
3. glucose (e.g., between approximately 3.7 g/L to 1 g/L)
4. NEAA (Non-Essential Amino Acid)
5. Low glutamine (e.g., GlutaMax® 35.7 μl in 50 mL)
6. No Sodium Pyruvate
7. Dialyzed serum
8. Charcon filter
9. No Serum
10. Cholesterol
11. VEGF (fibroblast growth factor)
12. IGF (Insulin-like growth factor 1)
13. FGF (fibroblast growth factor)
14. Hydrocortisone
15. EGF (Epidermal growth factor)

In one embodiment, the present invention contemplates an endothelial cell media comprising advanced DMEM/F12 GlutaMax® with complete EGM-2 1% serum. In one embodiment, the present invention contemplates an endothelial cell media comprising DMEM (high glucose), NEAA (1:200), GlutaMax® (1:100), ITS Corning® with linoic acid, AlbuMax®, EGM-2 or PromoCell® complements and 1% serum. In one embodiment, the present invention contemplates an endothelial cell media comprising DMEM (low glucose), NEAA (1:200), GlutaMax® (1:1400), ITS Corning® with linoic acid, AlbuMax®, EGM-2 or PromoCell® complements +1% serum.

B. Hepatocyte Cell Culture Media

In one embodiment, the present invention contemplates a hepatocyte microchip culture system comprising a hepatocyte cell culture media, wherein the media comprises at least one component including, but not limited to insulin (e.g., ITS GIBCO®; insulin/transferrin); glucose (e.g., 1-2 g/L WEM or DMEM); dexamethasone (e.g., 1 ul in 50 ml), glutamine (e.g., GlutaMAX®); fatty acids and/or amino acids. In some embodiment, DMEM media comprises the following components:

TABLE 5
Hepatocyte Cell Culture Media.

| Advanced DMEM/F12 (12634) | | | | Advanced DMEM (12491) | | | |
|---|---|---|---|---|---|---|---|
| Category | Component | mg/L | OBS | Category | Component | mg/L | OBS |
| — | L-Cysteine | 17.56 | — | — | — | — | — |
| | hydrocrilorlde-H20 | | | | | | |
| Vitamins | Ascorbic acid | 2.5 | — | Vitamins | Ascorbic acid | 2.5 | — |
| | phosphate | | | | phosphate | | |
| — | Biotin | 0.0035 | — | — | — | — | — |
| — | Choline Chloride | 898 | — | — | — | — | — |
| | (bigger concentration) | | | | | | |
| — | Vitamin B12 | 0.68 | — | — | — | — | — |
| — | i-inositol | 12.6 | — | — | — | — | — |
| — | Cupric Sulfate | 0.0013 | — | Trace | Cupric Sulfate | 0.00125 | — |
| | | | | elements | | | |
| — | Ferric Sulfate | 0.417 | — | — | — | — | — |
| — | Magnesium Chloride | 28.64 | — | — | — | — | — |
| | (anhydrous) | | | | | | |
| Inorganic | Sodium Phosphate | 71.02 | — | Inorganic | Sodium Phosphate | 125 | monobasic |
| salts | dibasic anhydrous | | | salts | dibasic anhydrous | | in DMEM |
| — | Zinc sulfate | 0.864 | — | — | — | — | — |
| | Albumax II | 400 | | Proteins | Albumax II | 400 | |
| Proteins | | | | | | | |
| Proteins | Human Transferrin | 7.5 | ITS (5 | | Human | 7.5 | ITS (5 |
| | | | mg/L) | Proteins | Transferring | | mg/L) |
| | | | | | (Holo) | | |
| Proteins | Insulin Recombinant | 10 | ITS | Proteins | Insulin | 10 | ITS |
| | full chain | | | | Recombinant full | | |
| | | | | | chain | | |
| — | Glutathione, | 1 | — | — | Glutathione | 1 | — |
| | monosodium | | | | (reduced) | | |
| Trace | Ammonium | 3.00 E−04 | — | Trace | Ammonium | 3.00 E−04 | — |
| elements | metavanadate | | | elements | metavanadat | | |
| Trace | Manganous Chloride | 5.00 E−05 | — | Trace | Manganous | 5.00E−05 | — |
| elements | | | | elements | Chloride | | |
| Trace | Sodium Selenite | 0.005 | ITS | Trace | Sodium Selenite | 3.005 | ITS |
| elements | | | (0.0067 | elements | | | (0.0067mg/ |
| | | | mg/L) | | | | L) |
| Other | Ethanolamine | 1.9 | | Other | Ethanolamine | 1.90E+00 | |
| — | Hypoxanthine Na | 2.39 | — | — | — | — | — |
| — | Linoleic Acid | 0.042 | — | — | — | — | F12 has |
| | | | | | | | Glutathione, |
| | | | | | | | monosodium |
| — | Lipoic Acid | 0.105 | — | — | — | — | — |
| — | Putrescine 2HCL | 0.081 | — | — | — | — | — |
| — | Thymidine | 0.365 | — | — | — | — | — |
| — | — | — | — | — | — | — | — |
| — | D-GLUCOSE | 3151 | | | D-GLUCOSE | 4500 | −4.5x |
| | | | | | | | higher than |
| | | | | | | | DMEM |
| — | Sodium pyrovate | 110 | | | Sodium pyruvate | 1.00 E−02 | |
| — | GLUMAMINE | NO | | | GLUMAMINE | — | — |
| — | — | — | — | — | — | — | — |
| — | L-Alanine | 4.45 | 2x less | — | L-Alanine | — | NEAA |
| | | | than | | | | |
| | | | NEAA | | | | |
| — | L-Asparagine | 7.5 | 2x less | — | L-Asparagine | — | NEAA |
| | | | than | | | | |
| | | | NEAA | | | | |
| — | L-Aspartic acid | 6.65 | 2x less | — | L-Aspartic acid | — | NEAA |
| | | | than | | | | |
| | | | NEAA | | | | |
| | L-Glutamic | 7.35 | 2x less | — | L-Glutamic | — | NEAA |
| | acid | | than | | acid | | |
| | | | NEAA | | | | |
| — | L-Proline | 17.25 | 2x less | — | L-Proline | — | NEAA |
| | | | than | | | | |
| | | | NEAA | | | | |
| — | L-Serine | 26.25 | 2x less | — | L-Serine | — | 5x more |
| | | | than | | | | than |
| | | | NEAA | | | | NEAA |

Preliminary testing provided the following media conditions that resulted in viable hepatocyte cells in a microchip as presently disclosed:
1. low glucose (e.g., between approximately 0.2-2 g/L)
2. No sodium pyruvate
3. NEAA
4. Low insulin/transferrin (e.g., ITS GIBCO® between approximately 2.5-5 μl in 50 mL
   a. human recombinant insulin
   b. human transferrin (12.5 mg each)
   c. selenous acid (12.5 μg)
   d. BSA (2.5 g)
   e. linoleic acid (10.7 mg)
5. No BSA or linoleic acid.
6. Low glutamine
   a. GlutaMax® 35.7 μl in 50 mL
   b. Media with L-glutamine at 0.292 g/L
   c. GlutaMAX® (35.7 μl in 50 mL) with 200 mM L-alanyl-L-glutamine dipeptide
7. Human supplements: 14 g/day
8. Low/No dexamethasone (e.g., approximately 0-0.1 μM)
9. Dialyzed serum (0-2%), optionally charcoal-free.

### Experimental

### Example I

### Experimental Setup For Co-Cultured Hepatocyte Microchips

Hepatocyte microchips as contemplated herein can be configured as a co-culture (2 cell types), a tri-culture (3 cell types) or a quad-culture (3 cell types). The hepatocyte microchips comprise a membrane positioned through the center of a microchannel that divides the microchannel into a top channel and a bottom channel.

The membrane surface that is exposed to the top channel in the co-culture, tri-culture and quad-culture embodiment are layered with hepatocyte cells. In the co-culture embodiment the membrane surface that is exposed to the bottom channel is layered with endothelial cells. In the tri-culture embodiment, the membrane surface that is exposed to the bottom channel is layered with endothelial cells and Kupffer cells or hepatic stellate cells. In the quad-culture embodiment, the membrane surface that is exposed to the bottom channel is layered with endothelial cells, Kupffer cells and hepatic stellate cells.

The cell culture media is typically delivered at a rate of 30 μl/hour and may be the same or different between the top channel and bottom channel, for example:

| | | | Bottom Channel | |
|---|---|---|---|---|
| | Top Channel | | (hLSECs or hLSECs + | |
| — | (hHepatocytes) | — | Stellate Cells) | |
| Condition # | Basal Media | % FBS | Basal Media | % FBS |
| 1 | WEM-Complete | 1% | New Media* | 1% |
| 2 | WEM-Complete | 0 % | New Media* | 0% |
| 3 | WEM−/+ | 0% | DMEM/F12 + | 0% |
| | Cholesterol | | Cholesterol (1:1000) | |
| | (1:1000) | | | |
| 4 | WEM Complete | 0% | New Media* but | 0.5% |
| | | | without hFGF-B | |
| | | | and R3-IGF-1 | |

*New Media: (1:1) of adv DMEM/F12:WEM- + Glutamax -> add EGM-2MV 0.5X, 1% FBS.

A cell culture protocol typically lasts for approximately twenty-one days. See, FIG. 28. Hepatocyte cells are seeded on the top membrane surface on Day 0, and the endothelial cells are seeded on Day 2 where both the top channel and bottom channel contain a culture media comprising 2% WEM and 2% CSC. On day 3 the media composition is changed to one of the following conditions:

TABLE 6
Medium Conditions In Channels.

| Condition | Top Channel | Bottom Channel |
|---|---|---|
| 1 | 1% WEM | Ad.DMEM/F12:WEM1% |
| 2 | 0% WEM | Ad.DMEM/F12:WEM 0% |
| 3 | WEM-0% + | Ad.DMEM/F120% + |
| | Cholesterol (1:1000) | Cholesterol (1:1000) |

Alternatively, the media may be changed to have the following conditions:

TABLE 7
Alternative Medium Conditions I.
Media Condition

| Con- | | | % |
|---|---|---|---|
| dition # | Basal Media | EGM-MV2 | FBS |
| 1 | WEM:Adv DMEM/F12 = 1:1 | 0.5X | 1 |
| 2 | WEM:Adv DMEM/F12 = 1:1 | 0.5X | 0.5 |
| 3 | WEM:Adv DMEM/F12 = 1:1 | 0.5X | 0 |
| 4 | WEM:Adv DMEM/F12 = 1:1 | 0.25X | 0.5 |
| 5 | WEM:Adv DMEM/F12 = 1:1 | 0.5X (without hFGF-B) | 0.5 |
| 6 | WEM:Adv DMEM/F12 = 1:1 | 0.5X (without hFGF-B | 0.5 |
| | | and R3-IGF-1) | |
| 7 | Adv DMEM/F12 | 0.5X (without hFGF-B | 0.5 |
| | | and R3-IGF-1) | |

Alternatively, the media may be changed to have the following conditions:

TABLE 8
Alternative Medium Conditions II.

| Condition # | Media Composition | % FBS |
|---|---|---|
| 0 | Adv. DMEM/F12 w/0.5 EGM2: WEM- | 1%-changed |
| | with Cholesterol 1:1000 | to 0% |
| 1 | Adv. DMEM/F12 w/0.5 EGM2: WEM- | 1% |
| 2 | Adv. DMEM/F12 w/0.5 EGM2: WEM- | 2% |
| 3 | Adv. DMEM/F12 w/0.5 EGM2: WEM- | 5% |
| 4 | Rat Endo media | 2% |
| 5 | Rat Endo media | 1% |
| 6 | Rat Endo media | 0% |
| 7 | Endo media with Cholesterol 1:1000 | 0% |
| 8 | Endo: WEM-Complete | 2% |
| 9 | Endo: WEM-Complete | 1% |
| 10 | Endo: WEM-Complete | 0% |
| 11 | Endo: WEM-Complete with | 0% |
| | Cholesterol 1:1000 | |

After seven (7) days of incubation the endothelial cells were stained and imaged for morphology. It was observed that LSECs maintained viability in a variety of media types. See, FIG. 30. These data suggested that LSECs are optimally seeded in a media comprising 10% FBS. The media is best shifted to 2% FBS (fetal bovine serum) until the LSECs are grown to confluence. Further maintenance media is best delivered at 0% FBS.

Optimal ECM conditions were found to be:
R—Rat Tail Collagen I (125 μg/ml )+Fibronectin (125 μg/ml ) in cold DPBS (Control, original ECM) n=12
H—Rat Tail Collagen I (100 μg/ml )+Fibronectin (25 μg/ml ) in cold DPBS
C—Rat Tail Collagen I (100 μg/ml )+Collagen IV (25 μg/ml ) in cold DPBS

Optimal ECM overlay was found to be:
MO_Matrigel −R=9, H=6, C=3
CO—Collagen R=3 H=3

Optimal media conditions were found to be:
Top Channel: WEM—Complete with:
   1—10% FBS->5% FBS
   2—10% FBS->5% FBS->2%FBS
   3—10% FBS->5% FBS->2%FBS->0% FBS
   4—10% FBS->5% FBS->1%FBS
Bottom Channel: Transition from 10% FBS (seeding) to 2% FBS (confluence) to 0% FBS (maintenance).
The data collected from this experimental design demonstrates that
   i) a collagen overlay provides predictable cell growth. See, FIG. 29.
   ii) after six days in culture with a 10% WEM/2% FBS media rat endothelial cells (25 μg/ml or 100 μg/ml overlay) with a collagen coating (0.5 mg/m/) showed a viability that is equivalent to conventional cell culture techniques. See, FIG. 31.

### Example II

### Toxic Compound-Induced Steatosis Treatment Using a Hepatocyte Microchip

This example describes the production and treatment of steatosis on an in vitro hepatocyte microchip using a toxic compound. Hepatic tissue (e.g. a population of hepatic cells in vitro, including in one embodiment a variety of hepatic cell types) was established in vitro in a hepatocyte microchip and contacted with various doses (e.g., 3, 10 and 30 μM) of a toxic compound (e.g., CPD-N) to induce hepatic cell toxicity. Toxicity parameters of these damaged tissues were measured with: i) mitochondrial membrane potential marker (TRMR) to assess mitochondrial toxicity; ii) CellRox to assess the production of oxidative stress; iii) AdipoRed to assess the progression of steatosis; and iv) (5, 6)-carboxy-2′,7′-dichlorofluorescein diacetate (carboxy-CDFDA) to assess expression of alpha smooth muscle actin (□SMA) to determine the presence of bile canaliculi.

### Example III

### Fatty Acid-Induced Steatosis in a Low Glucose/Low Insulin Media

This example describes the production of a steatosis phenotype (allowing for subsequent testing, e.g. drug testing) on an in vitro hepatocyte microchip using a high fatty acid-containing media. The hepatocyte microchip has a microchannel with a membrane creating a top channel and a bottom channel wherein each can grow different cells and/or be exposed to different media.

Hepatic tissue (e.g. a population of hepatic cells in vitro, including in one embodiment a variety of hepatic cell types) was established in a hepatocyte microchip and contacted with a media comprising a high concentration of oleic acid for several days to induce the phenotype (e.g., lipid accumulation and/or an injury/stress morphology). Various parameters of these damaged tissues were measured with: i) Nile Red to assess the progression of steatosis; and iv) brightfield microscopy to assess the induced morphology.

The hepatocyte microchip co-cultures are incubated in a media comprising DMEM, 1 g/L glucose and 8.6 nM insulin. The experimental design included the following groups:

Control: No oleic acid/No ethanol (3 chips)

High Fat: Oleic acid top channel and bottom channel (3 chips)

High Fat bottom: Oleic acid bottom channel only (2 chips)

High Fat High Sugar: With oleic acid and high/glucose (2.5 g/L glucose)

High Sugar: No Oleic Acid and high/glucose (2.5 g/L glucose)

Ethanol: 80 μl/ml ethanol in top channel and bottom channel (2 chips) Apical gut cells may be cultured to evaluate gut connectivity parameters. Lipopolysaccharide (LPS) can optionally be added to any of the above groups to evaluate gut permeability.

The hepatocyte microchip co-cultures are assessed for steatosis biomarkers at Days 5, 7, 8, 9 and 14 during the incubation period that include, but are not limited to: i) morphological changes as assessed by live microscopy; ii) free fatty acid quantification; iii) cholesterol quantification; iv) real-time glucose quantification; v) glycolysis as determined by extracellular acidification; vi) apoptosis and/or necrosis as assessed by live microscopy; vii) cytokine determination (e.g., IL-6 , TNF alpha); and viii) mitochondria function as assessed by live microscopy. Additionally, lipid droplets are assessed using microscopy and a steatosis gene expression pane is generated by quantitative polymerase chain reaction (qPCR: LYSETE®) between approximately 8-64 hours of incubation.

### Example IV

### Fatty Acid-Induced Steatosis in a WEN/CSC Media

This example was performed on a hepatocyte microchip co-culture system (e.g., Liver-Chip). As a control condition, normal cell culture media “diet” (WEN media) was flowed through the top channel of the microchip, while a CSC 10% SFB was flowed through the bottom channel of the microchip. For the experimental condition, a fat “diet” of 1 μM Oleic acid was added to both the top channel WEN media and the 10% CSC bottom channel media. The following measurements were taken: i) Lipid drop staining with Nile Red®; ii) cholesterol quantification; iii) free fatty acid quantification; and iv) glucose quantification.

The data show a progressive accumulation of hepatocyte lipid droplet accumulation over time (e.g., 0, 40 and 64 hours of high fat diet exposure). See, FIGS. 57A-C. Concomitantly, the LSECs showed alterations in morphology as a result of the high fat diet exposure. See, FIG. 7B. Hepatocyte free fatty acids were shown to increase after forty (4) hours of high fat diet exposure. See, FIG. 59.

### Example V

### Fatty Acid-Induced Steatosis and Immune Cells on A Hepatocyte Microchip

This example describes the production of an enhanced steatosis phenotype (allowing for subsequent testing, e.g. drug testing) on an in vitro hepatocyte microchip using a high fatty acid-containing media.

Hepatic tissue (e.g. a population of hepatic cells in vitro, including in one embodiment a variety of hepatic cell types) was established in a hepatocyte microchip and contacted with a media comprising a high concentration of oleic acid for two days to induce the phenotype (e.g., lipid accumulation and/or an injury/stress morphology). Thereafter, immune cells can be added to the model so as to generate an enhanced (inflammation) phenotype, which may provide a better in vitro NASH model.

### Example VI

### Fatty Acid-Induced Steatosis During a Feeding/Fasting Cycle

This example is performed on a hepatocyte microchip co-culture system (e.g., Liver-Chip). The following media conditions will be used: i) Control: No: Oleic Acid; ii) High Fat: iii) fasting/feeding High Fat; and iv) fasting/feeding Control.

### Example VII

### Fatty Acid-Induced Fibrosis During a High Fat Diet

This example is performed on a hepatocyte microchip co-culture system (e.g., Liver-Chip). The following media conditions will be used: i) Control: No: Oleic Acid; ii) High Fat (Oleic Acid).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1-13. (canceled)

14. A method, comprising:
a) providing:
i) a microfluidic device comprising a solid substrate, said solid substrate comprising a membrane, one or more microfluidic channels and hepatic cells;
ii) a physiological buffer solution comprising a physiologically relevant concentration of ethanol;
b) contacting said hepatic cells with said physiological buffer solution under conditions that induces an indicator of a disease phenotype at least one stage of alcoholic liver disease in said hepatic cells; and
c) detecting said at least one indicator of a disease phenotype alcoholic liver disease biomarker in said hepatic tissue.

15. The method of claim 14, wherein said contacting comprises delivery of said ethanol at different concentrations in the range of approximately 5-20 mM.

16. The method of claim 14, wherein said contacting comprises delivery of said ethanol at different frequencies.

17. The method of claim 14, wherein said contacting comprises delivery of said ethanol at different durations.

18. The method of claim 14, further comprising contacting said hepatic cells with a solution lacking alcohol before step c) wherein said at least one alcoholic liver disease stage is selected from fatty liver tissue, alcoholic steatohepatitis, liver fibrosis, liver cirrhosis and hepatic carcinoma.

19. The method of claim 14, wherein said indicator of a disease phenotype at least one alcoholic liver disease biomarker is selected from the group consisting of lipid droplets, cytochrome P450 induction, hepatocyte apoptosis, liver sinusoidal endothelial cell apoptosis, free radical generation, and mitochondrial damage.

20. The method of claim 14, wherein said microfluidic device further comprises an inlet channel and an outlet channel in fluidic communication with said one or more microfluidic channels.

21. The method of claim 20, wherein said inlet channel delivers said physiological buffer solution to said one or more channels.

22. The method of claim 20, wherein said outlet channel removes said physiological buffer solution from said one or more channels.

23. The method of claim 14, further comprising d) screening a drug for liver cell injury therapy flowing said physiological buffer solution into said one or more channels with said inlet channel.

24. The method of claim 14, further comprising flowing said physiological buffer wherein said solution further comprises a pro-inflammatory compound out of said one or more channels with said outlet channel.

25-34. (canceled)

35. A method, comprising:
a) providing;
i) a microfluidic device comprising a membrane comprising first and second surfaces, and first and second microfluidic channels;
ii) a collagen gel on said first surface;
iii) a plurality of hepatic cells on or in said collagen gel, said hepatic cells covered by a collagen overlay in said first microfluidic channel; and
b) flowing media through said first channel under conditions wherein bile canaliculi networks form.

36. The method of claim 35, wherein said collagen gel comprises stellate cells.

37. The method of claim 35, further comprising endothelial cells in said second channel.

38. The method of claim 35, further comprising Kupffer cells in said second channel.

39. The method of claim 35, wherein said collagen gel on said first surface comprises a 3D underlay.

40. The method of claim 35, wherein said collagen overlay comprises a 3D overlay.

41. The method of claim 35, wherein said collagen gel on said first surface comprises Collagen I.

42. The method of claim 35, wherein the concentration of Collagen I is 0.5 mg/ml.

43. A method, comprising:
a) providing;
i) a microfluidic device comprising a membrane comprising first and second surfaces, and first and second microfluidic channels;
ii) a collagen gel on said first surface;
iii) a plurality of hepatic cells on or in said collagen gel, said hepatic cells covered by a collagen overlay in said first microfluidic channel;
b) flowing media through said first channel under conditions wherein bile canaliculi networks form and said hepatic cells express a bile canaliculi biomarker; and
c) introducing a drug under conditions such that expression of said bile canaliculi biomarker is reduced.

44. The method of claim 43, wherein said collagen gel comprises stellate cells.

45. The method of claim 43, wherein said bile canaliculi biomarker is Multidrug resistance-associated protein 2.

46. The method of claim 43, wherein said hepatic cells are hepatocytes.

47. The method of claim 43, further comprising endothelial cells in said second channel.

48. The method of claim 43, further comprising Kupffer cells in said second channel.

49. The method of claim 43, wherein said collagen gel on said first surface comprises a 3D underlay.

50. The method of claim 43, wherein said collagen overlay comprises a 3D overlay.

51. The method of claim 43, wherein said collagen gel on said first surface comprises Collagen I.

52. The method of claim 43, wherein the concentration of Collagen I is 0.5 mg/ml.

53. A method for evaluating dysregulation of biliary function, comprising:
a) providing;
i) a microfluidic device comprising a solid substrate, said solid substrate comprising a membrane, one or more microfluidic channels and hepatic cells;
ii) a solution comprising a physiologically relevant concentration of ethanol;
b) culturing said hepatic cells such that bile canaliculi structures form;
c) exposing said hepatic cells to ethanol by contacting said hepatic cells with said solution; and
c) quantifying said bile canaliculi structures before and after said ethanol exposure.

54. The method of claim 53, wherein said contacting comprises delivery of said ethanol at different concentrations in the range of approximately 5-20 mM.

55. The method of claim 53, wherein said contacting comprises delivery of said ethanol at different frequencies.

56. The method of claim 53, wherein said contacting comprises delivery of said ethanol at different durations of time.

57. The method of claim 53, wherein said quantifying comprising exposing said hepatic cells to Calcein-AM.

58. The method of claim 53, wherein said microfluidic device further comprises an inlet channel and an outlet channel in fluidic communication with said one or more microfluidic channels.

59. The method of claim 58, wherein said inlet channel delivers said solution to said one or more channels.

60. The method of claim 58, wherein said outlet channel removes said solution from said one or more channels.

61. The method of claim 58, further comprising flowing said solution into said one or more channels with said inlet channel.

62. The method of claim 58, further comprising flowing said solution out of said one or more channels with said outlet channel.

63. A method, comprising:
a) providing;
i) a microfluidic device comprising a solid substrate, said solid substrate comprising a membrane, one or more microfluidic channels and hepatic cells;
ii) a solution comprising LPS and a physiologically relevant concentration of ethanol;
b) exposing said hepatic cells to ethanol and a proinflammatory cytokine by contacting said hepatic cells with said solution.

64. The method of claim 63, further comprising c) measuring an indicator of a disease phenotype of said hepatic cells.

65. The method of claim 63, further comprising c) measuring oxidative stress of said hepatic cells.

66. The method of claim 63, further comprising c) measuring lipid accumulation.

67. The method of claim 63, wherein said contacting comprises delivery of said ethanol at a concentration in the range of approximately 5-20 mM.

68. The method of claim 65, further comprising comparing the level of oxidative stress of said hepatic cells with hepatic cells exposed to ethanol alone.

69. The method of claim 68, further comprising detecting an increase in oxidative stress with the combination of ethanol and said proinflammatory cytokine.

70. The method of claim 65, wherein oxidative stress is measured using a dye.

71. The method of claim 63, further comprising removing said ethanol and said proinflammatory cytokine and culturing said hepatic cells for a number of days in the absence of ethanol and said proinflammatory cytokine, so as to create recovered hepatic cells.

72. The method of claim 71, further comprising, after said number of days, measuring oxidative stress of said recovered hepatic cells.

73. The method of claim 72, further comprising comparing the level of oxidative stress of said recovered hepatic cells with recovered hepatic cells exposed to ethanol alone.

74. The method of claim 72, further comprising detecting an increase in oxidative stress in said recovered hepatic cells with the combination of ethanol and said proinflammatory cytokine.

75. The method of claim 63, wherein said proinflammatory cytokine comprises LPS.

76. A device, comprising i) a microfluidic device comprising a membrane comprising first and second surfaces, and first and second microfluidic channels, ii) a collagen gel on said first surface; iii) a plurality of hepatocytes on or in said collagen gel, said hepatocytes covered by a collagen overlay in said first microfluidic channel.

77. The device of claim 76, wherein said collagen gel comprises stellate cells.

78. The device of claim 76, further comprising endothelial cells in said second channel.

79. The device of claim 76, further comprising Kupffer cells in said second channel.

80. The device of claim 76, wherein said collagen gel on said first surface comprises a 3D underlay.

81. The device of claim 76, wherein said collagen overlay comprises a 3D overlay.

82. The device of claim 76, wherein said collagen gel on said first surface comprises Collagen I.

83. The device of claim 76, wherein the concentration of Collagen I is 0.5 mg/ml.
